(19) United States
(12) Patent Application Publication
(10) Pub. No.: US 2009/0238761 A1
(43) Pub. Date: Sep. 24, 2009

(54) Novel Aryl Piperazine Derivatives With Medical Utility

(75) Inventors: Giuseppe Campiani, Chianciano Terme (Siena) (IT); Stefania Butini, Siena (IT); Caterina Fattorusso, Napoli (IT); Francesco Trotta, Cerreto Sannita (Benevento) (IT); Silvia Franceschini, Castel Del Piano (IT); Meri De Angelis, Cossingnano (IT); Karin Sandager Nielsen, Fredensborg (DK)

Correspondence Address: BIRCH STEWART KOLASCH & BIRCH, PO BOX 747, FALLS CHURCH, VA, 22040-0747, US

(73) Assignee: Universita Degli Studi Di Siena, Siena (IT)

(21) Appl. No.: 11/794,687

(22) PCT Filed: Jan. 2, 2006
(86) PCT No.: PCT/EP2006/050001

§ 371 Date: Sep. 4, 2007

Related U.S. Application Data
(60) Provisional application No. 60/641,006, filed on Jan. 4, 2005.

(30) Foreign Application Priority Data
Jan. 3, 2005 (DK) PA 2005 00004

(51) Int. Cl. A61K 49/00; C07D 405/02; A61K 31/496; C07D 241/36; A61K 31/4985; C07D 401/02; C07D 471/02; A61K 31/437; C07D 241/04; A61P 25/00

(52) U.S. Cl. 424/9.1; 544/376; 514/254.11; 544/344; 514/250; 544/363; 514/253.05; 546/86; 514/292; 544/357; 514/252.11

(57) ABSTRACT

This invention provides novel aryl piperazine derivatives having medical utility, in particular as modulators of dopamine and serotonin receptors, preferably the D₃, D₂-like and 5-HT₂ receptor subtypes, and in particular useful for the treatment of neuropsychiatric disorders incl. schizophrenia.

### TECHNICAL FIELD

This invention provides novel aryl piperazine derivatives having medical utility, in particular as modulators of dopamine and serotonin receptors, preferably the D₃, D₂-like and 5-HT₂ receptor subtypes, and in particular useful for the treatment of neuropsychiatric disorders incl. schizophrenia.

### BACKGROUND ART

Dopamine is involved in several important functions, excitatory and inhibitory, via dopaminergic receptors in the central and peripherical nervous system. Dopamine receptors were originally classified into two main groups: D₁ and D₂. The five currently cloned dopamine receptors fall into these classes. Thus, the D₁-like receptors include D₁ and D₅, while the D₂-like receptors include D₂, D₃ and D₄.

The dopamine receptors, and in particular the D₂-like receptors, are recognised as potential therapeutic targets for various neurological and psychiatric disorders, in particular psychotic disorders, incl. schizophrenia. Other therapeutic indications associated with the dopamine receptors include depression, Parkinson's disease, Huntington's disease, movement disorders such as dystonia, anxiety, restlessness, obsessive-compulsive disorders, mania, geriatric disorders, dementia, sexual dysfunction, musculo-skeletal pain symptoms, e.g. pain associated with fibromyalgia, substance abuse (cocaine abuse and addiction), withdrawal symptoms in drug addicts, and sleep disorders.

Finally receptor selective ligands find use as diagnostic tools in diagnostic methods, and in particular for in vivo receptor imaging (neuroimaging).

Dopamine receptor selective ligands have been described in e.g. WO 2004024878, and by i.a. Leopoldo et al. (J. Med. Chem. 2002 45 5727-5733), Campiani et al. (J. Med. Chem. 2003 46 3822-3839) and Hackling et al. (J. Med. Chem. 2003 46 3883-3899).

### SUMMARY OF THE INVENTION

According to the present invention it has now been found that certain aryl piperazine derivatives show superior activity as modulators of dopamine and serotonin receptors, preferably the D₃, D₂-like and 5-HT₂ receptor subtypes, and thus are particular useful as antipsychotic agents.

Therefore, in its first aspect, the invention provides novel aryl piperazine derivatives represented by Formula I

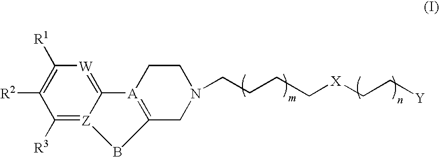

an enantiomer thereof or a mixture of its enantiomers, or a pharmaceutically acceptable salt thereof, or an N-oxide thereof, wherein,

R¹, R² and R³, independently of one another, represent hydrogen, alkyl, cycloalkyl, cycloalkyl-alkyl, alkenyl, hydroxy, alkoxy, cycloalkoxy, halo, haloalkyl, haloalkoxy, amino, nitro cyano and/or carboxy;

[symbol] represents an optional double bond; if [symbol] represents a single bond, then A represents CH or N; if [symbol] represents a double bond, then A represents C;

—B— may be absent or present: —B— is absent; and Z represents CH or N; or —B— is present and represents a methylene bridge (—CH₂—), an ethylene bridge (═CH—), or a bridge —NH—, attached as indicated in the figure; and Z represents C (carbon);

W represents CH, N or CR⁴, wherein R⁴ represents represent hydrogen, alkyl, cycloalkyl, cycloalkyl-alkyl, alkenyl, hydroxy, alkoxy, cycloalkoxy, halo, haloalkyl, haloalkoxy, amino, nitro or cyano;

m and n, independently of each other, is 0, 1 or 2; and

X may be absent or present:

X is present and represents O, S, NR′, CO, SO₂, CH₂, CH₂—O, O—CH₂, CH₂—S, S—CH₂, CH₂—NR′, CH₂—CO, CH₂—SO₂, NR′—CO, CO—NR′, NR′—SO₂, SO₂—NR′, CH₂—CH₂, O—CO, CO—O, O—CH═CH, S—CH═CH, NR′—CH═CH, CO—CH═CH, SO₂—CH═CH, CH₂—O—CH═CH, CH₂—S—CH═CH, CH₂—NR′—CH═CH, CH₂—CO—CH═CH, CONHCH₂CH₂ or CH₂—SO₂—CH═CH, wherein R′ represents hydrogen or alkyl; and

Y represents phenyl or an aromatic monocyclic or polycyclic heterocyclic group, which phenyl or heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano; or

Y represents a hydrogenated heterocyclic group, which hydrogenated heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano; or

Y represents a group of formula III

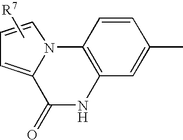

wherein

R⁷ represents hydrogen, alkyl, alkoxy, halo or haloalkyl; or

X is absent; and

Y represents a diazacyclic group of Formula II,

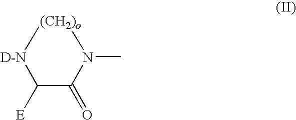

wherein,

o is 1, 2 or 3;

D represents alkyl, cycloalkyl, cycloalkyl-alkyl, hydroxy, alkoxy, cycloalkoxy, halo, haloalkyl, haloalkoxy, amino, nitro and cyano; and

E represents alkyl, cycloalkyl, cycloalkyl-alkyl, hydroxy, alkoxy, cycloalkoxy, halo, haloalkyl, haloalkoxy, amino, nitro and cyano; or

D and E together with the diazacyclic group form a fused ring system, which fused ring system may optionally be substituted one or more times with substituents selected from the group consisting of alkyl, cycloalkyl, cycloalkyl-alkyl, hydroxy, alkoxy, cycloalkoxy, halo, haloalkyl, haloalkoxy, amino, nitro and cyano;

or Y represents a group of formula IV

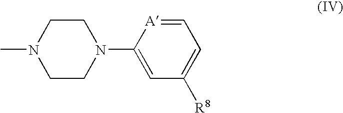

wherein A′ represents CH or N; and

R⁸ represents hydrogen, alkyl, alkoxy, halo or haloalkyl.

In another aspect the invention relates to the use of the aryl piperazine derivative of the invention, or a pharmaceutically acceptable salt thereof, or a prodrug thereof for the manufacture of a pharmaceutical composition.

Viewed from yet another aspect the invention relates to the use of the aryl piperazine derivative of the invention, or a pharmaceutically acceptable salt thereof, or a prodrug thereof, for the manufacture of a pharmaceutical composition for the treatment, prevention or alleviation of a disease or a disorder or a condition of a mammal, including a human, which disease, disorder or condition is responsive to modulation of the dopamine and serotonin receptors.

In a final aspect the invention provides a method of diagnosis, treatment, prevention or alleviation of a disease or a disorder or a condition of a living animal body, including a human, which disorder, disease or condition is responsive to modulation of the dopamine and serotonin receptors, in particular the D₃, D₂-like and 5-HT₂ receptor subtypes, preferably the dopamine D₃ receptor subtype and/or the D₃/5-HT_{1A} or D₃/5-HT_{2A} receptor subtypes, which method comprises the step of administering to such a living animal body in need thereof, a therapeutically effective amount of the aryl piperazine derivative of the invention, or a pharmaceutically acceptable salt thereof, or a prodrug thereof.

Other objects of the invention will be apparent to the person skilled in the art from the following detailed description and examples.

### DETAILED DISCLOSURE OF THE INVENTION

According to the present invention it has now been found that a particular group of aryl piperazine derivatives show a superior biological profile as modulators of dopamine and serotonin receptors.

Therefore, in its first aspect, the invention provides novel aryl piperazine derivatives represented by Formula I

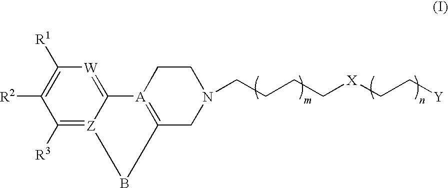

an enantiomer thereof or a mixture of its enantiomers, or a pharmaceutically acceptable salt thereof, or an N-oxide thereof, wherein,

R¹, R² and R³, independently of one another, represent hydrogen, alkyl, cycloalkyl, cycloalkyl-alkyl, alkenyl, hydroxy, alkoxy, cycloalkoxy, halo, haloalkyl, haloalkoxy, amino, nitro cyano and/or carboxy;

[symbol] represents an optional double bond; if [symbol] represents a single bond, then A represents CH or N; if [symbol] represents a double bond, then A represents C;

—B— may be absent or present: —B— is absent; and Z represents CH or N; or —B— is present and represents a methylene bridge (—CH₂—), an ethylene bridge (═CH—), or a bridge —NH—, attached as indicated in the figure; and Z represents C (carbon);

W represents CH, N or CR⁴, wherein R⁴ represents represent hydrogen, alkyl, cycloalkyl, cycloalkyl-alkyl, alkenyl, hydroxy, alkoxy, cycloalkoxy, halo, haloalkyl, haloalkoxy, amino, nitro or cyano;

m and n, independently of each other, is 0, 1 or 2; and

X may be absent or present:

X is present and represents O, S, N′, CO, SO₂, CH₂, CH₂—O, O—CH₂, CH₂—S, S—CH₂, CH₂—NR′, CH₂—CO, CH₂—SO₂, NR′—CO, CO—NR′, NR′—SO₂, SO₂—NR′, CH₂—CH₂, O—CO, CO—O, O—CH═CH, S—CH═CH, NR′—CH═CH, CO—CH═CH, SO₂—CH═CH, CH₂—O—CH═CH, CH₂—S—CH═CH, CH₂—NR′—CH═CH, CH₂—CO—CH═CH, CONHCH₂CH₂ or CH₂—SO₂—CH═CH, wherein R′ represents hydrogen or alkyl; and

Y represents phenyl or an aromatic monocyclic or polycyclic heterocyclic group, which phenyl or heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano; or Y represents a hydrogenated heterocyclic group, which hydrogenated heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano; or

Y represents a group of formula III

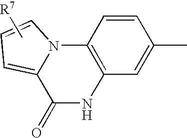

wherein

R⁷ represents hydrogen, alkyl, alkoxy, halo or haloalkyl; or

X is absent; and

Y represents a diazacyclic group of Formula II,

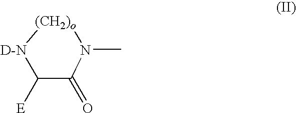

wherein,

o is 1, 2 or 3;

D represents alkyl, cycloalkyl, cycloalkyl-alkyl, hydroxy, alkoxy, cycloalkoxy, halo, haloalkyl, haloalkoxy, amino, nitro and cyano; and

E represents alkyl, cycloalkyl, cycloalkyl-alkyl, hydroxy, alkoxy, cycloalkoxy, halo, haloalkyl, haloalkoxy, amino, nitro and cyano; or

D and E together with the diazacyclic group form a fused ring system, which fused ring system may optionally be substituted one or more times with substituents selected from the group consisting of alkyl, cycloalkyl, cycloalkyl-alkyl, hydroxy, alkoxy, cycloalkoxy, halo, haloalkyl, haloalkoxy, amino, nitro and cyano;

or Y represents a group of formula IV

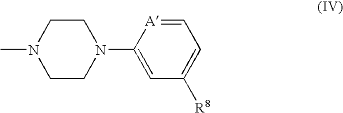

wherein A′ represents CH or N; and

R⁸ represents hydrogen, alkyl, alkoxy, halo or haloalkyl.

In a more preferred embodiment the aryl piperazine derivative of the invention is a compound of Formula I, wherein

R¹, R² and R³, independently of one another, represent hydrogen, alkyl, cycloalkyl, cycloalkyl-alkyl, alkenyl, hydroxy, alkoxy, cycloalkoxy, halo, haloalkyl, haloalkoxy, amino, nitro and/or cyano, carboxy;

[symbol] represents an optional double bond; if [symbol] represents a single bond, then A represents CH or N; if [symbol] represents a double bond, then A represents C (carbon);

—B— may be absent or present: —B— is absent; and Z represents CH or N; or —B— is present and represents a methylene bridge (—CH₂—), an ethylene bridge (═CH—), or a bridge —NH—, attached as indicated in the figure; and

Z represents C (carbon);

W represents CH, N or CR⁴, wherein R⁴ represents represent hydrogen, alkyl, cycloalkyl, cycloalkyl-alkyl, alkenyl, hydroxy, alkoxy, cycloalkoxy, halo, haloalkyl, haloalkoxy, amino, nitro or cyano;

m and n, independently of each other, is 0, 1 or 2; and

X may be absent or present:

X is present and represents O, S, NR′, CO, SO₂, CH₂, CH₂—O, O—CH₂, CH₂—S, S—CH₂, CH₂—NR′, CH₂—CO, CH₂—SO₂, NR′—CO, CO—NR′, NR′—SO₂, SO₂—NR′, CH₂—CH₂, O—CO, CO—O, O—CH═CH, S—CH═CH, NR′—CH═CH, CO—CH═CH, SO₂—CH═CH, CH₂—O—CH═CH, CH₂—S—CH═CH, CH₂—NR′—CH═CH, CH₂—CO—CH═CH, CONHCH₂CH₂ or CH₂—SO₂—CH═CH, wherein R′ represents hydrogen or alkyl; and

Y represents phenyl or an aromatic monocyclic or polycyclic heterocyclic group, which phenyl or heterocyclic group may optionally be substituted one or more times with substituents selected from the group consisting of alkyl, cycloalkyl,

[symbol]

In a most preferred embodiment W represents CH or N.

In a fourth preferred embodiment the aryl piperazine derivative of the invention is a compound of Formula I, wherein —B— is absent, and Z represents CH or N.

In a more preferred embodiment —B— is present and represents a methylene bridge (—CH₂—), an ethylene bridge (═CH—), or a bridge —NH—, attached as indicated in the figure; and Z represents C (carbon).

In an even more preferred embodiment —B— is present and represents a methylene bridge (—CH₂—), an ethylene bridge (═CH—), or a bridge —NH—, attached as indicated in the figure; and Z represents C (carbon); and W represents CR⁴, wherein R⁴ represents represent hydrogen, alkyl, in particular methyl, alkoxy, in particular methoxy, halo, in particular chloro, haloalkyl, haloalkoxy, amino, nitro or cyano.

In a fourth preferred embodiment the aryl piperazine derivative of the invention is a compound of Formula I, wherein W represents CR⁴, wherein R⁴ represents represent hydrogen, alkyl, in particular methyl, alkoxy, in particular methoxy or halo, in particular chloro.

In a more preferred embodiment W represents CR⁴, wherein R⁴ represents represent hydrogen, methyl, ethyl, methoxy, fluoro or chloro.

In an even more preferred embodiment W represents CR⁴, wherein R⁴ represents represent hydrogen, alkyl or alkoxy[methoxy].

In a fifth preferred embodiment the aryl piperazine derivative of the invention is a compound of Formula I, wherein m and n, independently of each other, is 0, 1 or 2.

In a more preferred embodiment m is 1 or 2; and n is 0 or 2.

In an even more preferred embodiment m is 1; and n is 0.

In another preferred embodiment m is 1; and n is 1.

In a third preferred embodiment m is 1; and n is 2.

In a still more preferred embodiment m is 2; and n is 0.

In a sixth preferred embodiment the aryl piperazine derivative of the invention is a compound of Formula I, wherein R¹, R² and R³, independently of one another, represent hydrogen, alkyl, in particular methyl, ethyl or propyl, cycloalkyl, cycloalkyl-alkyl, alkenyl, hydroxy, alkoxy, in particular methoxy or ethoxy, cycloalkoxy, halo, in particular fluoro, chloro or bromo, haloalkyl, in particular trifluoromethyl, haloalkoxy, amino, nitro cyano and/or carboxy;

In a more preferred embodiment R¹, R² and R³ represent hydrogen.

In an even more preferred embodiment R¹ represents alkyl, in particular methyl, ethyl or propyl, cycloalkyl, cycloalkyl-alkyl, hydroxy, alkoxy, in particular methoxy or ethoxy, cycloalkoxy, halo, in particular fluoro, chloro or bromo, haloalkyl, in particular trifluoromethyl, haloalkoxy, amino, nitro, cyano or carboxy; and R² and R³ represent hydrogen.

In a still more preferred embodiment R¹ represents alkyl, in particular methyl, ethyl or propyl, cycloalkyl, cycloalkyl-alkyl, hydroxy, alkoxy, in particular methoxy or ethoxy, cycloalkoxy, halo, in particular fluoro, chloro or bromo, haloalkyl, in particular trifluoromethyl, haloalkoxy, amino, nitro or cyano; and R² and R³ represent hydrogen.

In a yet more preferred embodiment R¹ represents alkyl, in particular methyl, ethyl or propyl, cycloalkyl, cycloalkyl-alkyl, hydroxy, alkoxy, in particular methoxy or ethoxy, cycloalkoxy, halo, in particular fluoro, chloro or bromo, haloalkyl, in particular trifluoromethyl, haloalkoxy, amino, nitro or cyano.

In a further preferred embodiment R¹ represents alkyl, in particular methyl, ethyl or propyl, alkoxy, in particular methoxy or ethoxy, halo, in particular fluoro, chloro or bromo, haloalkyl, in particular trifluoromethyl, haloalkoxy, amino, nitro or cyano.

In a still further preferred embodiment R¹ represents methyl, ethyl, methoxy, chloro, trifluoromethyl, trifluoromethoxy or cyano.

In a still further preferred embodiment R¹ represents methyl, ethyl, methoxy, chloro, trifluoromethyl, trifluoromethoxy, cyano or carboxy.

In another preferred embodiment R² represents alkyl, cycloalkyl, cycloalkyl-alkyl, hydroxy, alkoxy, cycloalkoxy, halo, in particular chloro, haloalkyl, haloalkoxy, amino, nitro or cyano; and R¹ and R³ represent hydrogen.

In a more preferred embodiment R² represents alkyl, cycloalkyl, alkoxy, halo, trifluoromethyl, trifluoromethoxy, amino, nitro or cyano.

In an even more preferred embodiment R² represents methyl, ethyl, methoxy, chloro, trifluoromethyl, trifluoromethoxy or cyano.

In a seventh preferred embodiment the aryl piperazine derivative of the invention is a compound of Formula I, wherein X is present and represents O, S, NR′, CO, SO₂, CH₂, CH₂—O, O—CH₂, CH₂—S, S—CH₂, CH₂—NR′, CH₂—CO, CH₂—SO₂, NR′—CO, CO—NR′, CH₂—CH₂, O—CO, CO—O, O—CH═CH, S—CH═CH, NR′—CH═CH, CO—CH═CH, SO₂—CH═CH, CH₂—O—CH═CH, CH₂—S—CH═CH, CH₂—NR′—CH═CH, CH₂—CO—CH═CH, CONHCH₂CH₂ or CH₂—SO₂—CH═CH, wherein R′ represents hydrogen or alkyl.

In a more preferred embodiment X represents O, CH₂—O, O—CH₂, CH₂—S, S—CH₂, CH₂—NR′, CH₂—CO, CH₂—SO₂, NR′—CO, CO—NR′, NR′—SO₂, SO₂—NR′, O—CO, or CH₂—O—CH═CH; wherein R′ represents hydrogen or alkyl.

In an even more preferred embodiment X represents O, CH₂—O, O—CH₂, CH₂—S, S—CH₂, CH₂—NR′, CH₂—CO, CH₂—SO₂, NR′ CO, CO—NR′, O—CO, or CH₂—O—CH═CH; wherein R′ represents hydrogen or alkyl.

[symbol]

In a most preferred embodiment Y represents pyridyl, optionally substituted with methyl, ethyl, methoxy, chloro or trifluoromethyl.

In a tenth preferred embodiment the aryl piperazine derivative of the invention is a compound of Formula I, wherein Y represents an aromatic bicyclic heterocyclic group selected from indolyl, isoindolyl, benzo[b]furanyl, benzo[b]thienyl, benzimidazolyl, benzthiazolyl, quinolinyl and isoquinolinyl, which aromatic bicyclic heterocyclic group may optionally be substituted one or two times with substituents selected from the group consisting of alkyl, in particular methyl, cycloalkyl, cycloalkyl-alkyl, hydroxy, alkoxy, cycloalkoxy, halo, in particular chloro, haloalkyl, haloalkoxy, amino, nitro and cyano.

In a more preferred embodiment Y represents indolyl, in particular indol-2-yl or indol-3-yl; benzo[b]furanyl, in particular benzo[b]furan-2-yl or benzo[b]furan-3-yl; benzo[b]thienyl, in particular benzo[b]thien-2-yl or benzo[b]thien-3-yl; quinolinyl in particular quinolin-2-yl, quinolin-3-yl or quinolin-4-yl; or isoquinolinyl, in particular isoquinolin-1-yl, isoquinolin-3-yl, or isoquinolin-4-yl; which aromatic bicyclic heterocyclic group may optionally be substituted one or two times with substituents selected from alkyl, in particular methyl, cycloalkyl, cycloalkyl-alkyl, hydroxy, alkoxy, cycloalkoxy, halo, in particular chloro, haloalkyl, haloalkoxy, amino, nitro and cyano.

In an even more preferred embodiment Y represents indolyl, in particular indol-2-yl or indol-3-yl; benzo[b]furanyl, in particular benzo[b]furan-2-yl or benzo[b]furan-3-yl; quinolinyl, in particular quinolin-2-yl, quinolin-3-yl or quinolin-4-yl; or isoquinolinyl, in particular isoquinolin-1-yl, isoquinolin-3-yl, or isoquinolin-4-yl; which benzo[b]furanyl or isoquinolinyl may optionally be substituted one or two times with substituents selected from alkyl, in particular methyl, hydroxy, alkoxy, chloro, trifluoromethyl, trifluoromethoxy, amino, nitro and cyano.

In a still more preferred embodiment Y represents indol-2-yl, benzo[b]furan-2-yl or isoquinolin-3-yl; which benzo[b]furanyl or isoquinolinyl may optionally be substituted one or two times with substituents selected from alkyl, in particular methyl, hydroxy, alkoxy, chloro, trifluoromethyl, trifluoromethoxy, amino, nitro and cyano.

In a yet more preferred embodiment Y represents indol-2-yl, benzo[b]furan-2-yl or isoquinolin-3-yl; which benzo[b]furanyl or isoquinolinyl may optionally be substituted with alkyl, in particular methyl, halo, in particular chloro, or trifluoromethyl.

In a further preferred embodiment Y represents indol-2-yl, benzo[b]furan-2-yl or isoquinolin-3-yl; which benzo[b]furanyl or isoquinolinyl may optionally be substituted with methyl, ethyl, fluoro, chloro or trifluoromethyl.

In a most preferred embodiment Y represents indolyl, benzo[b]furanyl, or isoquinolinyl.

In an eleventh preferred embodiment the aryl piperazine derivative of the invention is a compound of Formula I, wherein Y represents a hydrogenated heterocyclic group, in particular tetrahydroquinolinyl, which hydrogenated heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano.

In a more preferred embodiment Y represents tetrahydroquinolinyl or tetrahydroisoquinolinyl, which heterocyclic group may optionally be substituted one or two times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano.

In a most preferred embodiment Y represents tetrahydroquinolinyl or tetrahydroisoquinolinyl.

In another preferred embodiment X represents O, CH₂—O, NH—CO, CO—NH, NR′—SO₂ or CO—O; and Y represents phenyl, methyl-phenyl, pyridyl, indolyl, methyl-indolyl, benzo[b]furanyl, tetrahydroquinolinyl, isoquinolinyl, or tetrahydroisoquinolinyl.

In a more preferred embodiment X represents CH₂—O, NH—CO, CO—NH or CO—O; and Y represents indolyl, benzo[b]furanyl, tetrahydroquinolinyl, isoquinolinyl, or tetrahydroisoquinolinyl.

In an even more preferred embodiment X represents O, CH₂—O, NH—CO, CO—NH, NR′—SO₂ or CO—O; Y represents phenyl, methyl-phenyl, pyridyl, methyl-pyridyl, indolyl, methyl-indolyl, benzo[b]furanyl, tetrahydroquinolinyl, isoquinolinyl, or tetrahydroisoquinolinyl; R¹ represents alkyl, cycloalkyl, cycloalkyl-alkyl, hydroxy, alkoxy, cycloalkoxy, halo, haloalkyl, haloalkoxy, amino, nitro or cyano; and R² and R³ represent hydrogen.

In a yet more preferred embodiment X represents CH₂—O, NH—CO, CO—NH or CO—O; Y represents indolyl, benzo[b]furanyl, tetrahydroquinolinyl, isoquinolinyl, or tetrahydroisoquinolinyl; R¹ represents alkyl, cycloalkyl, cycloalkyl-alkyl, hydroxy, alkoxy, cycloalkoxy, halo, haloalkyl, haloalkoxy, amino, nitro or cyano; and R² and R³ represent hydrogen.

In a most preferred embodiment the aryl piperazine derivative of the invention is
N-[4-[4-(3-Trifluoromethylphenyl)piperazin-1-yl]butyl]indole-2-carboxamide;
N-[2-(1H-Indol-3-yl)ethyl]-3-(4-m-tolylpiperazin-1-yl)propanamide;
N-[2-(1H-Indol-3-yl)ethyl]-3-[4-(3-methoxyphenyl)piperazin-1-yl]propanamide;
Benzo[b]furan-2-carboxylic acid {4-[4-(3-methoxy-phenyl)-piperazin-1-yl]-butyl}-amide;
N-[4-[4-(3-Cyanophenyl)piperazin-1-yl]butyl]benzo[b]furan-2-carboxamide;
Benzo[b]furan-2-carboxylic acid {4-[4-(3-chloro-phenyl)-piperazin-1-yl]-butyl}-amide;
Benzo[b]furan-2-carboxylic acid {4-[4-(3-carboxy-phenyl)-piperazin-1-yl]-butyl}-amide;
N-[4-[4-(m-Tolyl)piperazin-1-yl]butyl]benzo[b]furan-2-carboxamide;
Isoquinoline-3-carboxylic acid {4-[4-(3-cyano-phenyl)-piperazin-1-yl]-butyl}-amide;
N-[4-[4-(3-Chlorophenyl)piperazin-1-yl]butyl]isoquinoline-3-carboxamide;
N-[4-[4-(m-Tolyl)piperazin-1-yl]butyl]isoquinoline-3-carboxamide;
N-[4-[4-(3-Methoxyphenyl)piperazin-1-yl]butyl]isoquinoline-3-carboxamide;
3-[5-[4-(3-Chlorophenyl)piperazin-1-yl]pentyloxy]isoquinoline;
3-{5-[4-(3-Methoxy-phenyl)-piperazin-1-yl]-pentyloxy}-isoquinoline;
3-[5-(4-m-Tolylpiperazin-1-yl)pentyloxy]isoquinoline;
3-{5-[4-(3-Cyano-phenyl)-piperazin-1-yl]-pentyloxy}-isoquinoline;
N-[4-(1,2,3,4-Tetrahydro-5-methoxy-β-carbolin-2-yl)butyl]isoquinoline-3-carboxamide;
N-[4-(3,4-Dihydro-6-methoxypyrazino[1,2-a]indol-2(1H)-yl)butyl]isoquinoline-3-carboxamide;
N-[4-[4-(Pyridin-2-yl)piperazin-1-yl]butyl]isoquinoline-3-carboxamide;
1,2,3,4-Tetrahydro-quinoline-2-carboxylic acid [4-(4-phenyl-piperazin-1-yl)-butyl]-amide;
(S)-(−)-N-[4-[4-(m-Tolyl)piperazin-1-yl]butyl]-1,2,3,4-tetrahydroisoquinoline-2-carboxamide;
(R)-(+)-N-[4-[4-(m-Tolyl)piperazin-1-yl]butyl]-1,2,3,4-tetrahydroisoquinoline-2-carboxamide;
1H-Indole-2-carboxylic acid {4-[4-(2,4-dichloro-phenyl)-piperazin-1-yl]-butyl}-amide;
5-Chloro-1H-indole-2-carboxylic acid {4-[4-(2,4-dichloro-phenyl)-piperazin-1-yl]-butyl}-amide;
Isoquinoline-3-carboxylic acid {4-[4-(2,3-dichloro-phenyl)-piperazin-1-yl]-butyl}-amide;
3-{4-[4-(2,3-Dichloro-phenyl)-piperazin-1-yl]-butoxy}-isoquinoline;
3-{5-[4-(2,3-Dichloro-phenyl)-piperazin-1-yl]-pentyloxy}-isoquinoline;
4-[4-(2,3-Dichlorophenyl)piperazin-1-yl]butyl 1H-indole-2-carboxylate;
N-(4-(4-(Phenylpiperazin-1-yl)butyl)benzo[b]furan-2-carboxamide;
Benzo[b]furan-2-carboxylic acid {4-[4-(2,3-dimethyl-phenyl)-piperazin-1-yl]-butyl}-amide;
N-(4-(4-(3-Methoxyphenyl)piperazin-1-yl)butyl)benzo[b]furan-2-carboxamide;
N-(4-(4-(6-Methylpyridin-2-yl)piperazin-1-yl)butyl)isoquinoline-3-carboxamide;
N-(4-(4-Phenylpiperazin-1-yl)butyl)isoquinoline-3-carboxamide;
N-(4-(4-(6-Methylpyridin-2-yl)piperazin-1-yl)butyl)benzo[b]furan-2-carboxamide;
N-(4-(4-Phenylpiperazin-1-yl)butyl)quinoline-2-carboxamide;
N-(4-(4-m-Tolylpiperazin-1-yl)butyl)quinoline-2-carboxamide;
N-(4-(4-(3-Methoxyphenyl)piperazin-1-yl)butyl)-1-methyl-1H-indole-2-carboxamide;
N-(4-(4-(3-Methoxyphenyl)piperazin-1-yl)butyl)-1H-indole-3-carboxamide;
(S)-1,2,3,4-Tetrahydro-N-(4-(4-phenylpiperazin-1-yl)butyl)quinoline-2-carboxamide;
N-(4-(4-m-Tolylpiperazin-1-yl)butyl)picolinamide;
N-(4-(4-(Quinolin-3-yl)piperazin-1-yl)butyl)isoquinoline-3-carboxamide;
N-(4-(4-(3-Methoxyphenyl)piperazin-1-yl)butyl)-6-methylpyridine-2-carboxamide;
N-(4-(4-(3-Methoxyphenyl)piperazin-1-yl)butyl)quinoline-3-carboxamide;
N-(4-(4-(Pyridin-2-yl)piperazin-1-yl)butyl)quinoline-3-carboxamide;
N-(4-(4-Phenylpiperazin-1-yl)butyl)picolinamide;
N-(4-(4-(3-Methoxyphenyl)piperazin-1-yl)butyl)picolinamide;
N-(4-(4-(3-Methoxyphenyl)piperazin-1-yl)butyl)benzamide;
N-(4-(4-m-Tolylpiperazin-1-yl)butyl)benzamide;
N-(4-(4-Phenylpiperazin-1-yl)butyl)nicotinamide;
N-(4-(4-(6-Methylpyridin-2-yl)piperazin-1-yl)butyl)benzamide;
N-(4-(4-(6-Methoxypyridin-2-yl)piperazin-1-yl)butyl)benzamide;
N-(4-(4-(6-Methoxypyridin-2-yl)piperazin-1-yl)butyl)picolinamide; or
N-(4-(4-(6-Methylpyridin-2-yl)piperazin-1-yl)butyl)picolinamide;

or a pharmaceutically acceptable salt thereof.

In a twelfth preferred embodiment the aryl piperazine derivative of the invention is a compound of Formula I, wherein Y represents a group of formula III

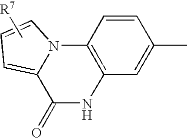

wherein R⁷ represents hydrogen, alkyl, alkoxy, halo or haloalkyl.

In a most preferred embodiment the aryl piperazine derivative of the invention is

7-[4-[4-(2,3-Dichloro-phenyl)-piperazin-1-yl]-butoxy]-pyrrolo[1,2-a]quinoxalin-4(5H)-one; 7-(5-(4-Phenylpiperazin-1-yl)pentyloxy)pyrrolo[1,2-a]quinoxalin-4(5H)-one; or

7-(4-(4-Phenylpiperazin-1-yl)butoxy)pyrrolo[1,2-a]quinoxalin-4(5H)-one;

or a pharmaceutically acceptable salt thereof.

In a thirteenth preferred embodiment the aryl piperazine derivative of the invention is a compound of Formula I, wherein X is absent; and Y represents a diazacyclic group of Formula II,

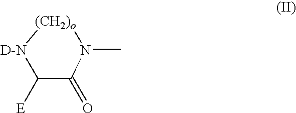

wherein, o is 1, 2 or 3;

D represents alkyl, cycloalkyl, cycloalkyl-alkyl, hydroxy, alkoxy, cycloalkoxy, halo, haloalkyl, haloalkoxy, amino, nitro and cyano; and E represents alkyl, cycloalkyl, cycloalkyl-alkyl, hydroxy, alkoxy, cycloalkoxy, halo, haloalkyl, haloalkoxy, amino, nitro and cyano; or D and E together with the diazacyclic group form a fused ring system, which fused ring system may optionally be substituted one or more times with substituents selected from the group consisting of alkyl, cycloalkyl, cycloalkyl-alkyl, hydroxy, alkoxy, cycloalkoxy, halo, haloalkyl, haloalkoxy, amino, nitro and cyano.

In a more preferred embodiment Y represents a bicyclic heterocyclic group (i.e. fused ring system) selected from the following group:

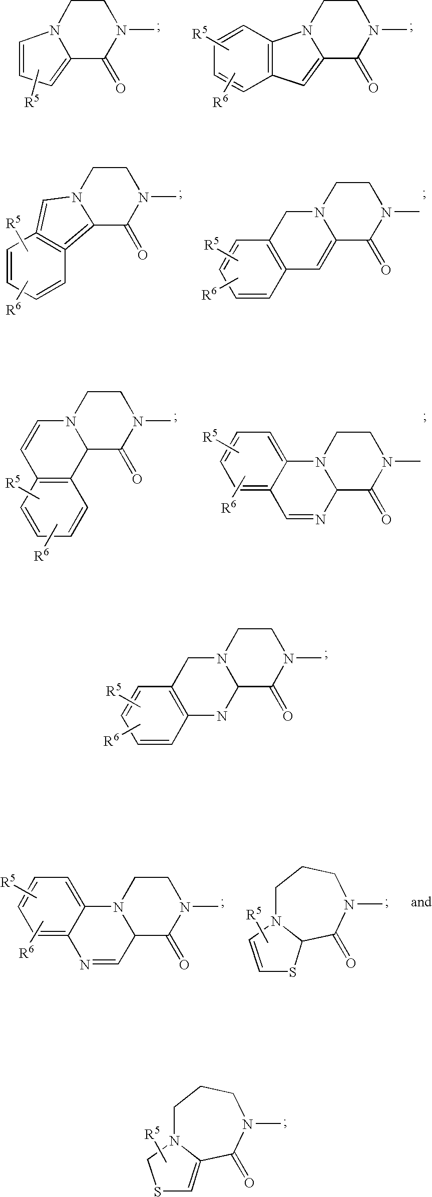

wherein R⁵ and R⁶, independently of each other, represent hydrogen, alkyl, cycloalkyl, cycloalkyl-alkyl, hydroxy, alkoxy, cycloalkoxy, halo, haloalkyl, haloalkoxy, amino, nitro and/or cyano.

In an even more preferred embodiment Y represents a bicyclic heterocyclic group selected from

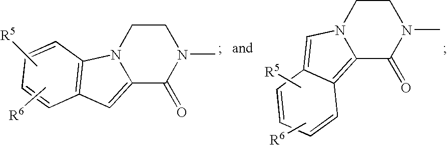

wherein R⁵ and R⁶, independently of each other, represent hydrogen, alkyl, cycloalkyl, cycloalkyl-alkyl, hydroxy, alkoxy, cycloalkoxy, halo, haloalkyl, haloalkoxy, amino, nitro and/or cyano.

In a yet more preferred embodiment Y represents

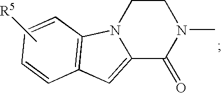

wherein R⁵ represents hydrogen, alkyl, halo, trifluoromethyl or trifluoromethoxy.

In a most preferred embodiment the aryl piperazine derivative of the invention is
2-{4-[4-(3-Cyano-phenyl)-piperazin-1-yl]-butyl}-3,4-dihydro-2H-pyrazino[1,2-a]indol-1-one;
2-[4-[4-(3-Chlorophenyl)piperazin-1-yl]butyl]-3,4-dihydropyrazino[1,2-a]indol-1(2H)-one;
2-{4-[4-(3-Methoxy-phenyl)-piperazin-1-yl]-butyl}-3,4-dihydro-2H-pyrazino[1,2-a]indol-1-one;
2-[4-(4-m-Tolyl)piperazin-1-yl]butyl]-3,4-dihydropyrazino[1,2-a]indol-1(2H)-one;
3,4-Dihydro-2-[4-(3,4-dihydro-6-methoxypyrazino[1,2-a]indol-2(1H)-yl)butyl]pyrazino[1,2-a]indol-1(2H)-one;
2-{4-[4-(2-Methoxy-phenyl)-piperazin-1-yl]-butyl}-3,4-dihydro-2H-pyrazino[1,2-a]indol-1-one; or
2-{4-[4-(2,3-Dichloro-phenyl)-piperazin-1-yl]-butyl}-3,4-dihydro-2H-pyrazino[1,2-a]indol-1-one;

or a pharmaceutically acceptable salt thereof.

In a fourteenth preferred embodiment the aryl piperazine derivative of the invention is a compound of Formula I, wherein X is absent; and Y represents a group of formula IV

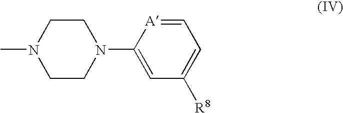

wherein A′ represents CH or N; and R⁸ represents hydrogen, alkyl, in particular methyl, alkoxy, in particular methoxy, halo, in particular chloro or haloalkyl.

In a most preferred embodiment the aryl piperazine derivative of the invention is
1,6-Bis(4-(3-chlorophenyl)piperazin-1-yl)hexane;
1,6-Bis(4-(3-methoxyphenyl)piperazin-1-yl)hexane;
1,6-Bis(4-phenylpiperazin-1-yl)hexane;
1-(3-Chlorophenyl)-4-(6-(4-(3-methoxyphenyl)piperazin-1-yl)hexyl)piperazine;
1-Phenyl-4-(6-(4-(pyridin-2-yl)piperazin-1-yl)hexyl)piperazine;
1-(6-Methylpyridin-2-yl)-4-(6-(4-m-tolylpiperazin-1-yl)hexyl)piperazine;
1-(6-Methylpyridin-2-yl)-4-(6-(4-phenylpiperazin-1-yl)hexyl)piperazine;
1-Phenyl-4-(6-(4-m-tolylpiperazin-1-yl)hexyl)piperazine;
4-(4-(6-(4-Phenylpiperazin-1-yl)hexyl)piperazin-1-yl)quinoline;
1,6-Bis(4-(pyridin-2-yl)piperazin-1-yl)hexane;
4-(4-(6-(4-m-Tolylpiperazin-1-yl)hexyl)piperazin-1-yl)quinoline;
1,6-Bis(4-m-tolylpiperazin-1-yl)hexane;
1-(Pyridin-2-yl)-4-(6-(4-m-tolylpiperazin-1-yl)hexyl)piperazine; or
1-(3-Methoxyphenyl)-4-(6-(4-m-tolylpiperazin-1-yl)hexyl)piperazine;

or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a haloalkyl group designates an alkyl group as defined herein, which alkyl group is substituted one or more times with halo. Preferred haloalkyl groups of the invention include trihalomethyl, preferably —CF₃.

In the context of this invention an alkoxy group designates an “alkyl-O—” group, wherein alkyl is as defined above. Examples of preferred alkoxy groups of the invention include methoxy and ethoxy.

In the context of this invention a haloalkoxy group designates an alkoxy group as defined herein, which alkoxy group is substituted one or more times with halo. Preferred haloalkoxy groups of the invention include trihalomethoxy, preferably —OCF₃.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention a cycloalkyl-alkyl group designates a cycloalkyl group as defined above, which cycloalkyl group is substituted on an alkyl group as also defined above. Examples of preferred cycloalkyl-alkyl groups of the invention include cyclopropylmethyl and cyclopropylethyl.

In the context of this invention a cycloalkoxy group designates a “cycloalkyl-O—” group, wherein cycloalkyl is as defined above. Examples of preferred cycloalkoxy groups of the invention include cyclopropylmethoxy and cyclopropylethoxy.

In the context of this invention an aromatic monocyclic or polycyclic heterocyclic group is a mono- or polycyclic compound, which holds one or more heteroatoms in its ring structure. The term “poly-heterocyclic groups” includes benzo-fused five- and six-membered heterocyclic rings containing one or more heteroatoms. Preferred heteroatoms include nitrogen (N), oxygen (O), and sulphur (S).

### Pharmaceutically Acceptable Salts

The aryl piperazine derivatives of the invention may be provided in any form suitable for the intended administration. Suitable forms include pharmaceutically (i.e. physiologically) acceptable salts, and pre- or prodrug forms of the aryl piperazine derivatives of the invention.

Examples of pharmaceutically acceptable salts include, without limitation, the non-toxic inorganic and organic acid salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methane-sulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

### Steric Isomers

Some of the aryl piperazine derivatives of the present invention may exist in (+) and (−) forms as well as in racemic forms (+). The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. A stereo-selective synthetic approach may be pursued. Racemic compounds of the present invention can thus be resolved into their optical antipodes, e.g., by fractional crystallisation of D- or L- (tartrates, mandelates, or camphorsulphonate) salts for example.

Starting materials and/or intermediate compounds used for producing the chemical compounds of the present invention may also be resolved by the formation of diastereomeric amides by reaction of the aryl piperazine derivative of the present invention with an optically active activated carboxylic acid such as that derived from (+) or (−) phenylalanine, (+) or (−) phenylglycine, (+) or (−) camphanic acid or by the formation of diastereomeric carbamates by reaction of the starting material or intermediate compound for use according to the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Methods of Preparation

The aryl piperazine derivatives of the invention may be prepared by conventional methods for chemical synthesis, e.g. those described in the working examples.

Generally amides may be prepared by transforming acids or acid chlorides into the corresponding hydroxy amides by a standard procedure. Esters may be obtained by reacting acidic starting materials with 1,4-dihydroxybutane. After substitution of the terminal hydroxy group by a bromine, hydroxylamides may be treated with the aryl piperazine in the presence of a base to give the desired end product. Compounds based on a ethereal tether may be synthesized starting from the appropriate phenol, which is then condensed with 14-dihydroxybutane or 1,5-dihydroxypentane, followed by transformation into the final products as described above.

Intermediate compounds invention may be resolved by the formation of diastereomeric amides by reaction with an optically active activated carboxylic acid such as that derived from (+) or (−) phenylalanine, (+) or (−) phenylglycine, (+) or (−) camphanic acid or by the formation of diastereomeric carbamates by reaction of the intermediate compound with an optically active chloroformate or the like.

### Biological Activity

The aryl piperazine derivatives of the invention were found to possess selectivity for the dopamine and serotonin receptors, in particular the D₃, D₂-like and 5-HT₂ receptor subtypes. Therefore, in a preferred embodiment, the invention relates to use of the aryl piperazine derivatives of the invention for the treatment, prevention or alleviation of a disease or a disorder or a condition of a mammal, including a human, which disease, disorder or condition is responsive to modulation of the dopamine and serotonin receptors, in particular the D₃, D₂-like and 5-HT₂ receptor subtypes, preferably the dopamine D₃ receptor subtype and/or the D₃/5-HT_{1A} or D₃/5-HT_{2A} receptor subtypes.

In a more preferred embodiment the disease, disorder or condition is a neurological or psychiatric disorders, in particular psychotic disorders, incl. schizophrenia, depression, Parkinson's disease, Huntington's disease, movement disorders, in particular dystonia, anxiety, restlessness, obsessive-compulsive disorders, mania, geriatric disorders, dementia, sexual dysfunction, musculo-skeletal pain symptoms, in particular pain associated with fibromyalgia, sleep disorders, substance abuse or addiction and withdrawal symptoms in drug addicts, cocaine abuse or addiction.

In an even more preferred embodiment the disease, disorder or condition is a neurological or psychiatric disorder, in particular a psychotic disorder, preferably schizophrenia.

In another preferred embodiment the disease, disorder or condition contemplated according to the invention is schizophrenia or Parkinson's disease.

In yet another preferred embodiment the aryl piperazine derivatives of the invention are used as diagnostic tools in diagnostic methods, and in particular for in vivo receptor imaging (neuroimaging).

### Pharmaceutical Compositions

In another aspect the invention provides novel pharmaceutical compositions comprising a therapeutically effective amount of the aryl piperazine derivative of the invention.

While an aryl piperazine derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the aryl piperazine derivative of the invention, or a pharmaceutically acceptable salt or derivative thereof, together with one or more pharmaceutically acceptable carriers, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition of the invention can be prepared by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Mack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the diagnosis, treatment, prevention or alleviation of a disease or a disorder or a condition of a living animal body, including a human, which disease, disorder or condition is responsive to modulation of the dopamine and serotonin receptors, and which method comprises administering to such a living animal body, including a human, in need thereof an effective amount of an aryl piperazine derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Example 1

### Preparatory Example

This example describes synthesis of the compounds presented in Table 1. The compounds may be viewed as having the following generic structure:

### Head-Linker-Tail

TABLE 1
Compounds of the invention

| Cp. No. | Head | Linker | Tail |
|---|---|---|---|
| 1-1 | 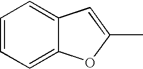 | —CONH(CH₂)₄— | 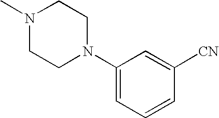 |
| 1-2 | 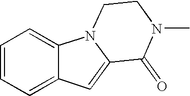 | —(CH₂)₄— | 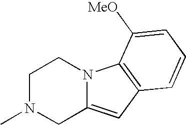 |
| 1-3 | 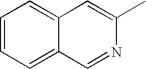 | —CONH(CH₂)₄— | 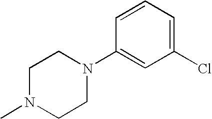 |
| 1-4 | 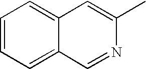 | —CONH(CH₂)₄— | 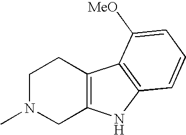 |
| 1-5 | 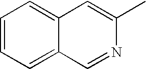 | —CONH(CH₂)₄— | 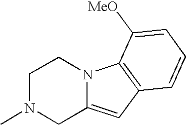 |
| 1-6 | 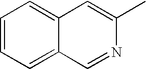 | —O(CH₂)₅— | 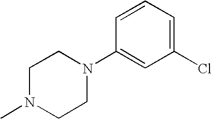 |
| 1-7 | 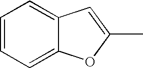 | —CONH(CH₂)₄— | 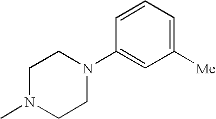 |
| 1-8 | 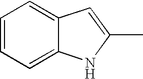 | —COO(CH₂)₄— | 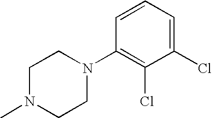 |
| 1-9 | 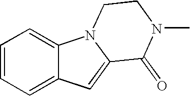 | —(CH₂)₄— | 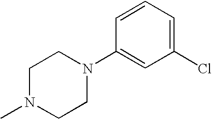 |
| 1-10 | 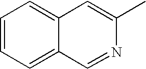 | —CONH(CH₂)₄— | 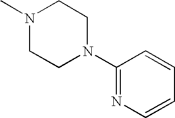 |
| 1-11 | 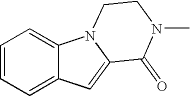 | —(CH₂)₄— | 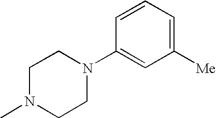 |
| 1-12 | 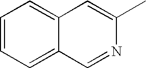 | —O(CH₂)₅— | 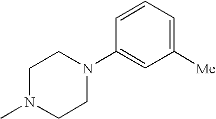 |
| 1-13 | 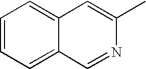 | —CONH(CH₂)₄— | 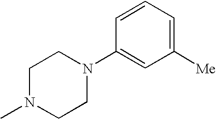 |
| 1-14 | 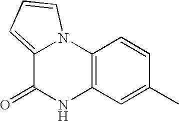 | —O(CH₂)₄— | 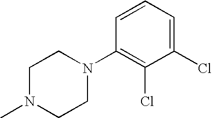 |
| 1-15 | 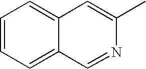 | —CONH(CH₂)₄— | 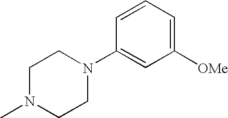 |
| 1-16 | 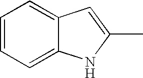 | —CONH(CH₂)₄— | 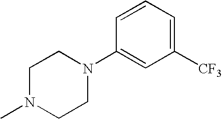 |
| 1-17 | 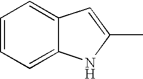 | —(CH₂)₂NHCO(CH₂)₂— | 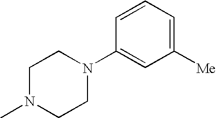 |
| 1-18 | 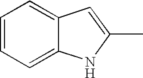 | —(CH₂)₂NHCO(CH₂)₂— | 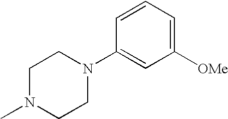 |
| 1-19 | 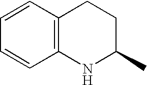 | —CONH(CH₂)₄— | 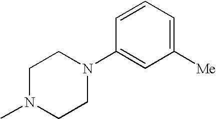 |
| 1-20 | 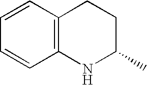 | —CONH(CH₂)₄— | 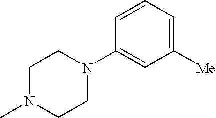 |
| 1-21 | 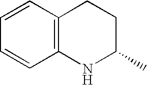 | —CONH(CH₂)₄— | 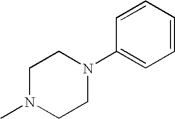 |
| 1-22 | 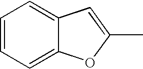 | —CONH(CH₂)₄— | 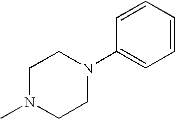 |
| 1-23 | 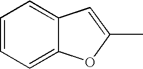 | —CONH(CH₂)₄— | 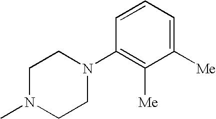 |
| 1-24 | 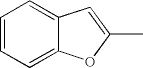 | —CONH(CH₂)₄— | 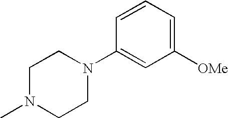 |
| 1-25 | 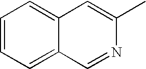 | —CONH(CH₂)₄— | 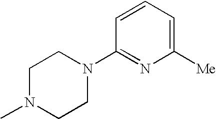 |
| 1-26 | 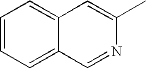 | —CONH(CH₂)₄— | 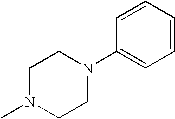 |
| 1-27 | 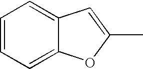 | —CONH(CH₂)₄— | 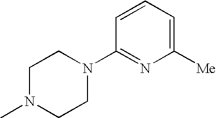 |
| 1-28 | 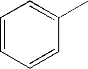 | —CONH(CH₂)₄— | 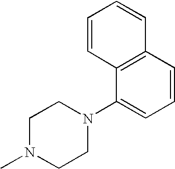 |
| 1-29 | 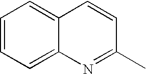 | —CONH(CH₂)₄— | 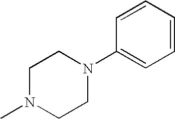 |
| 1-30 | 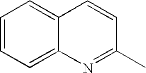 | —CONH(CH₂)₄— | 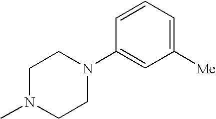 |
| 1-31 | 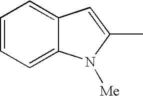 | —CONH(CH₂)₄— | 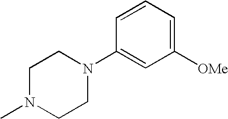 |
| 1-32 | 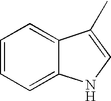 | —CONH(CH₂)₄— | 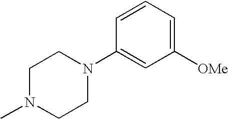 |
| 1-33 | 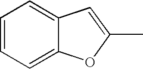 | —CONH(CH₂)₄— | 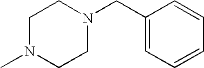 |
| 1-34 | 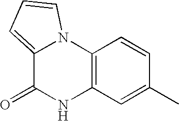 | —O(CH₂)₅— | 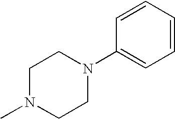 |
| 1-35 | 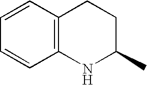 | —CONH(CH₂)₄— | 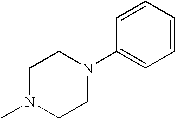 |
| 1-36 | 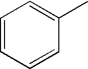 | —CONH(CH₂)₄— | 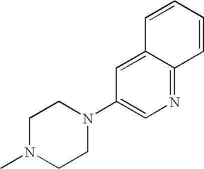 |
| 1-37 | 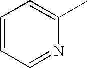 | —CONH(CH₂)₄— | 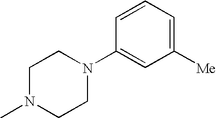 |
| 1-38 | 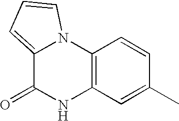 | —O(CH₂)₄— | 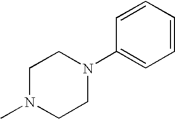 |
| 1-39 | 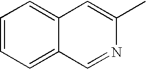 | —CONH(CH₂)₄— | 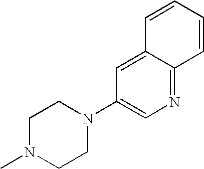 |
| 1-40 | 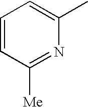 | —CONH(CH₂)₄— | 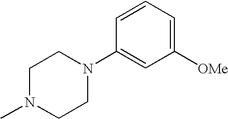 |
| 1-41 | 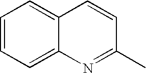 | —CONH(CH₂)₄— | 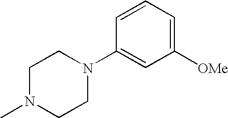 |
| 1-42 | 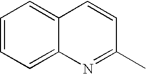 | —CONH(CH₂)₄— | 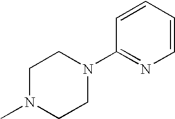 |
| 1-43 | 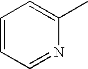 | —CONH(CH₂)₄— | 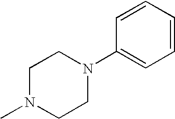 |
| 1-44 | 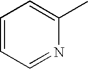 | —CONH(CH₂)₄— | 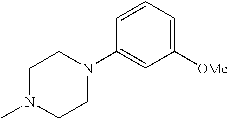 |
| 1-45 | 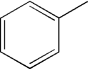 | —CONH(CH₂)₄— | 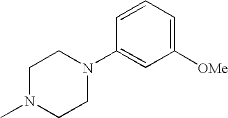 |
| 1-46 | 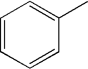 | —CONH(CH₂)₄— | 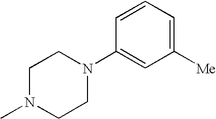 |
| 1-47 | 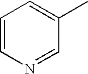 | —CONH(CH₂)₄— | 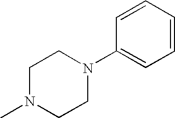 |
| 1-48 | 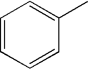 | —CONH(CH₂)₄— | 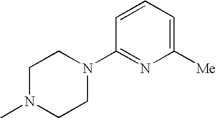 |
| 1-49 | 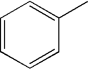 | —CONH(CH₂)₄— | 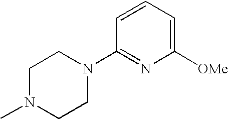 |
| 1-50 | 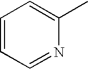 | —CONH(CH₂)₄— | 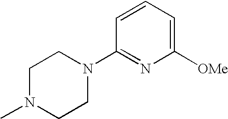 |
| 1-51 | 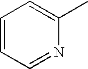 | —CONH(CH₂)₄— | 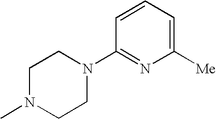 |
| 1-52 | 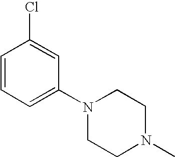 | —(CH₂)₆— | 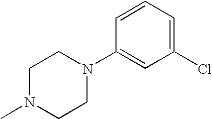 |
| 1-53 | 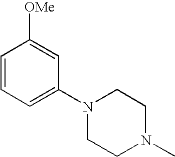 | —(CH₂)₆— | 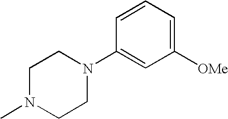 |
| 1-54 | 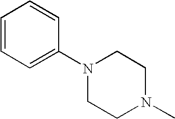 | —(CH₂)₆— | 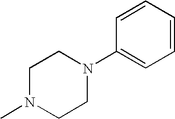 |
| 1-55 | 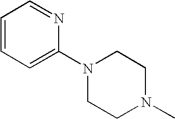 | —(CH₂)₆— | 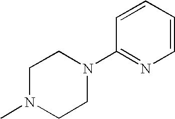 |
| 1-56 | 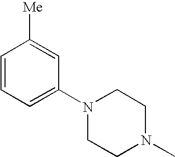 | —(CH₂)₆— | 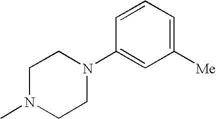 |
| 1-57 | 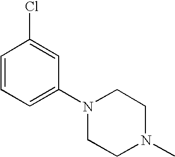 | —(CH₂)₆— | 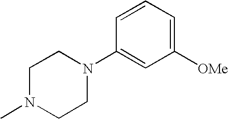 |
| 1-58 | 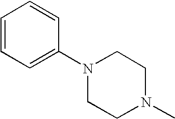 | —(CH₂)₆— | 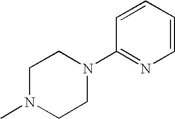 |
| 1-59 | 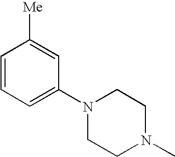 | —(CH₂)₆— | 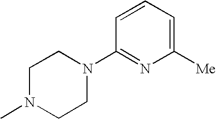 |
| 1-60 | 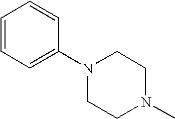 | —(CH₂)₆— | 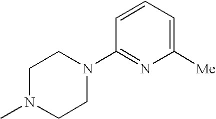 |
| 1-61 | 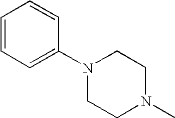 | —(CH₂)₆— | 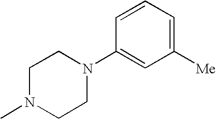 |
| 1-62 | 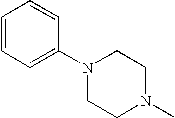 | —(CH₂)₆— | 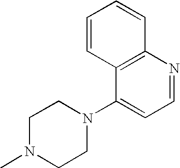 |
| 1-63 | 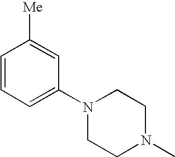 | —(CH₂)₆— | 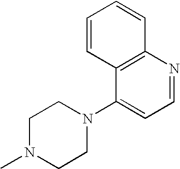 |
| 1-64 | 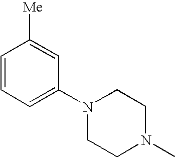 | —(CH₂)₆— | 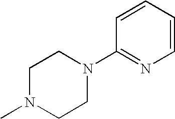 |
| 1-65 | 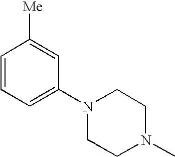 | —(CH₂)₆— | 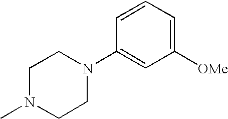 |

### General Description of the Synthesis

Schemes 1-11 outline the route of synthesis following in this example.

Table 2 specifies the variables indicated in the schemes.

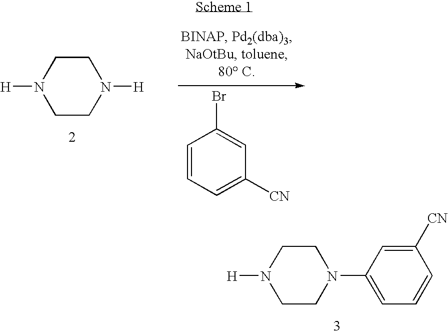

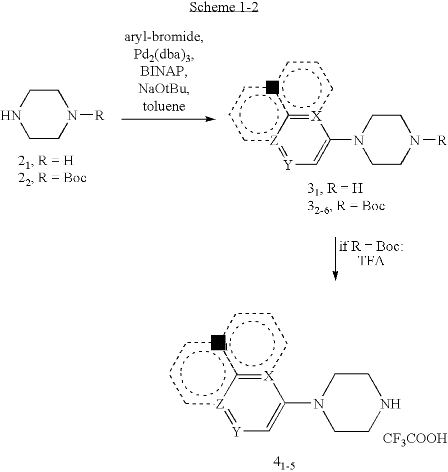

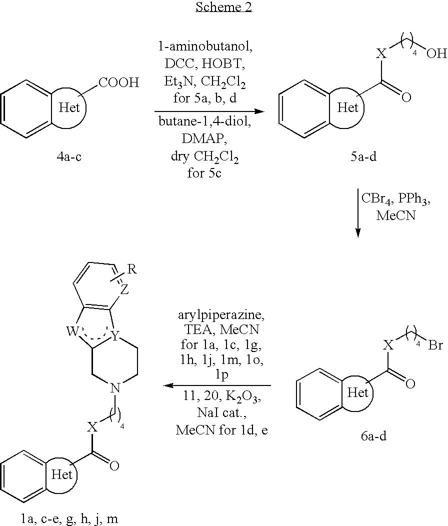

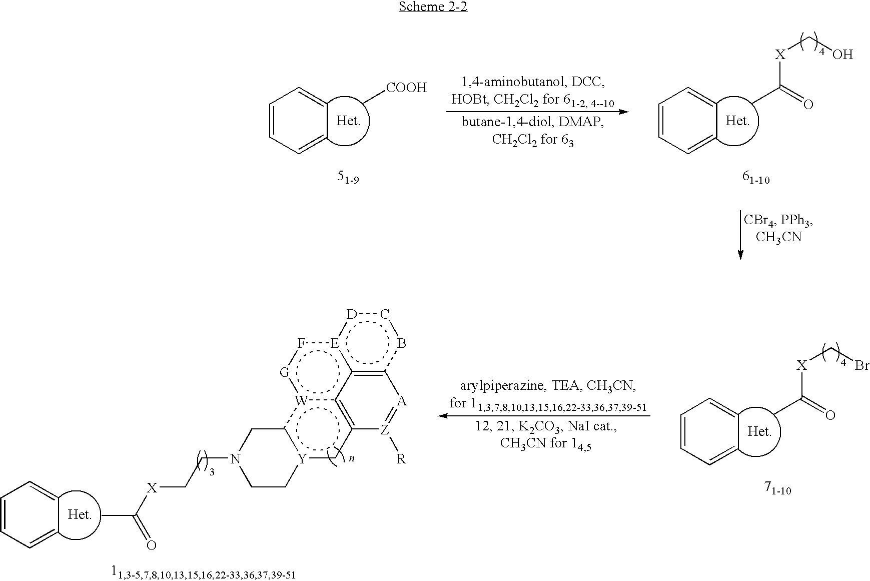

TABLE 2
Specification of variables

| Cp. No. | X | Y | W | Z | R |
|---|---|---|---|---|---|
| 1-1 | NH | N | — | CH | 3-CN |
| 1-3 | NH | N | — | CH | 3-Cl |
| 1-4 | NH | C | NH | CH | 2-OMe |
| 1-5 | NH | N | CH | CH | 2-OMe |
| 1-7 | NH | N | — | CH | 3-Me |
| 1-8 | O | N | — | CH | 2,3-diCl |
| 1-10 | NH | N | — | N | — |
| 1-13 | NH | N | — | CH | 3-Me |
| 1-15 | NH | N | — | CH | 3-OMe |
| 1-16 | NH | N | — | CH | 3-CF₃ |
| 1-22 | NH | N | — | CH | CH |
| 1-23 | NH | N | Me | CH | CH |
| 1-24 | NH | N | — | CH | CH |
| 1-25 | NH | N | — | N | CH |
| 1-26 | NH | N | — | CH | CH |
| 1-27 | NH | N | — | N | CH |
| 1-28 | NH | N | CH | CH | CH |
| 1-29 | NH | N | — | CH | CH |
| 1-30 | NH | N | — | CH | CH |
| 1-31 | NH | N | — | CH | CH |
| 1-32 | NH | N | — | CH | CH |
| 1-33 | NH | N | — | CH | CH |
| 1-36 | NH | N | — | CH | N |
| 1-37 | NH | N | — | CH | CH |
| 1-39 | NH | N | — | CH | N |
| 1-40 | NH | N | — | CH | CH |
| 1-41 | NH | N | — | CH | CH |
| 1-42 | NH | N | — | N | CH |
| 1-43 | NH | N | — | CH | CH |
| 1-44 | NH | N | — | CH | CH |
| 1-45 | NH | N | — | CH | CH |
| 1-46 | NH | N | — | CH | CH |
| 1-47 | NH | N | — | N | CH |
| 1-48 | NH | N | — | N | CH |
| 1-49 | NH | N | — | N | CH |
| 1-50 | NH | N | — | CH | CH |
| 1-51 | NH | N | — | CH | CH |

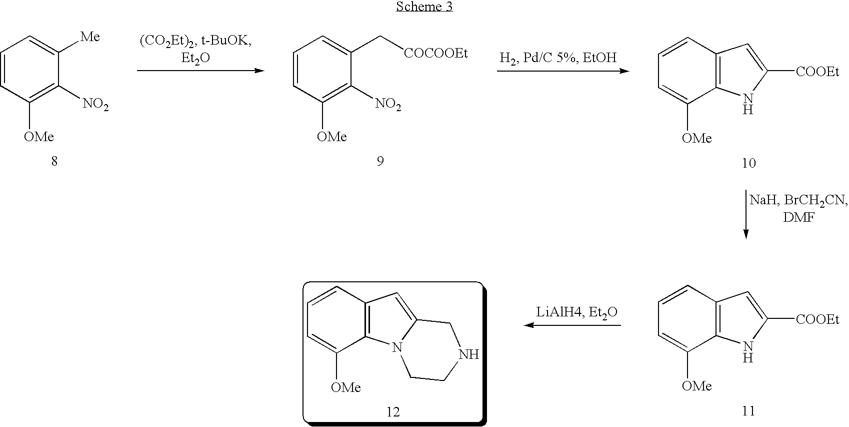

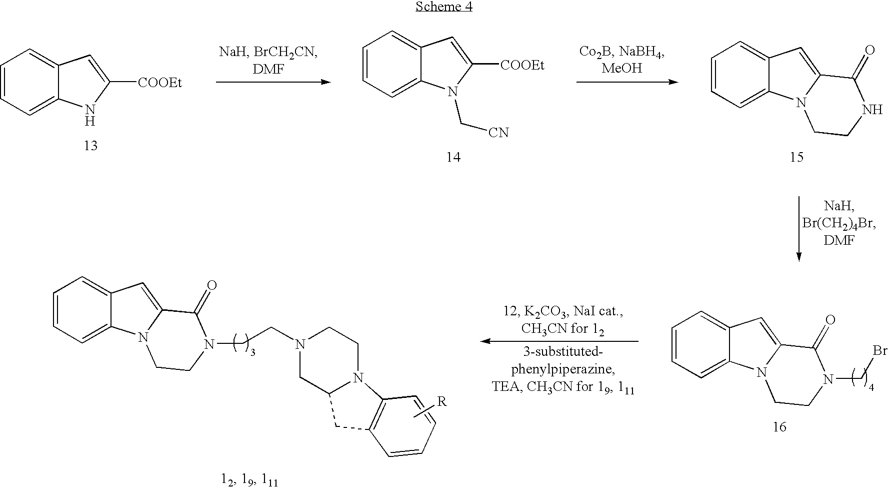

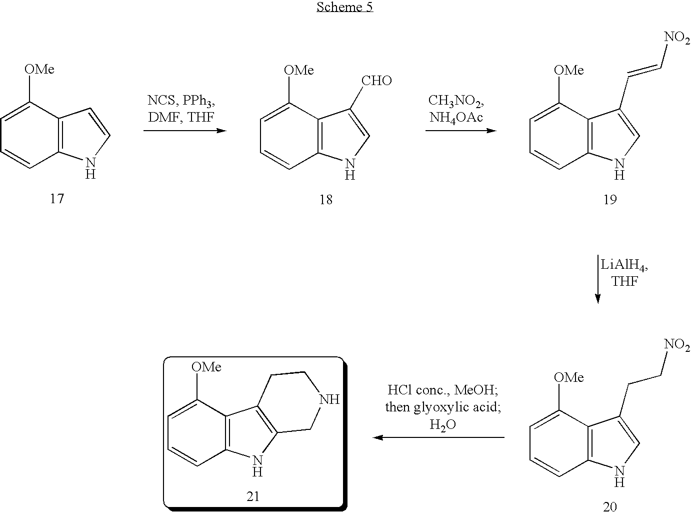

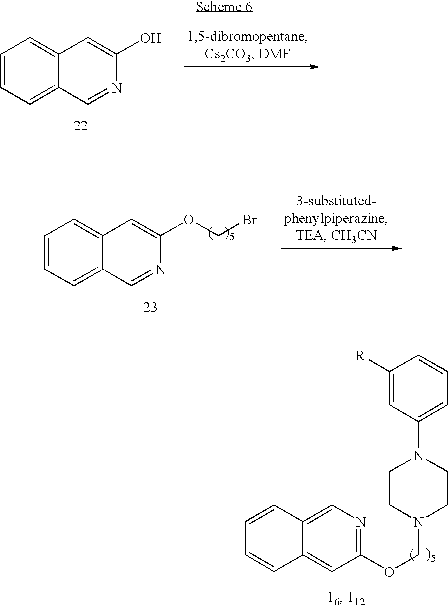

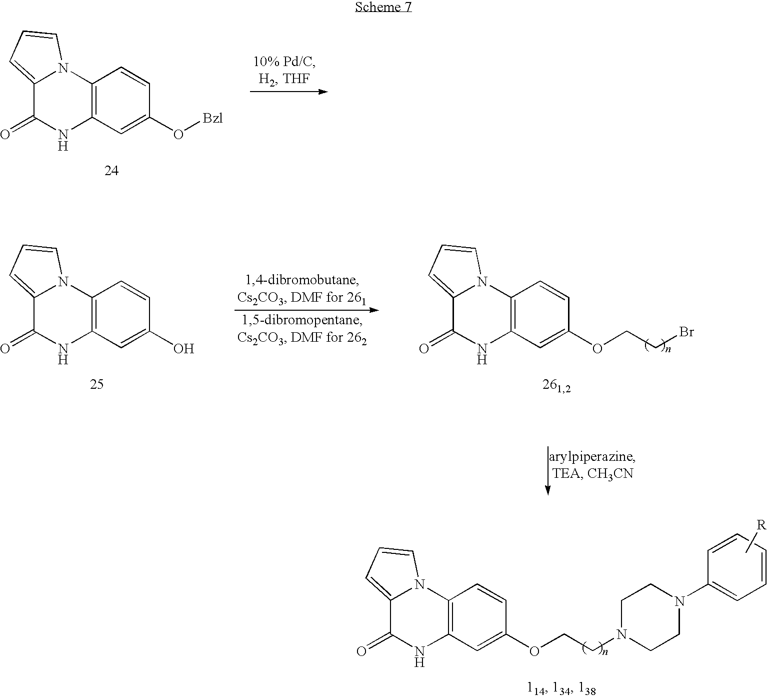

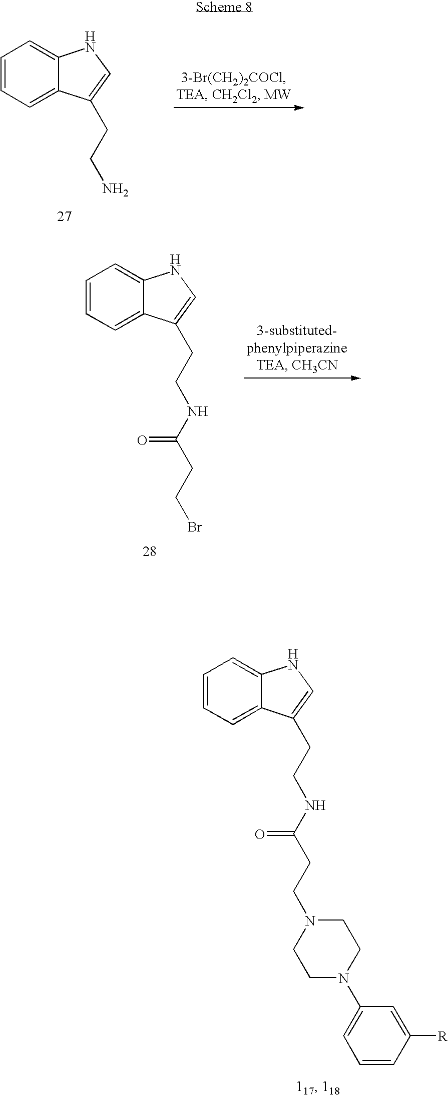

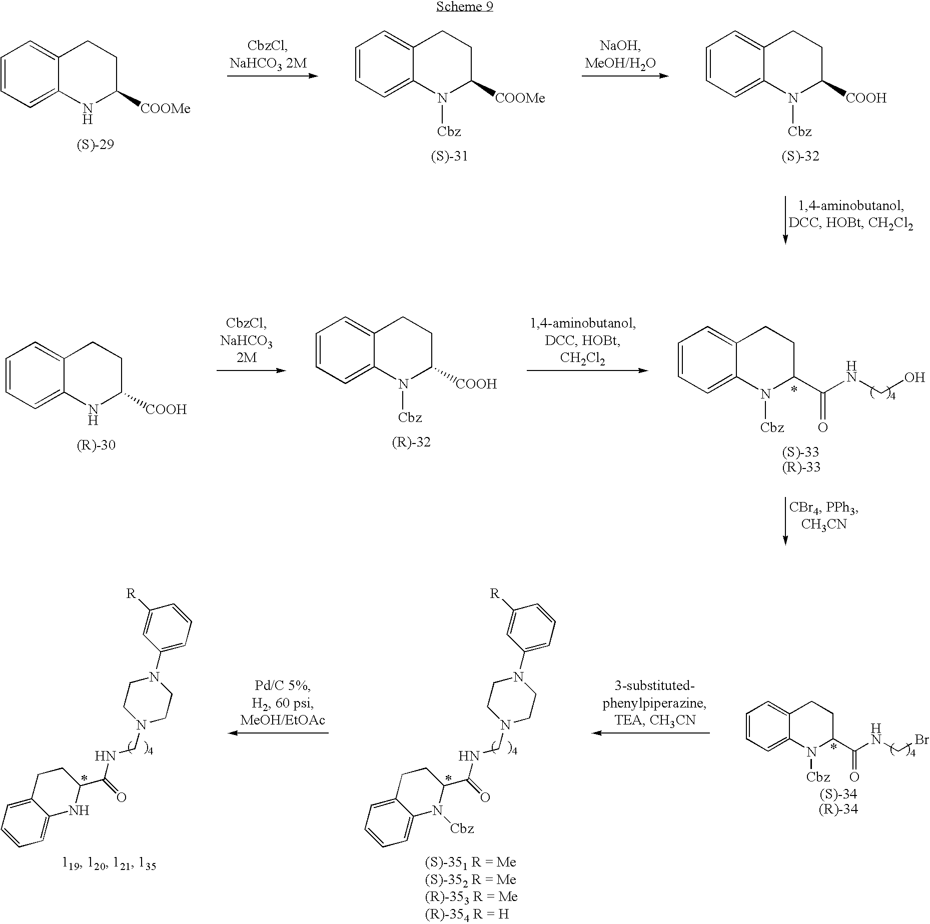

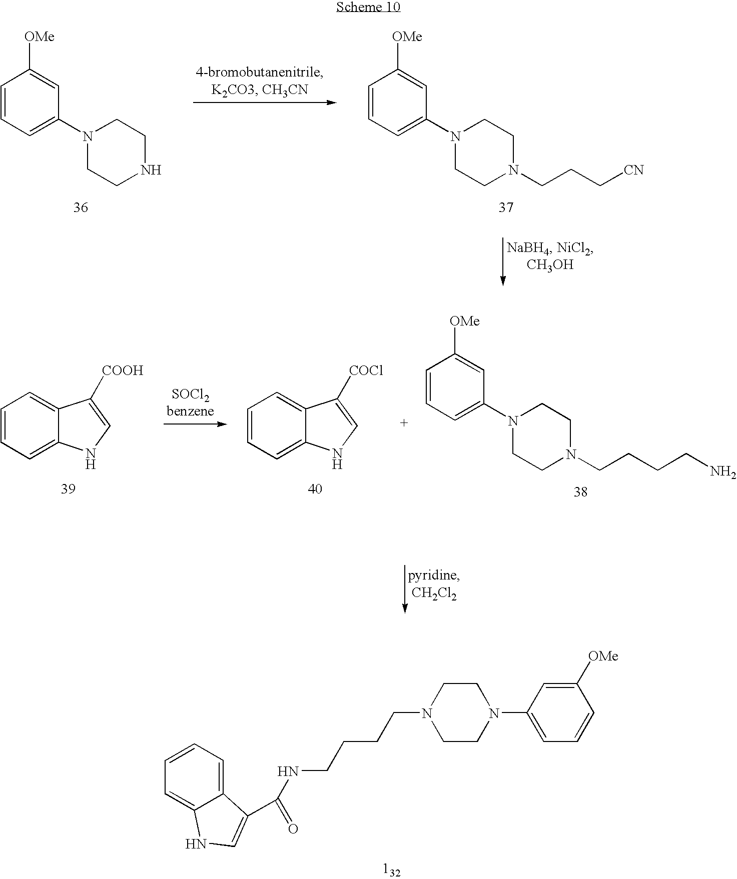

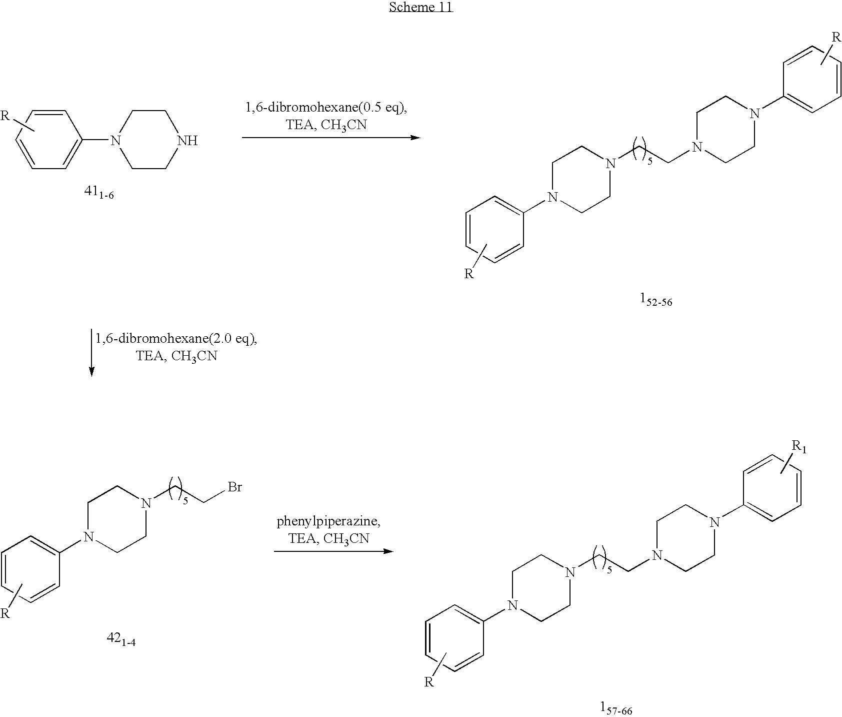

Melting points were determined using an Electrothermal 8103 apparatus. IR spectra were taken with Perkin-Elmer 398 and FT 1600 spectrophotometers. ¹H NMR spectra were recorded on a Bruker 200 MHz spectrometer with TMS as internal standard; the value of chemical shifts (δ) are given in ppm and coupling constants (J) in Hertz (Hz). All reactions were carried out in an argon atmosphere. GC-MS were performed on a Saturn 3 (Varian) or Saturn 2000 (Varian) GC-MS System using a Chrompack DB5 capillary column (30 m×0.25 mm i. d.; 0.25 μm film thickness). Mass spectra were recorded using a VG 70-250S spectrometer. ESI-MS and APCI-MS spectra were taken by a LCQDeca-Thermofinnigan spectrometer. Optical rotations were recorded on a Perkin-Elmer Model 343 polarimeter at the sodium D line at 20° C. Elemental analyses were done on a Perkin-Elmer 240C elemental analyser and the results were within 0.4% of the theoretical values, unless otherwise noted. Yields refer to purified products and are not optimised. For testing, the claimed compounds were transformed into the corresponding hydrochloride salts by a standard procedure.

### N-[4-[4-(3-Cyanophenyl)piperazin-1-yl]butyl]benzo[b]furan-2-carboxamide (Compound 1-1/1a)

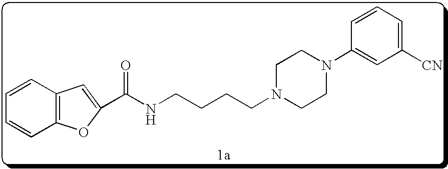

1-(3-Cyanophenyl)piperazine (3). A mixture of 3-bromobenzonitrile (0.50 g, 2.74 mmol), piperazine (0.71 g, 8.24 mmol), sodium tert-butoxide (0.37 g. 3.8 mmol), tris(dibenzylideneacetone)dipalladium-(0) (6.27 mg, 0.0068 mmol) and rac-2,2′-bis(diphenylphosphino)-1,1′-binaphthyl (BINAP) in dry toluene (8.0 mL) was heated to 80° C. under argon. After stirring for 2 h, the mixture was allowed to cool to room temperature, taken up in ethyl ether (30.0 mL), filtered and concentrated. The crude product was then purified by means of flash chromatography (10% methanol in chloroform) to give 0.32 g (63% yield) of 3 as a yellow oil: ¹H NMR (CDCl₃) δ 1.87 (br s, 1H), 3.03 (t, 4H; J=4.4 Hz), 3.17 (t, 4H; J=4.3 Hz), 7.08 (m, 3H), 7.31 (m, 1H); IR (CHCl₃) Vₘₐₓ 2230 cm⁻¹. Anal. (C₁₁H₁₃N₃) C, H, N.

N-[4-(1-Hydroxy)butyl]benzo[b]furan-2-carboxamide (5a). To a solution of 2-benzofurancarboxylic acid 4a (0.50 g, 3.08 mmol) in dry dichloromethane (10.0 mL), 1-hydroxybenzotriazole hydrate (HOBT) (0.46 g, 3.40 mmol) and 1,3-dicyclohexylcarbodiimide (0.70 g, 3.40 mmol) were added at 0° C. under argon; the suspension was warmed to room temperature and stirred for 1 h. Then 4-amino-1-butanol (0.28 mL, 3.08 mmol) was added and the mixture was stirred overnight at room temperature. The resulting suspension was filtered through Celite®, washed with chloroform (3×10 mL) and the filtrate evaporated. The crude product was purified by means of flash chromatography (10% methanol in chloroform) to give 0.70 g (97%) of 5a as a white solid: mp (methanol) 95-96° C.; ¹H NMR (CDCl₃) δ 1.67 (m, 4H), 2.14 (br s, 1H), 3.53 (m, 2H), 3.73 (m, 2H), 6.89 (br s, 1H), 7.25-7.48 (m, 4H), 7.63 (d, 1H; J=7.7 Hz). Anal. (C₁₃H₁₅NO₃) C, H, N.

N-[4-(1-Bromo)butyl]benzo[b]furan-2-carboxamide (6a). To a vigorous stirred solution of 5a (0.50 g, 2.14 mmol) in dry acetonitrile (25.0 mL), triphenylphosphine (0.86 g, 3.22 mmol) and carbon tetrabromide (1.06 g, 3.22 mmol) were added at room temperature. After 2 h the mixture was quenched with 15% NaOH and the heterogeneous mixture was extracted with ethyl acetate (EtOAc) (3×25 mL). The organic layers were dried and evaporated. The residue was chromatographed (20% n-hexane in ethyl acetate) to afford 0.58 g (91% yield) of 6a as white solid: mp (EtOAc) 65-66° C.; ¹H NMR (CDCl₃) δ 1.67 (m, 4H), 3.37 (m, 4H), 7.36 (m, 4H), 7.63 (d, 1H; J=7.7 Hz); GC-MS m/z 297 [M+H]⁺, 216 (100), 202, 188, 174, 161, 145, 118, 89. Anal. (C₁₃H₁₄BrNO₂) C, H, N.

N-[4-[4-(3-Cyanophenyl)piperazin-1-yl]butyl]benzo[b]furan-2-carboxamide (1-1/1a). To a stirred solution of 6a (50.0 mg, 0.17 mmol) in dry acetonitrile (3.0 mL) under argon, 1-(3-cyanophenyl)piperazine 3 (31.7 mg, 0.17 mmol) and triethylamine (38.2 μL, 0.27 mmol) were added; the solution was refluxed overnight under stirring. The solvent was removed under reduced pressure, water was added and the mixture was extracted with dichloromethane (3×10 mL). The organic layers were dried and concentrated and the crude product was chromatographed (10% methanol in chloroform) to give 60.0 mg of 1a (90% yield) as colorless oil. ¹H NMR (CDCl₃) δ 1.72 (m, 4H), 2.46 (t, 2H, J=6.7 Hz), 2.61 (t, 4H, J=4.9 Hz), 3.24 (t, 4, J=5.0 Hz), 3.52 (q, 2H, J=6.1 Hz), 6.89 (br s, 1H), 7.09 (m, 3H), 7.47-7.25 (m, 5H), 7.66 (d, 1H, J=7.5); FAB-MS m/z 403 [M+H]⁺, 147. Anal. (C₂₄H₂₆N₄O₂) C, H, N.

### 3,4-Dihydro-2-[4-(3,4-dihydro-6-methoxypyrazino[1,2-a]indol-2(1H)-yl)butyl]pyrazino[1,2-a]indol-1(2H)-one (Compound 1-2/1 b)

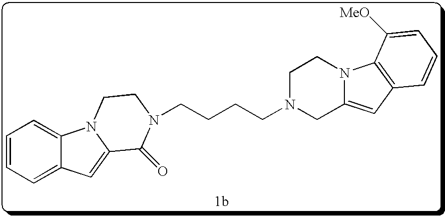

Ethyl 3-(3-methoxy-2-nitrophenyl)-2-oxopropanoate (8). To a suspension of potassium tert-butoxide (2.0 g, 18.0 mmol) in dry diethyl ether (50.0 mL), diethyl oxalate (3.16 mL, 23.3 mmol) was added dropwise at room temperature under and the mixture was stirred for 15 min. Then 3-methoxy-2-nitrotoluene 7 (3.0 g, 18.0 mmol) was added and the mixture was stirred for 30 min. and left 12 h without stirring. The solvent was removed in vacuo and water and solid ammonium chloride were added to the residue. The aqueous mixture was extracted with ethyl acetate (3×25 mL) and the collected organic layer were dried over anhydrous sodium sulfate (Na₂SO₄) and the solvent was evaporated. The crude product was chromatographed (30% EtOAc in n-hexane) to give 4.2 g of pure 8 as yellow oil 88% yield; ¹H NMR (CDCl₃) δ 1.27 (m, 3H), 2.18 (s, 3H), 3.75 (m, 2H), 4.25 (m, 2H), 6.78 (m, 2H), 7.20 (m, 1H); GC-MS m/z 267 [M]⁺ 194, 166 (100), 135, 121; ES-MS m/z 268 [M+H]⁺. Anal. (C₁₂H₁₃NO₆) C, H, N.

Ethyl 7-methoxy-1H-indole-2-carboxylate (9). To a solution of 8 (4.8 g, 18.0 mmol) in absolute ethanol (120.0 mL) previously degassed under N₂, catalytic 5% Pd/C was added and the mixture was allowed under hydrogen atmosphere at room temperature for 24 h. The mixture was filtered over Celite® washing with ethanol and the filtrate was evaporated under reduced pressure. The crude product was purified by means of flash chromatography (20% EtOAc in n-hexane) to afford 9 as yellow solid (79%): mp 69-72° C.; ¹H NMR (CDCl₃) δ 1.42 (t, 3H, J=6.94 Hz), 3.96 (s, 3H), 4.41 (q, 2H, J=7.2 Hz), 6.72 (d, 1H, J=7.5 Hz), 7.07 (t, 1H, J=7.9 Hz), 7.25 (m, 2H), 9.18 (br s, 1H). ESI-MS m/z 220 [M+H]⁺, ES-MS/MS of [M+H]⁺ 192, 176, 174 (100), 176, 148. Anal. (C₁₂H₁₃NO₃) C, H, N.

Ethyl 1-(cyanomethyl)-7-methoxy-1H-indol-2-carboxylate (10). A mixture of sodium hydride (60% dispersion in mineral oil, 509.6 mg, 21.23 mmol) and ethyl 7-methoxyindole-2-carboxylate 9 (3.1 g, 14.15 mmol) in dry N,N-dimethylformamide (DMF) (15.0 mL), was stirred at room temperature for 30 min and to this bromoacetonitrile (2.0 mL, 28.3 mmol) in dry DMF (2.0 mL) was added. The reaction mixture was then maintained at 60-65° C. for 30 min, and stirred for further 6 h at room temperature, left overnight and decomposed with ice. The separated solid was filtered and purified by means of flash chromatography (33% n-hexane in dichloromethane) to give 10 (30% yield) as white solid: mp (ethanol) 99-101° C.; ¹H NMR (CDCl₃) δ 1.41 (t, 3H J=7.2 Hz), 3.99 (s, 3H), 4.40 (q, 2H, J=7.1 Hz), 5.96 (s, 2H), 6.80 (d, 1H, J=7.7 Hz), 7.10 (t, 1H, J=7.9 Hz), 7.25 m, 1H), 7.33 (s, 1H). GC-MS m/z 258 (100) [M]⁺, 232, 213, 201, 187, 172, 144, 130, 114, 89. Anal. (C₁₄H₁₄N₂O₃) C, H, N.

1,2,3,4-Tetrahydro-4-methoxypyrazino[1,2-a]indole (11). A suspension of 10 (0.50 g, 1.93 mmol) in dry diethyl ether (Et₂O) (20.0 mL) was added slowly to a well-stirred slurry of lithiumaluminium hydride (LiAlH₄) (293.4 mg, 7.72 mmol) in dry Et₂O (10.0 mL). The mixture was refluxed for 8 h. The reaction mixture was poured into ice-water bath and 1N NaOH (10.0 mL) was added. The aqueous phase was extracted with EtOAc (3×30 mL) and the collected organic layers were dried and evaporated. The crude product was chromatographed (10% methanol in chloroform) to afford 11 as yellow solid (40% yield): mp 120-122° C.; ¹H NMR (CDCl₃) δ 7.14 (d, 1H, J=7.8 Hz), 6.97 (t, 1H, J=7.7 Hz), 6.58 (d, 1H, J=7.7 Hz), 6.14 (s, 1H), 4.47 (t, 2H, J=5.8 Hz), 4.17 (s, 2H), 3.90 (s, 3H), 3.26 (t, 2H, J=5.7 Hz); ES-MS m/z 405 [2M+H]⁺, 203 (100) [M+H]⁺. Anal. (C₁₂H₁₄N₂O) C, H, N.

Ethyl 1-(cyanomethyl)-1H-indol-2-carboxylate (13). A mixture of sodium hydride (60% dispersion in mineral oil, 190.0 mg, 7.94 mmol) and ethyl indole-2-carboxylate 12 (1.0 g, 5.29 mmol) in dry DMF (4.6 mL), was stirred at room temperature for 30 min and to this bromoacetonitrile (0.74 mL, 10.60 mmol) in dry DMF (1.0 mL) was added. The reaction mixture was then maintained at 65° C. for 30 min, and stirred for further 6 h at room temperature, left overnight and decomposed with ice. The separated solid was re-crystallized from ethanol to give 13 (90% yield) as white solid: mp (ethanol) 83-84° C.; ¹H NMR (CDCl₃) δ 1.42 (t, 3H; J=7.3 Hz), 4.41 (q, 2H; J=14.2, 7.2 Hz), 5.60 (s, 2H), 7.37 (m, 4H), 7.71 (d, 1H; J=7.9 Hz); GC-MS m/z 228 [M]⁺ (100), 199, 182, 154, 128, 115, 101, 89, 77. Anal. (C₁₃H₁₂N₂O₂) C, H, N.

1,2,3,4-Tetrahydropyrazino(2H)-1-one[1,2-a]indole (14). To a warm solution (60° C.) of 13 (200.0 mg, 0.87 mmol) in dry methanol (8.0 mL) under argon, freshly prepared cobalt boride (450.0, 3.50 mmol), was added under stirring. Sodium borohydride (166.0 mg, 4.38 mmol) was cautiously added portionwise and the mixture was refluxed for 3 h. The mixture was cooled, and the solvent removed under reduced pressure then water was added and the mixture was extracted with EtOAc (3×25 mL). The organic layers were dried, evaporated and the crude product was purified by means of flash chromatography (10% methanol in chloroform) to give 14 (68% yield) as white solid: mp (methanol) 261-265° C. (dec.); ¹H NMR (CDCl₃) δ 3.82 (m, 2H), 4.27 (m, 2H), 6.65 (br s, 1H), 7.23 (m, 4H), 7.72 (d, 1H; J=8.0 Hz); APCI-MS m/z 187 [M+H]⁺; APCI-MS/MS of [M+H]⁺ 159 (100), 144. Anal. (C₁₁H₁₀N₂O) C, H, N.

N-[1-(4-Bromo)butyl]-1,2,3,4-tetrahydropyrazino(2H)-1-one[1,2-a]indole (15). To a suspension of 14 (130.0 mg, 0.69) dry DMF (1.0 mL) sodium hydride, 60% in mineral oil, (20.0 mg, 0.83 mmol) was added. After stirring for 1 h at 60° C. under argon, a solution of 1,4-dibromobutane (0.41 mL, 3.47 mmol) in dry DMF (0.50 mL) was added dropwise. The mixture was refluxed under argon at 110° C. for 3 h. Then the solvent was evaporated under reduced pressure, and the residue was re-suspended in water and extracted with dichloromethane (3×10 mL). The combined organic layers were dried evaporated and the residue was chromatographed (30% EtOAc in n-hexane) to give 15 as yellow solid (41% yield): mp (EtOAc) 101-102° C.; ¹H NMR (CDCl₃) δ δ 1.85 (m, 4H), 3.67 (m, 4H), 3.81 (m, 2H), 4.29 (m, 2H), 7.20 (m, 4H), 7.70 (d, 1H; J=8.0 Hz); APCI-MS m/z 321 [M+H]⁺, 241, 227, 199 (100), 187, 159, 144, 117. Anal. (C₁₅H₁₇BrN₂O) C, H, N.

3,4-Dihydro-2-[4-(3,4-dihydro-6-methoxypyrazino[1,2-a]indol-2(1H)-yl)butyl]pyrazino[1,2-a]indol-1(2H)-one (1b). To a suspension of 1,2,3,4-tetrahydro-6-methoxypyrazino[1,2-a]indole 11 (30.0 mg, 0.15 mmol) and K₂CO₃ (71.6 mg, 0.52 mol) in dry acetonitrile (5.0 mL) bromo-derivative 15 (47.7 mg, 0.15 mmol) and a catalytic amount of sodium iodide (NaI) were added and the resulting mixture was heated at reflux for 18 h. Then the mixture was filtered and the filtrate was evaporated to dryness under reduced pressure. The residue was suspended in water (10.0 mL) and extracted with Et₂O (2×25 mL). The combined ethereal extract was evaporated under reduced pressure and the crude product was purified by means of flash chromatography (5% methanol in chloroform) affording to 1b as yellow oil (62% yield); ¹H NMR (CDCl₃) δ 1.69 (m, 4H), 2.58 (t, 2H, J=6.6 Hz), 2.88 (t, 2H, J=5.5 Hz), 3.66 (t, 2H, J=6.7 Hz), 3.77 (m, 4H), 3.88 (s, 3H), 4.24 (t, 2H, J=5.8 Hz), 4.45 (t, 2H, J=5.6 Hz), 6.10 (s, 1H), 6.54 (d, 1H, J=7.6 Hz), 6.93 (t, 1H, J=7.8 Hz), 7.13 (m, 3H), 7.29 (m, 2H), 7.70 (d, 1H, J=7.9 Hz); ES-MS m/z 907 [2M+Na]⁺, 884 [2M+H]⁺, 443 (100) [M+H]⁺; ¹³C NMR (CDCl₃) δ 24.3, 25.3, 29.6, 40.2, 45.5, 45.9, 46.1, 51.2, 51.7, 55.3, 57.2, 97.1, 101.8, 106.0, 109.5, 112.9, 120.6, 122.6, 124.3, 125.9, 127.5, 129.4, 130.3, 134.5, 136.3, 147.7, 159.9. Anal. (C₂₇H₃₀N₄O₂) C, H, N.

### N-[4-[4-(3-Chlorophenyl)piperazin-1-yl]butyl]isoquinoline-3-carboxamide (Compound 1-3/1c)

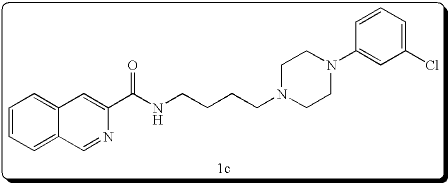

N-[1-(4-Hydroxy)butyl]isoquinolin-3-carboxamide (5b). The title compound was prepared starting from 3-isoquinolinecarboxylic acid 4b (100.0 mg, 0.57 mmol) and following the procedure as described to obtain 5a. The derivative 5b was obtained as white solid (96% yield): mp (methanol) 126-127° C.; ¹H NMR (CDCl₃) δ 1.74 (m, 4H), 3.57 (q, 2H; J=6.3 Hz), 3.73 (t, 2H; J=5.9 Hz), 7.40 (m, 2H), 7.75 (m, 2H), 8.39 (br s, 1H), 8.57 (s, 1H), 9.14 (s, 1H); ES-MS m/z 510 [2M+Na]⁺, 267 (100) [M+Na]⁺, 245 [M+H]⁺, ES-MS/MS of [M+H]⁺ 227, 174. Anal. (C₁₄H₁₆N₂O₂) C, H, N.

N-[1-(4-Bromo)butyl]isoquinolin-3-carboxamide (6b). To a solution of 5b (140.0 mg, 0.57 mmol) in dry acetonitrile (10.0 mL) triphenylphosphine (225.0 mg, 0.86 mmol) and carbon tetrabromide (285.0 mg, 0.86 mmol) were added under vigorous stirring at room temperature. After 2 h the mixture was quenched with 15% NaOH and extracted with EtOAc (3×10 mL). The organic layers were dried and evaporated. The residue was chromatographed (30% n-hexane in EtOAc) to give 130.0 mg of 6b (75% yield) as yellow solid: mp (EtOAc) 72-73° C.; ¹H NMR (CDCl₃) δ 2.06 (m, 4H), 3.48 (m, 4H), 7.66 (m, 2H), 7.93 (m, 2H), 8.36 (br s, 1H), 8.55 (s, 1H), 9.08 (s, 1H); ES-MS m/z 329 [M+Na]⁺, 308 (100) [M+Na]⁺. Anal. (C₁₄H₁₅BrN₂O) C, H, N.

N-[4-[4-(3-Chlorophenyl)piperazin-1-yl]butyl]isoquinoline-3-carboxamide (1c). To a stirred solution of 6b (190.0 mg, 0.62 mmol) in dry acetonitrile (20.0 mL) under argon, 1-(3-chlorophenyl)piperazine hydrochloride (144.0 mg, 0.62 mmol) and triethylamine (141.0 μL, 1.0 mmol) were added; the solution was refluxed overnight under stirring. The solvent was removed under reduced pressure, water was added and the mixture was extracted with dichloromethane (3×10 mL). The organic layers were dried and concentrated and the crude product was chromatographed (10% methanol in chloroform) to give 130.0 mg of 1c (50% yield) as white solid: mp (methanol) 156-157° C.; ¹H NMR (CDCl₃) δ 1.65 (m, 4H), 2.46 (t, 2H, J=6.7 Hz), 2.60 (t, 4H, J=4.9 Hz), 3.21 (t, 4H, J=5.0 Hz), 3.57 (q, 2H, J=6.5 Hz), 6.78 (m, 2H), 6.86 (d, 1H, J=1.6 Hz), 7.14 (t, 1H, J=8.0 Hz), 7.72 (m, 2H), 8.00 (t, 2H, J=8.2 Hz), 8.33 (br s, 1H), 8.61 (s, 1H), 9.14 (s, 1H); ES-MS m/z 445 (100) [M+Na]⁺, 423 [M+H]⁺, ES-MS/MS of [M+H]⁺ 251, 227 (100); ¹³C NMR (CDCl₃) δ 24.4, 27.8, 29.8, 39.5, 48.7, 53.1, 58.2, 114.0, 115.9, 119.4, 120.4, 127.8, 128.3, 128.9, 129.8, 130.2, 131.2, 135.1, 136.2, 144.0, 151.2, 152.5, 165.0. Anal. (C₂₄H₂₇ClN₄O) C, H, N.

### N-[4-(1,2,3,4-Tetrahydro-5-methoxy-β-carbolin-2-yl)butyl]isoquinoline-3-carboxamide (Compound 1-4/1d)

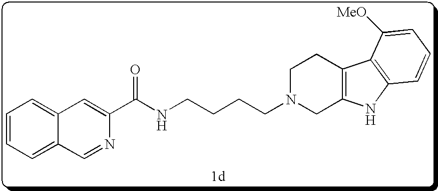

4-Methoxy-1H-indole-3-carbaldehyde (17). N-Chlorosuccinimide (2.72 g, 20.41 mmol) was added portionwise to a solution of triphenylphosphine (5.35 g, 20.41 mmol) in tetrahydrofuran (100.0 mL) and stirred at room temperature for 30 min. DMF (1.54 mL, 40.8 mmol) was added to the suspension, and the mixture was heated to reflux for 1 h. Then 4-methoxy-1H-indole 16 (1.0 g, 6.80 mmol) was added and the mixture was heated to reflux for 1 h. After cooling, tetrahydrofuran was evaporated, water (80.0 mL) added, and the mixture heated to reflux for 1 h and then alkalinized with 10% NaOH. After the aqueous phase was extracted with EtOAc (3×50 mL) and the collected organic layers were dried and evaporated. The crude product was purified by means of flash chromatography (50% n-hexane in EtOAc) to give 17 as orange solid in quantitative yield: mp (EtOAc) 154-156° C.; ¹H NMR (CDCl₃) δ 4.00 (s, 3H), 6.72 (m, 1H), 7.07 (d, 1H, J=8.1 Hz), 7.19 (d, 1H, J=8.1 Hz), 7.92 (d, 1H, J=2.9 Hz), 9.05 (br s, 1H), 10.50 (s, 1H); FAB-MS m/z 175 (100) [M]⁺, 160, 144, 129, 116, 104, 89, 77. Anal. (C₁₀H₉NO₂) C, H, N.

4-Methoxy-3-(2-nitrovinyl)-1H-indole (18). Ammonium acetate (168.0 mg, 2.19 mmol), was added to 4-methoxy-1H-indole-3-carbaldehyde 17 (1.20 g, 7.27 mmol) in nitromethane (12.0 mL) and the mixture was stirred vigorously while heating to reflux for 1 h. The resulting solution was concentrated under reduced pressure and the crude product was purified by means of flash chromatography (50% n-hexane in EtOAc) to give 0.95 g of 18 as bright-yellow solid (64% yield): mp (EtOAc) 179-181° C. dec.; ¹H NMR (DMSO-_{d6}) δ 3.95 (s, 3H), 6.73 (d, 1H, J=7.3 Hz), 7.13 (m, 2H), 8.08 (d, 1H, J=13.4 Hz), 8.24 (s, 1H), 8.55 (d, 1H, J=13.0 Hz), 12.20 (br s, 1H). Anal. (C₁₁H₁₀N₂O₃) C, H, N.

4-Methoxytryptamine (19). A solution of 18 (0.90 g, 4.33 mmol) in dry tetrahydrofuran (40.0 mL) was added dropwise to a suspension of LiAlH₄ (1.73 g, 45.22 mmol) in dry tetrahydrofuran (17.0 mL) and heated under reflux for 1 h with stirring. Excess of LiAlH₄ was quenched by addiction of methanol with caution under cooling in an ice bath. Then water and s.s. sodium-potassium tartrate were added and the mixture was extracted with a solution of dichloromethane-methanol (95:5 v/v). The organic layers were washed with brine, dried over Na₂SO₄, and evaporated under reduced pressure. The crude product was purified by means of flash chromatography (CHCl₃-MeOH—NH₄OH 20:5:1 v/v) to afford 0.60 g of 19 as white solid (77% yield: mp (methanol) 139-140° C.; ¹H NMR (CDCl₃) δ 2.00 (br s, 2H), 3.00 (m, 4H), 3.89 (s, 3H), 6.46 (d, 1H, J=7.6 Hz), 6.80 (s, 1H), 6.92 (d, 1H, J=8.1 Hz), 7.06 (t, 1H, J=7.9 Hz), 8.85 (br s, 1H). Anal. (C₁₁H₁₄N₂O) C, H, N.

1,2,3,4-Tetrahydro-5-methoxy-β-carboline (20). 4-Methoxytryptamine 19 (375.0 mg, 1.97 mmol) was previously transformed in the corresponding hydrochloride salt by standard procedure. To a solution of 4-methoxytryptamine hydrochloride (445.0 mg, 1.97 mmol) in water (50.0 mL) glyoxylic acid monohydrate (181.2 mg, 1.97 mmol) was added and the mixture was stirred at reflux for 1 h. After cooling at room temperature a solution of 20% NaOH was added and the mixture was extracted with EtOAc (3×30 mL) and the organic layers were dried and evaporated. The crude product was chromatographed (CHCl₃-MeOH—NH₄OH 20:5:0.5 v/v) to give 20 as an amorphous solid (63% yield); ¹H NMR (CDCl₃) δ 1.67 (br s, 2H), 2.96 (m, 2H), 3.13 (m, 2H), 3.88 (s, 3H), 3.98 (s, 2H), 6.47 (d, 1H, J=7.6 Hz), 6.89 (d, 1H, J=8.1 Hz), 7.01 (t, 1H, J=7.9 Hz), 7.75 (br s, 1H). Anal. (C₁₂H₁₄N₂O) C, H, N.

N-4-(1,2,3,4-Tetrahydro-5-methoxy-β-carbolin-2-yl)butyl]isoquinoline-3-carboxamide (1d). To a suspension of 1,2,3,4-tetrahydro-5-methoxy-β-carboline 20 (94.0 mg, 0.55 mmol) and K₂CO₃ (218.6 mg, 1.57 mol) in dry acetonitrile (10.0 mL) bromo-derivative 6b (137.0 mg, 0.45 mmol) and a catalytic amount of NaI were added and the resulting mixture was heated at reflux for 18 h. Then the mixture was filtered and the filtrate was evaporated to dryness under reduced pressure. The residue was suspended in water (10.0 mL) and extracted with Et₂O (2×25 mL) and dichloromethane (1×25 mL). The combined organic layers was evaporated under reduced pressure and the crude product was purified by means of flash chromatography (0.5% methanol in chloroform) affording to 1d as yellow oil (30% yield); ¹H NMR (CDCl₃) δ 1.74 (m, 4H), 2.64 (m, 2H), 2.82 (m, 2H), 3.03 (m, 2H), 3.57 (m, 4H), 3.86 (s, 3H), 6.44 (d, 1H, J=7.5 Hz), 6.92 (m, 2H), 7.72 (m, 2H), 7.97 (m, 2H), 8.39 (br s, 1H), 8.59 (s, 1H), 9.07 (s, 1H); ES-MS m/z 429 (100) [M+H]⁺, 256, 227. Anal. (C₂₆H₂₈N₄O₂) C, H, N.

### N-[4-(3,4-Dihydro-6-methoxypyrazino[1,2-a]indol-2(1H)-yl)butyl]isoquinoline-3-carboxamide (Compound 1-5/1e)

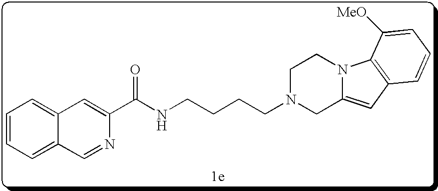

N-[4-(3,4-Dihydro-6-methoxypyrazino[1,2-a]indol-2(1H)-yl)butyl]isoquinoline-3-carboxamide (1e). The title compound was prepared using the bromo-derivative 6b (98.0 mg, 0.32 mmol) and the amino-derivative 11 (0.32 mmol) and following the procedure described to obtain 1b. The compound 1e was obtained as colourless oil (55% yield); ¹H NMR (CDCl₃) δ 1.75 (m, 4H), 2.60 (m, 2H), 2.89 (t, 2H, J=5.5 Hz), 3.57 (q, 2H, J=6.1 Hz), 3.79 (s, 2H), 3.86 (s, 3H), 4.49 (t, 2H, J=5.6 Hz), 6.12 (s, 1H), 6.53 (d, 1H, J=7.6 Hz), 6.93 (t, 1H, J=7.7 Hz), 7.10 (d, 1H, J=7.9 Hz), 7.70 (m, 2H), 7.97 (m, 2H), 8.39 (br s, 1H), 8.59 (s, 1H), 9.05 (s, 1H); ES-MS m/z 879 [2M+Na]⁺, 451 [M+Na]⁺, 429 (100) [M+H]⁺. Anal. (C₂₆H₂₈N₄O₂) C, H, N.

### 3-[5-[4-(3-Chlorophenyl)piperazin-1-yl]pentyloxy]isoquinoline (Compound 1-6/1 f)

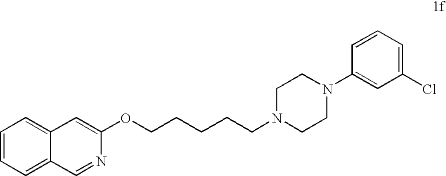

3-(5-Bromopentyloxy)isoquinoline (22). To a solution of isoquinolin-3-ol 21 (200.0 mg, 1.37 mmol) in dry DMF (5.0 mL) 1,5-dibromopentane (204.0 μL, 1.50 mmol) was added and the mixture was stirred at room temperature for 10 min. Then caesium carbonate (538.0 mg, 1.64 mmol) was added and the mixture was heated at 65° C. for 12 h. After cooling at room temperature methyl-tert-butyl-ether (MTBE) (20.0 mL) and water (15.0 mL) were added and the mixture was extracted with MTBE (3×25 mL). The collected organic layers were dried over Na₂SO₄, filtered and evaporated. The residue was chromatographed (dichloromethane) to afford 197.0 mg of pure 22 as yellow oil (49% yield). ¹H NMR (CDCl₃) δ 1.64 (m, 2H), 1.89 (m, 4H), 3.43 (t, 2H, J=6.5 Hz), 4.34 (t, 2H, J=6.3 Hz), 6.97 (s, 1H), 7.33 (m, 1H), 7.54 (t, 1H, J=7.2 Hz), 7.66 (d, 1H, J=8.3 Hz), 7.85 (d, 1H, J=8.2 Hz), 8.92 (s, 1H); ES-MS m/z 296 (100) [M+H]⁺, 146. Anal. (C₁₄H₁₆BrNO) C, H, N.

3-[5-[4-(3-Chlorophenyl)piperazin-1-yl]pentyloxy]isoquinoline (1f). To a stirred solution of 22 (430.0 mg, 1.46 mmol) in dry acetonitrile (30.0 mL) under argon, 1-(3-chlorophenyl)piperazine hydrochloride (338.7 mg, 1.46 mmol) and triethylamine (329.0 μL, 2.36 mmol) were added and the solution was refluxed for 4 h under stirring. The solvent was removed under reduced pressure, water was added and the mixture was extracted with dichloromethane (3×10 mL). The organic layers were dried and concentrated and the crude product was chromatographed (50% n-hexane in EtOAc) to give 320.0 mg of 1f (78.2% yield) as white solid: mp (EtOAc) 88-89° C.; ¹H NMR (CDCl₃) δ 1.52-1.76 (m, 4H), 1.88 (q, 2H, J=6.6 Hz), 2.42 (t, 2H, J=7.2 Hz), 2.57 (m, 4H), 3.18 (m, 4H), 4.35 (t, 2H, J=6.4 Hz), 6.73-6.85 (m, 3H), 6.97 (s, 1H), 7.13 (t, 1H, J=8.0 Hz), 7.34 (dd, 1H, J=8.3, 6.4 Hz), 7.54 (dd, 1H, J=7.9, 6.4 Hz), 7.66 (d, 1H, J=8.1 Hz), 7.85 (d, 1H, J=8.2 Hz), 8.93 (s, 1H); ES-MS m/z 410 (100) [M+H]⁺, 265. Anal. (C₂₄H₂₈ClN₃O) C, H, N.

### N-[4-[4-(m-Tolyl)piperazin-1-yl]butyl]benzo[b]furan-2-carboxamide (Compound 1-7/1g)

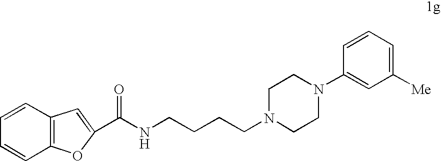

N-[4-[4-(m-Tolyl)piperazin-1-yl]butyl]benzo[b]furan-2-carboxamide (1g). To a stirred solution of 6a (0.62 g, 2.09 mmol) in dry acetonitrile (30.0 mL) under argon, 1-(m-tolyl)piperazine dihydrochloride (0.52 g, 2.09 mmol) and triethylamine (0.62 mL, 4.60 mmol) were added and the solution was refluxed overnight under stirring. The solvent was removed under reduced pressure, water was added and the mixture was extracted with dichloromethane (3×30 mL). The organic layers were dried and concentrated and the crude product was chromatographed (6% methanol in chloroform) to give 0.42 g of 1g (52% yield) as white solid: mp 119-120° C.; ¹H NMR (CDCl₃) δ 1.70 (m, 4H), 2.31 (s, 3H), 2.46 (t, 2H, J=6.6 Hz), 2.62 (t, 4H, J=4.9 Hz), 3.23 (t, 4H, J=4.9 Hz), 3.52 (q, 2H, J=6.1 Hz), 6.70 (m, 3H), 7.00 (br s, 1H), 7.13 (t, 1H, J=4.4 Hz), 7.18-7.48 (m, 4H), 7.66 (d, 1H, J=7.7 Hz); ES-MS m/z 805 (100) [2M+Na]⁺, 414 [M+Na]⁺, 392 [M+H]⁺; ¹³C NMR (CDCl₃) δ 22.0, 24.5, 27.7, 39.4, 49.3, 53.5, 58.1, 110.5, 111.9, 113.4, 117.1, 120.9, 122.9, 123.9, 127.0, 127.9, 129.2, 139.0, 149.2, 151.5, 154.9, 159.1. Anal. (C₂₄H₂₉N₃O₂) C, H, N.

### 4-[4-(2,3-Dichlorophenyl)piperazin-1-yl]butyl 1H-indole-2-carboxylate (Compound 1-8/1h)

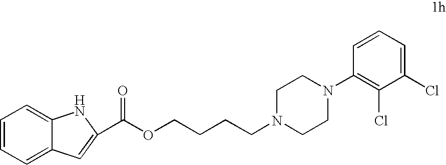

4-Hydroxybutyl 1H-indole-2-carboxylate (5c). To a solution of butane-1,4-diol (0.46 mL, 5.21 mmol), dimethylaminopyridine (DMAP) (68.41 mg, 0.56 mmol), and the 1H-indole-2-carboxylic acid 4c (1.0 g, 6.20 mmol) in dry dichloromethane (20.0 mL) at 0° C. was added dropwise over 45 min a solution of DCC (1.4 g, 6.76 mmol) in dry dichloromethane (10.0 mL). The resulting suspension was stirred for an additional 1 h at 0° C., the cooling bath was removed, and the mixture was stirred for an additional 12 h. The mixture was filtered through Celite® and evaporated to dryness under reduced pressure. The product was purified by means of flash chromatography (7.5% methanol in chloroform) to give 5c as white amorphous solid (45% yield). ¹H NMR (CDCl₃) δ 1.81 (m, 4H), 2.08 (br s, 1H), 3.73 (t, 2H, J=6.0 Hz), 4.39 (t, 2H, J=6.2 Hz), 7.14 (t, 1H, J=7.4 Hz), 7.27 (m, 3H), 7.42 (d, 1H, J=8.2 Hz), 7.68 (d, 1H, J=8.0 Hz), 9.39 (br s, 1H); ES-MS m/z 232 (100) [M−H]⁻ 160, 116. Anal. (C₁₃H₁₅NO₃) C, H, N.

4-Bromobutyl 1H-indole-2-carboxylate (6c). The title compound was prepared starting from 5c (0.45 g, 1.93 mmol) and following the procedure described to obtain 6a. The compound 6c was obtained as white solid (91% yield): mp (ethyl acetate) 85-85° C.; ¹H NMR (CDCl₃) δ δ 1.93 (m, 4H), 3.44 (t, 2H, J=6.2 Hz), 4.37 (t, 2H, J=6.0 Hz), 7.15 (t, 1H, J=7.3 Hz), 7.30 (m, 2H), 7.45 (d, 1H, J=8.2 Hz), 7.69 (d, 1H, J=8.0 Hz), 9.81 (br s, 1H). Anal. (C₁₃H₁₄BrNO₂) C, H, N.

4-[4-(2,3-Dichlorophenyl)piperazin-1-yl]butyl 1H-indole-2-carboxylate (1h). To a stirred solution of 6c (220.0 mg, 0.74 mmol) in dry acetonitrile (20.0 mL) under argon, 1-(2,3-dichloro)piperazine hydrochloride (198.0 mg, 0.74 mmol) and triethylamine (167.0 μL, 1.20 mmol) were added and the solution was refluxed overnight under stirring. The solvent was removed under reduced pressure, water was added and the mixture was extracted with dichloromethane (3×30 mL). The organic layers were dried and concentrated and the crude product was chromatographed (5% methanol in chloroform) to give 1h (60% yield) as white solid: mp (methanol) 136-137° C.; ¹H NMR (CDCl₃) δ 1.79 (m, 4H), 2.50 (t, 2H, J=6.7 Hz), 2.66 (m, 4H), 3.07 (m, 4H), 4.40 (t, 2H, J=5.9 Hz), 6.93 (m, 1H), 7.08-7.35 (m, 5H), 7.43 (d, 1H, J=8.1 Hz), 7.69 (d, 1H, J=7.9 Hz), 9.16 (br s, 1H); ES-MS m/z 468 [H+Na]⁺, 446 (100) [M+Na]⁺. Anal. (C₂₃H₂₅Cl₂N₃O₂) C, H, N.

### 2-[4-[4-(3-Chlorophenyl)piperazin-1-yl]butyl]-3,4-dihydropyrazino[1,2-a]indol-1(2H)-one (Compound 1-9/1i)

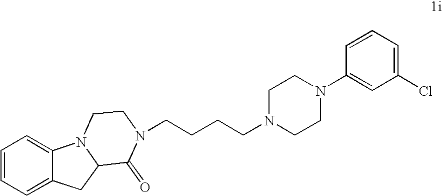

2-[4-[4-(3-Chlorophenyl)piperazin-1-yl]butyl]-3,4-dihydropyrazino[1,2-a]indol-1(2H)-one (1i). The title compound was prepared starting from 15 (410.0 mg, 1.28 mmol) and following the procedure described to obtain 1c. The compound 1i was obtained as white solid (68% yield): mp (EtOAc) 180-181° C.; ¹H NMR (CDCl₃) δ 1.64 (m, 4H), 2.43 (t, 2H, J=7.1 Hz), 2.56 (t, 4H, J=4.9 Hz), 3.17 (t, 4H, J=4.9 Hz), 3.62 (t, 2H, J=6.8 Hz), 3.75 (t, 2H, J=5.9 Hz), 4.22 (m, 3H), 6.76 (m, 3H), 6.84 (d, 1H, J=2.0 Hz), 7.14 (m, 2H), 7.30 (m, 2H), 7.69 (d, 1H, J=7.9 Hz); ES-MS m/z 437 [M+H]⁺; ¹³C NMR (CDCl₃) δ 24.0, 25.5, 40.2, 46.0, 46.2, 48.5, 53.0, 58.0, 106.0, 109.4, 113.7, 115.6, 119.1, 120.6, 122.6, 124.3, 127.5, 129.4, 129.9, 134.9, 136.3, 152.3, 159.8. Anal. (C₂₅H₂₉ClN₄O) C, H, N.

### N-[4-[4-(Pyridin-2-yl)piperazin-1-yl]butyl]isoquinoline-3-carboxamide (Compound 1-10/1j)

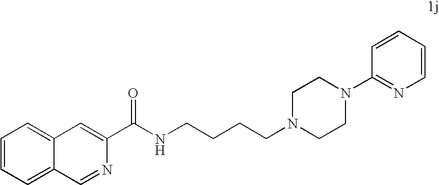

N-[4-[4-(Pyridin-2-yl)piperazin-1-yl]butyl]isoquinoline-3-carboxamide (1j). To a stirred solution of 6b (190.0 mg, 0.62 mmol) in dry acetonitrile (20.0 mL) under argon, 1-(2-pyridin-2-yl)piperazine hydrochloride (101.0 mg, 0.62 mmol) and triethylamine (141.0 μL, 1.0 mmol) were added and the solution was refluxed overnight under stirring. The solvent was removed and the crude product was chromatographed (10% methanol in chloroform) to give 225.0 mg of 1j (93.4% yield) as white solid: mp (methanol) 108-109° C.; ¹H NMR (CDCl₃) δ 1.62 (m, 4H), 2.38 (m, 2H), 2.49 (m, 4H), 3.52 (m, 6H), 6.54 (m, 2H), 7.40 (m, 1H), 7.65 (m, 2H), 7.91 (t, 2H, J=8.6 Hz), 8.11 (d, 1H, J=4.7 Hz), 8.32 (br s, 1H), 8.54 (s, 1H), 9.06 (s, 1H); ES-MS m/z 412 (100) [M+Na⁺], 390 [M+H⁺], 242. Anal. (C₂₃H₂₇N₅O) C, H, N.

### 2-[4-(4-m-Tolyl)piperazin-1-yl]butyl]-3,4-dihydropyrazino[1,2-a]indol-1(2H)-one (Compound 1-11/1k)

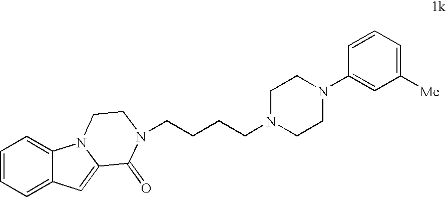

2-[4-(4-m-Tolyl)piperazin-1-yl]butyl]-3,4-dihydropyrazino[1,2-a]indol-1(2H)-one (1k). Starting from 15 (127.0 mg, 0.40 mmol) and 1-(m-tolyl)piperazine dihydrochloride (0.40 mmol), the title compound was prepared following the procedure described for 1g. The product 1k was obtained as white solid (40.0% yield): mp (methanol) 155-156° C.; ¹H NMR (CDCl₃) δ 1.68 (m, 4H), 2.36 (s, 3H), 2.49 (m, 2H), 2.60 (t, 4H, J=5.0 Hz), 3.18 (t, 4H, J=4.9 Hz), 3.66 (m, 2H), 3.78 (t, 2H, J=3.0 Hz), 4.25 (m, 2H), 6.70 (m, 4H), 7.14 (m, 2H), 7.31 (m, 2H), 7.70 (d, 1H, J=8.2 Hz); ES-MS m/z 416 [M+H]⁺. Anal. (C₂₆H₃₂N₄O) C, H, N.

### 3-[5-(4-m-Tolylpiperazin-1-yl)pentyloxy]isoquinoline (Compound 1-12/1l)

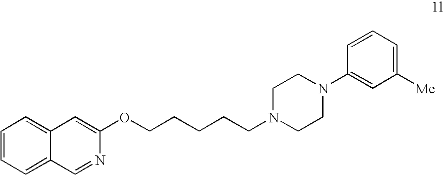

3-[5-(4-m-Tolylpiperazin-1-yl)pentyloxy]isoquinoline (1l). Starting from 22 (101.0 mg, 0.34 mmol) and 1-(m-tolyl)piperazine dihydrochloride (0.34 mmol), the title compound was prepared following the procedure described for 1f. The product 1l was obtained as white solid (65.0% yield): mp (methanol) 94-95° C.; ¹H NMR (CDCl₃) δ 1.72 (m, 4H), 1.92 (m, 2H), 2.30 (s, 3H), 2.42 (t, 2H, J=6.8 Hz), 2.60 (m, 4H), 3.19 (m, 4H), 4.34 (t, 2H, J=6.5 Hz), 6.70 (m, 3H), 6.97 (s, 1H), 7.13 (t, 1H, J=8.1 Hz), 7.55 (t, 1H, J=7.2 Hz), 7.67 (d, 1H, J=8.1 Hz), 7.86 (d, 1H, J=8.2 Hz), 8.93 (s, 1H). Anal. (C₂₅H₃₁N₃O) C, H, N.

### N-[4-[4-(m-Tolyl)piperazin-1-yl]butyl]isoquinoline-3-carboxamide (Compound 1-13/1m)

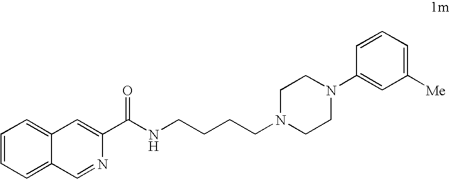

N-[4-[4-(m-Tolyl)piperazin-1-yl]butyl]isoquinoline-3-carboxamide (1 m). Starting from 6b (1.0 g, 3.26 mmol) and 1-(m-tolyl)piperazine dihydrochloride (3.26 mmol), the title compound was prepared following the procedure described for 1g. The product 1m was obtained as yellowish solid (48.0% yield): mp (methanol) 152-153° C.; ¹H NMR (CDCl₃) δ 1.68 (m, 4H), 2.30 (s, 3H), 2.46 (t, 2H, J=6.8 Hz), 2.60 (t, 4H, J=5.0 Hz), 3.20 (t, 4H, J=4.9 Hz), 3.58 (q, 2H, J=6.4 Hz), 6.70 (m, 3H), 7.14 (t, 1H, J=8.1 Hz), 7.72 (m, 2H), 8.00 (t, 2H, J=8.0 Hz), 8.33 (br s, 1H), 8.61 (s, 1H), 9.14 (s, 1H); ES-MS m/z 403 [M+H]⁺; ¹³C NMR (DMSO-_{d6}) 21.3, 22.0, 27.1, 39.1, 46.2, 51.1, 55.7, 114.1, 117.5, 121.1, 121.9, 128.7, 129.0, 129.7, 130.2, 133.0, 136.5, 139.0, 143.0, 150.0, 151.7, 164.1, 170.0. Anal. (C₂₅H₃₀N₄O) C, H, N.

### 7-[4-[4-(2,3-Dichlorophenyl)piperazin-1-yl]butoxy]pyrrolo[1,2-a]quinoxalin-4(5H)-one (Compound 1-14/1n)

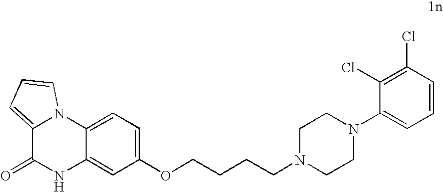

7-Hydroxypyrrolo[1,2-a]quinoxalin-4(5H)-one (24). A solution of 23 (100.0 mg, 0.35 mmol) in THF (70.0 mL) was hydrogenated at atmospheric pressure over 10% Pd—C (1.62 mg) for 16 h. The catalyst was removed by filtration, the solvent was evaporated, and the residue was purified by means of flash chromatography (15% methanol in chloroform) to afford compound 24 in quantitative yield as an amorphous solid. ¹H NMR (DMSO-_{d6}) δ 6.57 (m, 2H), 6.69 (m, 1H), 6.90 (m, 1H), 7.80 (d, 1H, J=8.8 Hz), 7.98 (s, 1H), 9.62 (s, 1H), 11.03 (s, 1H). Anal. (C₁₁H₈N₂O₂) C, H, N.

7-(4-Bromobutoxy)pyrrolo[1,2-a]quinoxalin-4(5H)-one (25). To a solution of compound 24 (50.0 mg, 0.25 mmol) in dry DMF (5.0 mL) 1,4-dibromobutane (64.0 μL, 0.29 mmol) was added and the mixture was stirred at room temperature for 10 min. Then caesium carbonate (98.0 mg, 0.30 mmol) was added and the mixture was heated at 65° C. for 12 h. After cooling at room temperature methyl-tert-butylether (MTBE) (10.0 mL) and water (5.0 mL) were added and the mixture was extracted with MTBE (3×25 mL). The collected organic layers were dried over Na₂SO₄, filtered and evaporated. The residue was chromatographed (15% n-hexane in EtOAc) to afford pure 25 as yellow oil (22.5% yield). ¹H NMR (CD₃OD) δ 1.64 (m, 2H), 1.96 (m, 4H), 3.52 (m, 2H), 3.99 (m, 2H), 6.62 (m, 1H), 6.77 (m, 2H), 7.10 (d, 1H, J=3.5 Hz), 7.73 (m, 1H), 7.83 (d, 1H, J=1.5 Hz). Anal. (C₁₅H₁₅BrN₂O₂) C, H, N.

7-[4-[4-(2,3-Dichlorophenyl)piperazin-1-yl]butoxy]pyrrolo[1,2-a]quinoxalin-4(5H)-one (1n). Starting from 25 (100.0 mg, 0.50 mmol) and (2,3-dichloro)phenylpiperazine dihydrochloride (0.50 mmol), the title compound was prepared following the procedure described for 1f. The product 1n was obtained as white amorphous solid (81.0% yield). ¹H NMR (DMSO-_{d6}) δ 1.67 (m, 4H), 2.44 (m, 6H), 2.95 (m, 4H), 3.99 (t, 2H, J=5.9 Hz), 6.77 (m, 2H), 6.94 (m, 1H), 7.08 (m, 1H), 7.25 (m, 2H), 7.92 (d, 1H, J=8.6 Hz), 8.04 (s, 1H), 11.03 (s, 1H). Anal. (C₂₅H₂₆Cl₂N₄O₂) C, H, N.

### N-[4-[4-(3-Methoxyphenyl)piperazin-1-yl]butyl]isoquinoline-3-carboxamide (Compound 1-15/1 o)

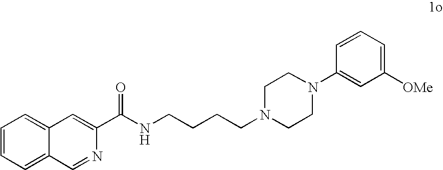

N-[4-[4-(3-Methoxyphenyl)piperazin-1-yl]butyl]isoquinoline-3-carboxamide (1o). Starting from 6b (1.0 g, 2.39 mmol) and (3-methoxy)phenylpiperazine (458.9 mg, 2.39 mmol), the title compound was prepared following the procedure described for 1g. The product 1o was obtained as a colourless oil (54.0% yield). ¹H NMR (CDCl₃) δ 1.66 (m, 4H), 2.44 (t, 2H, J=6.8 Hz), 2.59 (t, 4H, J=4.9 Hz), 3.19 (t, 4H, J=4.9 Hz), 3.56 (q, 2H, J=6.3 Hz), 3.77 (s, 3H), 6.47 (m, 3H), 7.14 (t, 1H, J=8.1 Hz), 7.73 (m, 2H), 7.99 (t, 2H, J=8.2 Hz), 8.32 (br s, 1H), 8.60 (s, 1H), 9.12 (s, 1H); ES-MS m/z 441 [M+Na]⁺, 419 [M+H]⁺. Anal. (C₂₅H₃₀N₄O₂) C, H, N.

### N-[4-[4-(3-Trifluoromethyl phenyl)piperazin-1-yl]butyl]indole-2-carboxamide (Compound 1-16/1p)

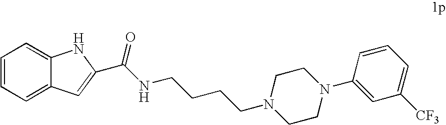

N-[1-(4-Hydroxy)butyl]indole-2-carboxamide (5d). To a solution of 2-indolecarboxylic acid 4c (150.0 mg, 0.93 mmol) in dry dichloromethane (20.0 mL), 1-hydroxybenzotriazole hydrate (460.0 mg, 1.03 mmol) and 1,3-dicyclohexylcarbodiimide (210.0 mg, 1.03 mmol) were added at 0° C. under argon; the suspension was warmed to room temperature and stirred for 1 h. Then 4-amino-1-butanol (93.6 μL, 1.03 mmol) was added and the mixture was stirred at room temperature for 16 h. The resulting suspension was filtered through Celite, washed with chloroform, and the filtrate evaporated. The crude product was purified by flash chromatography (10% methanol in chloroform) to give of 5d as colorless as colorless prisms (93% yield): mp (methanol) 108-109° C.; ¹H NMR (CDCl₃) δ1.67 (m, 4H), 3.52 (q, 2H, J) 11.5, 5.6) 3.72 (t, 2H, J) 5.8 Hz), 6.65 (br s, 1H), 6.82 (s, 1H), 7.11 (d, 1H, J) 8.0 Hz), 7.29 (m, 1H), 7.39 (d, 1H, J) 7.7 Hz), 7.59 (d, 1H, J) 7.8 Hz), 9.25 (br s, 1H). Anal. (C₁₃H₁₆N₂O₂) C, H, N.

N-[1-(4-Bromo)butyl]indole-2-carboxamide (6d). To a stirred solution of 5d (170.0 mg, 0.73 mmol) in acetonitrile (25.0 mL), triphenylphosphine (0.86 g, 3.22 mmol) and carbon tetrabromide (1.06 g, 3.22 mmol) were added at room temperature. After 2 h, the mixture was quenched with 15% NaOH and extracted with ethyl acetate. The organic layers were dried and evaporated. The residue was purified by means of flash chromatography (20% n-hexane in ethyl acetate) to afford 6b as a colorless prisms (84% yield): mp (ethyl acetate) 133-134° C.; ¹H NMR (CDCl₃) δ 1.96 (m, 4H), 3.56 (m, 4H), 7.28 (m, 5H), 7.60 (d, 1H, J) 7.6 Hz), 9.80 (br s, 1H). Anal. (C₁₃H₁₅BrN₂O) C, H, N.

N-[4-[4-(3-Trifluoromethylphenyl)piperazin-1-yl]butyl]indole-2-carboxamide (1p). Starting from 6d (35.0 mg, 0.12 mmol) and (3-trifluoromethyl)phenylpiperazine (27.3 mg, 0.12 mmol) the title compound was prepared following the procedure described for 1g and was obtained as a yellow oil (56.0% yield). ¹H NMR (CDCl₃) δ 1.82 (m, 4H), 2.43 (m, 2H), 2.57 (m, 4H), 3.21 (m, 4H), 3.54 (m, 2H), 6.58 (m, 1H), 6.83 (s, 1H), 7.11 (m, 3H), 7.31 (m, 2H), 7.45 (d, 1H, J=8.0 Hz), 7.61 (d, 1H, J=7.9 Hz), 10.08 (br s, 1H); ES-MS m/z 445 [M+H]⁺. Anal. (C₂₄H₂₇F₃N₄O) C, H, N.

### N-[2-(1H-Indol-3-yl)ethyl]-3-(4-m-tolylpiperazin-1-yl)propanamide (Compound 1-17/1q)

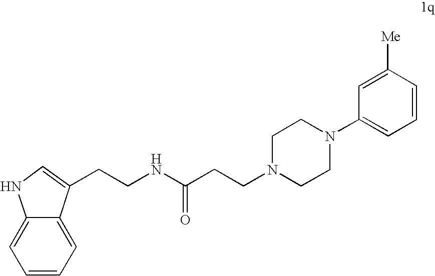

N-[2-(1H-Indol-3-yl)ethyl]-3-bromopropanamide (27). To a solution of tryptamine (1.0 g, 6.24 mmol) in dry dichloromethane (10.0 mL), 3-bromopropanol chloride (691.0 μl, 6.86 mmol) and triethylamine (870.0 μl, 6.24 mmol) were added and the solution was stirred into a microwave oven for 1 min at 200 W. Then the solvent was evaporated, water was added to the residue ed was extracted with EtOAc (3×20 mL). The crude product was purified by means of flash chromatography (10% methanol in chloroform) to afford 27 as white solid (22.0% yield): mp (EtOAc)=106-107° C. ¹H NMR (CDCl₃) δ 2.64 (t, 2H, J=6.4 Hz), 2.98 (t, 2H, J=6.6 Hz), 3.56 (m, 4H), 5.61 (br s, 1H), 7.06 (m, 1H), 7.18 (m, 2H), 7.36 (d, 1H, J=7.9 Hz), 7.60 (d, 1H, J=7.6 Hz), 8.10 (br s, 1H); ES-MS m/z 319 [M+Na]⁺, 295 [M+H]⁺. Anal. Anal. (C₁₃H₁₅BrN₂O) C, H, N.

N-[2-(1H-Indol-3-yl)ethyl]-3-(4-m-tolylpiperazin-1-yl)propanamide (1q). To a stirred solution of 27 (200.0 mg, 0.68 mmol) in dry acetonitrile (20.0 mL) under argon, 1-(m-tolyl)piperazine dihydrochloride (0.68 mmol) and triethylamine (141.0 μL, 1.0 mmol) were added; the solution was refluxed overnight while stirring. The solvent was removed under reduced pressure, water was added and the mixture was extracted with dichloromethane (3×10 mL). The organic layers were dried and concentrated and the crude product was chromatographed (10% methanol in chloroform) to give 1q (50% yield) as white amorphous solid. ¹H NMR (CDCl₃) δ 2.23-2.41 (m, 9H), 2.51 (m, 2H), 2.74 (m, 4H), 2.96 (t, 2H, J=6.5 Hz), 3.64 (q, 2H, J=6.3 Hz), 6.60 (m, 2H), 6.69 (m, 1H), 6.98-7.28 (m, 5H), 7.58 (m, 1H), 8.09 (br s, 1H), 8.20 (br s, 1H); ES-MS m/z 413 [M+Na]⁺, 391 [M+H]⁺. Anal. (C₂₄H₃₀N₄O) C, H, N.

### N-[2-(1H-Indol-3-yl)ethyl]-3-[4-(3-methoxyphenyl)piperazin-1-yl]propanamide (Compound 1-18/1r)

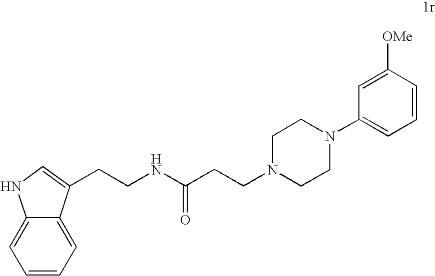

N-[2-(1H-Indol-3-yl)ethyl]-3-[4-(3-methoxyphenyl)piperazin-1-yl]propanamide (1r). Starting from 27 (100.0 mg, 0.34 mmol) and (3-methoxy)phenylpiperazine (0.34 mmol) the title compound was prepared following the procedure described for 1q and was obtained as a white amorphous solid (47.0% yield). ¹H NMR (CDCl₃) δ 2.33 (m, 6H), 2.46 (m, 2H), 2.73 (m, 4H), 2.95 (m, 2H), 3.62 (m, 2H), 3.79 (s, 3H), 6.38 (m, 3H), 6.97 (s, 1H), 7.03-7.27 (m, 4H), 7.57 (d, 1H, J=7.4 Hz), 8.24 (br s, 1H), 8.48 (br s, 1H). Anal. (C₂₄H₃₀N₄O₂) C, H, N.

### (S)-(−)-N-[4-[4-(m-Tolyl)piperazin-1-yl]butyl]-1,2,3,4-tetrahydroisoquinoline-2-carboxamide (Compound 1-19/1s)

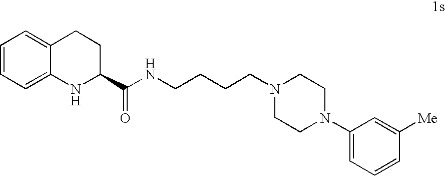

2-Methyl(S)-(−)-1-(benzyloxycarbonyl)-1,2,3,4-tetrahydroquinoline-2-carboxylate ((S)-30). To a solution of amino ester (S)-28 (160.0 mg, 0.84 mmol) in aq. NaHCO₃ (2M), benzyl chloroformate (158.2 mg, 0.92 mmol) was added dropwise in 30 min. The mixture was stirred for 1.5 h at room temperature then evaporated. The residue was extracted with EtOAc (3×20 mL) and the organic layers were dried and evaporated. The crude product was purified was purified by means of flash chromatography (20% acetone in n-hexane) to give compound (S)-30 as colourless oil (80% yield). [α]²⁰_{D}=−50.0° (c=0.94, MeOH); ¹H NMR (CDCl₃) δ 1.81 (m, 1H), 2.31-2.43 (m, 1H), 2.43-2.69 (m, 2H), 3.61 (s, 3H), 4.96 (t, 1H, J=7.6 Hz), 5.24 (s, 2H), 6.97-7.08 (m, 2H), 7.16-7.23 (m, 1H), 7.24-7.35 (m, 5H), 7.81 (d, 1H, J=7.4 Hz); ESI-MS m/z 325 [M⁺], 281, 266, 222, 190, 130, 91. Anal. (C₁₉H₁₉NO₄) C, H, N.

(S)-(−)-1-(Benzyloxycarbonyl)-1,2,3,4-tetrahydroquinoline-2-carboxylic acid [(S)-31]. To a solution of (S)-30 (218.5 mg, 0.67 mmol) in methanol and water (3:2) NaOH (27.0 mg, 0.67 mmol) was added and the mixture was heated to reflux for 2 h. Then the solvents were evaporated, water was added to the residue and the mixture was acidified with HCl 1N. The aqueous layer was extracted with chloroform. (3×15 mL) and the collected organic layers were dried and evaporated. The crude product was purified by means of flash chromatography (CHCl₃/MeOH/CH₃COOH 9:1:0.1) to afford (S)-31 as amorphous solid and in quantitative yield. ¹H NMR (CDCl₃) δ 1.81-1.99 (m, 1H), 2.31-2.43 (m, 1H), 2.46-2.69 (m, 2H), 3.61 (s, 3H), 4.96 (t, 1H, J=7.6 Hz), 5.24 (s, 2H), 6.97-7.08 (m, 2H), 7.16-7.23 (m, 1H), 7.24-7.35 (m, 5H), 7.81 (d, 1H, J=7.4 Hz); ESI-MS m/z 310 [M]⁺ (100), 266, 202. Anal. (C₁₈H₁₇NO₄) C, H, N. [α]²⁰_{D}−50° (c=0.98, MeOH).

(S)-(−)-N-[4-(1-Hydroxy)butyl]-1-(benzyloxycarbonyl)-1,2,3,4-tetrahydroisoquinoline-2-carboxamide [(S)-32]. To a solution acid (S)-31 (980.5 mg, 3.15 mmol) in dry dichloromethane (20.0 mL), 1-hydroxybenzotriazole hydrate (HOBT) (920.0 mg, 6.80 mmol) and 1,3-dicyclohexylcarbodiimide (1.40 g, 6.80 mmol) were added at 0° C. under argon; the suspension was warmed to room temperature and stirred for 1 h. Then 4-amino-1-butanol (0.56 mL, 6.16 mmol) was added and the mixture was stirred overnight at room temperature. The resulting suspension was filtered through Celite®, washed with chloroform (3×25 mL) and the filtrate evaporated. The crude product was purified by means of flash chromatography (10% methanol in chloroform) to give (S)-32 as colourless oil (84% yield). ¹H NMR (CDCl₃) δ 1.17-1.40 (m, 4H), 2.03-2.32 (m, 2H), 2.50-2.74 (m, 2H), 3.11 (m, 2H), 3.36-3.43 (m, 2H), 4.92 (t, 1H, J=6.8 Hz), 5.12-5.27 (m, 2H), 6.43 (br s, 1H), 6.96-7.17 (m, 3H), 7.31 (m, 5H), 7.63 (d, 1H, J=8.10 Hz); ESI-MS m/z 405 [M+Na]⁺ (100); MS/MS (405) m/z 361, 270. [α]²⁰_{D}+41.9° (c=1.56, CHCl₃). Anal. (C₂₂H₂₆N₂O₄) C, H, N.

(S)-(−)-N-[4-(1-Bromo)butyl]-1-(benzyloxycarbonyl)-1,2,3,4-tetrahydroisoquinoline-2-carboxamide [(S)-33]. To a vigorous stirred solution of (S)-32 (1.0 g, 2.62 mmol) in dry acetonitrile (50.0 mL), triphenylphosphine (0.86 g, 3.22 mmol) and carbon tetrabromide (1.06 g, 3.22 mmol) were added at room temperature. After 2 h the mixture was quenched with 15% NaOH and the heterogeneous mixture was extracted with ethyl acetate (EtOAc) (3×25 mL). The organic layers were dried and evaporated. The residue was chromatographed (20% n-hexane in ethyl acetate) to afford 0.58 g (91% yield) of (S)-33 as yellow oil (33% yield). ¹H NMR (CDCl₃) δ 1.34-1.61 (m, 4H), 2.15-2.26 (m, 2H), 2.57-2.78 (m, 2H), 3.02-3.30 (m, 4H), 5.00 (t, 1H, J=6.70 Hz), 5.14-5.30 (m, 2H), 6.07 (br s, 1H), 6.99-7.21 (m, 3H), 7.33 (m, 5H), 7.61 (d, 1H, J=8.02 Hz); ESI-MS m/z 467 [M+Na]⁺. [α]²⁰_{D}−50.9° (c=0.53, CHCl₃). Anal. (C₂₂H₂₅BrN₂O₃) C, H, N.

(S)-(−)-N-[4-[4-(m-Tolyl)piperazin-1-yl]butyl]-2-(benzyloxycarbonyl)-1,2,3,4-tetrahydroisoquinoline-2-carboxamide [(S)-34a]. To a stirred solution of (S)-33 (180.4 mg, 0.40 mmol) in dry acetonitrile (10.0 mL) under argon, 1-(m-tolyl)piperazine dihydrochloride (150.9 mg, 0.40 mmol) and triethylamine (62.0 μL, 0.46 mmol) were added and the solution was refluxed overnight under stirring. The solvent was removed under reduced pressure, water was added and the mixture was extracted with dichloromethane (3×30 mL). The organic layers were dried and concentrated and the crude product was chromatographed (6% methanol in chloroform) to give (S)-34a as yellow oil (40% yield). ¹H NMR (CDCl₃) δ 1.25-1.37 (m, 4H), 2.15-2.33 (m, 7H), 2.47-2.57 (m, 4H), 2.62-2.79 (m, 2H), 3.12-3.27 (m, 6H), 4.97 (t, 1H, J=6.65 Hz), 5.16-5.31 (m, 2H), 6.12 (br s, 1H), 6.65-6.73 (m, 3H), 7.03-7.24 (m, 3H), 7.34 (m, 5H), 7.64 (d, 1H, J=8.10 Hz). [α]²⁰_{D}=−34.3° (c=1.75, CHCl₃). Anal. (C₃₃H₄₀N₄O₃) C, H, N.

(S)-(−)-N-[4-[4-(m-Tolyl)piperazin-1-yl]butyl]-1,2,3,4-tetrahydroisoquinoline-2-carboxamide (1s). To a solution of (S)-34a (50.0 mg, 0.15 mmol) in methanol and EtOAc (1:1) catalytic Pd on carbon 5% was added under argon and the suspension was hydrogenated at 60 psi for 8 h. The mixture was then filtered through Celite® and the filtrate was evaporated. The crude product was chromatographed (10% methanol in chloroform) to afford 1s as colourless oil (90% yield). ¹H NMR (CDCl₃) δ 1.20-1.58 (m, 4H), 1.86-1.95 (m, 4H), 2.19-1.37 (m, 3H), 2.39-2.46 (m, 2H), 2.57-2.77 (m, 4H), 3.22-3.33 (m, 6H), 3.64-3.84 (m, 1H), 6.63-6.73 (m, 3H), 6.96-7.03 (m, 2H), 7.07-7.18 (m,); ESI-MS m/z 407 [M]⁺ (100); MS/MS (407) m/z 300, 276, 258, 248, 231, 189, 177, 161, 132. [α]²⁰_{D}=−42.1° (c=1.26, MeOH). Anal. (C₂₅H₃₄N₄O) C, H, N.

### (R)-(+)-N-[4-[4-(m-Tolyl)piperazin-1-yl]butyl]-1,2,3,4-tetrahydroisoquinoline-2-carboxamide (Compound 1-20/1t)

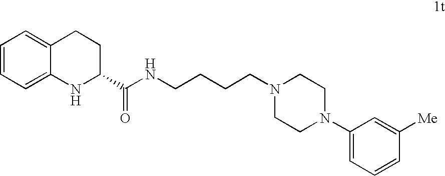

(R)-(+)-1-(Benzyloxycarbonyl)-1,2,3,4-tetrahydroquinoline-2-carboxylic acid [(R)-31]. The title compound was prepared starting from (R)-29 (1.30 g, 7.34 mmol) and following the procedure described to obtain (S)-31. The compound (R)-29 was obtained as colourless oil (81% yield). ¹H NMR (CDCl₃) δ 1.85-1.99 (m, 1H), 2.35-2.50 (m, 1H), 2.58-2.79 (m, 2H), 4.99 (t, 1H, J=7.74 Hz), 5.19-5.34 (m, 2H), 6.99-7.10 (m, 2H), 7.18-7.25 (m, 1H), 7.32-7.38 (m, 5H), 7.78 (d, 1H, J=7.92 Hz), 9.94 (br s, 1H). [α]²⁰_{D}+44.6° (c=0.74, MeOH). Anal. (C₁₈H₁₇NO₄) C, H, N

(R)-(+)-N-[4-(1-Hydroxy)butyl]-1-(benzyloxycarbonyl)-1,2,3,4-tetrahydroisoquinoline-2-carboxamide [(R)-32]. The title compound was prepared starting from (R)-31 (418.2 mg, 1.34 mmol) and following the procedure described to obtain (S)-32. The compound (R)-32 was obtained as colourless oil (72% yield). ¹H NMR (CDCl₃) δ1.21-1.40 (m, 4H), 2.01-2.10 (m, 1H), 2.14-2.28 (m, 1H), 2.56-2.74 (m, 2H), 2.82 (br s, 1H), 3.10-3.13 (m, 2H), 3.40-3.45 (m, 2H), 4.87-4.94 (m, 1H), 5.11-5.26 (m, 2H), 6.51 (br s, 1H), 6.95-7.17 (m, 3H), 7.30 (m, 5H), 7.64 (d, 1H, J=8.14 Hz); ESI-MS m/z 405 [M+Na]⁺ (100), 267. [α]²⁰_{D}+50° (c=1.94, CHCl₃). Anal. (C₂₂H₂₆N₂O₄) C, H, N.

(R)-(+)-N-[4-(1-Bromo)butyl]-1 (benzyloxycarbonyl)-1,2,3,4-tetrahydroisoquinoline-2-carboxamide [(R)-33]. The title compound was prepared starting from (R)-32 (368.9 mg, 0.97 mmol) and following the procedure described to obtain (S)-33. The compound (R)-33 was obtained as colourless oil (33% yield). ¹H NMR (CDCl₃) δ 1.40-1.64 (m, 4H), 2.20-2.31 (m, 2H), 2.62-2.80 (m, 2H), 3.07-3.28 (m, 4H), 4.99 (t, 1H, J=6.67 Hz), 5.16-5.31 (m, 2H), 6.07 (br s, 1H), 7.04-7.21 (m, 3H), 7.34 (m, 5H), 7.62 (d, 1H, J=8.06 Hz); [α]²⁰_{D}+50.9° (c=0.15, CHCl₃). Anal. (C₂₂H₂₅BrN₂O₃) C, H, N.

(R)-(+)-N-[4-[4-(m-Tolyl)piperazin-1-yl]butyl]-2-(benzyloxycarbonyl)-1,2,3,4-tetrahydroisoquinoline-2-carboxamide [(R)-34a]. The title compound was prepared starting from (R)-33 (70.0 mg, 0.16 mmol) and (m-tolyl)piperazine dihydrochloride (39 mg, 0.16 mmol) following the procedure described to obtain (S)-34a. The compound (R)-34a was obtained as colourless oil (40% yield). ¹H NMR (CDCl₃) δ 1.25-1.46 (m, 4H), 2.16-2.28 (m, 4H), 2.31 (s, 3H), 2.48-2.58 (m, 4H), 2.64-2.80 (m, 2H), 3.12-3.25 (m, 6H), 4.95-5.01 (m, 1H), 5.16-5.32 (m, 2H), 6.05 (br s, 1H), 6.66-6.73 (m, 3H), 7.04-7.21 (m, 3H), 7.25 (s, 1H), 7.35 (m, 5H), 7.63 (d, 1H, J=8.11 Hz). [α]²⁰_{D}=+34.3° (c=0.19, CHCl₃). Anal. (C₃₃H₄₀N₄O₃) C, H, N.

(R)-(+)-N-[4-[4-(m-Tolyl)piperazin-1-yl]butyl]-1,2,3,4-tetrahydroisoquinoline-2-carboxamide (1t). The title compound was prepared starting from (R)-34a (50.0 mg, 0.15 mmol) following the procedure described to obtain 1s. Compound 1t was obtained as colourless oil (85% yield). ¹H NMR (CDCl₃) δ 1.20-1.58 (m, 4H), 1.86-1.95 (m, 4H), 2.19-1.37 (m, 3H), 2.39-2.46 (m, 2H), 2.57-2.77 (m, 4H), 3.22-3.33 (m, 6H), 3.64-3.84 (m, 1H), 6.63-6.73 (m, 3H), 6.96-7.03 (m, 2H), 7.07-7.18 (m,); ESI-MS m/z 407 [M⁺] (100); MS/MS (407) m/z 300, 276, 258, 248, 231, 189, 177, 161, 132. [α]²⁰_{D}=+ 42.1° (c=1.26, MeOH). Anal. (C₂₅H₃₄N₄O) C, H, N.

### (R)-(+)-N-[4-(4-Phenylpiperazin-1-yl)butyl]-1,2,3,4-tetrahydroisoquinoline-2-carboxamide (Compound 1-21/1u)

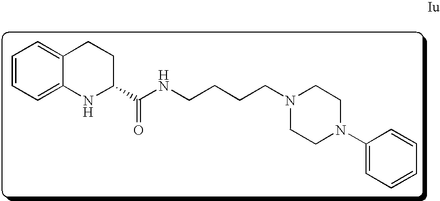

(R)-(+)-N-[4-[4-(3-Chloro)phenylpiperazin-1-yl]butyl]-2-(benzyloxycarbonyl)-1,2,3,4-tetrahydroisoquinoline-2-carboxamide [(R)-34b]. The title compound was prepared starting from (R)-33 (140.0 mg, 0.31 mmol) and (3-chloro)phenylpiperazine hydrochloride (73.4 mg, 0.31 mmol) following the procedure described to obtain (S)-34a. The compound (R)-34b was obtained as colourless oil (40% yield). ¹H NMR (CDCl₃) δ 1.25-1.36 (m, 4H), 2.20-2.45 (m, 4H), 2.48-2.59 (m, 4H), 2.64-2.80 (m, 2H), 3.12-3.21 (m, 6H), 4.98 (t, 1H, J=6.65 Hz), 5.16-5.31 (m, 2H), 6.02 (br s, 1H), 6.74-6.85 (m, 3H), 7.04-7.21 (m, 3H), 7.25 (s, 1H), 7.34 (m, 5H), 7.63 (d, 1H, J=7.97 Hz); ESI-MS m/z 584 [M+Na]⁺, 561 [M+H⁺] (100), 508. Anal (C₃₂H₃₇N₄O₃) C, H, N. [α]²⁰_{D}+31° (c=0.19, CHCl₃). Anal. (C₃₂H₃₇ClN₄O₃) C, H, N.

(R)-(+)-N-[4-(4-Phenylpiperazin-1-yl)butyl]-1,2,3,4-tetrahydroisoquinoline-2-carboxamide (1u). The title compound was prepared starting from (R)-34b (50.0 mg, 0.15 mmol) following the procedure described to obtain 1s. The compound 1u was obtained as colourless oil (92% yield). ¹H NMR (CDCl₃) δ 1.49-1.53 (m, 4H), 1.79-1.95 (m, 2H), 2.24-2.46 (m, 2H), 2.48-2.61 (m, 5H), 2.65-2.78 (m, 1H), 3.13-3.18 (m, 4H), 3.24-3.33 (m, 2H), 3.93-4.00 (m, 1H), 6.59-6.74 (m, 2H), 6.80-7.06 (m, 5H), 7.20-7.28 (m, 2H). ESI-MS m/z 393 [M⁺] (100), 132. Anal (C₂₄H₃₂N₄O) C, H, N. [α]²⁰_{D}=+41.66° (c=0.24, CHCl₃). Anal. (C₂₃H₃₀N₄O) C, H, N.

### N-(4-(4-(Phenylpiperazin-1-yl)butyl)benzo[b]furan-2-carboxamide (Compound 1-22)

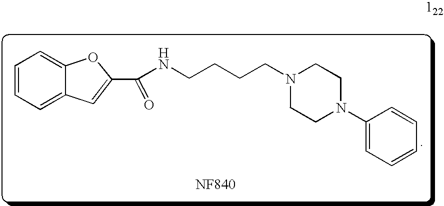

Starting from 7₁ and 1-phenylpiperazine, the title compound was prepared following the procedure described to obtain 1₇. Compound 1₂₂ was obtained as white solid (70% yield): mp (methanol) 149-150° C.; ¹H NMR, 300 MHz, (CDCl₃) δ 1.71 (m, 4H), 2.47 (m, 2H), 2.64 (m, 4H), 3.24 (m, 4H), 3.53 (m, 2H), 6.89 (m, 3H), 7.02 (br s, 1H), 7.28 (m, 3H), 7.39 (m, 1H), 7.46 (m, 2H), 7.66 (m, 1H). ESI-MS m/z 400 [M+Na⁺], 377 [M+H⁺] (100). Anal (C₂₃H₂₇N₃O₂) C, H, N

### N-(4-(4-(2,3-Dimethylphenyl)piperazin-1-yl)butyl)benzo[b]furan-2-carboxamide (Compound 1-23)

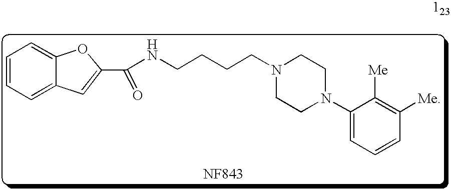

Starting from 7₁ and 4-(2,3-dimethylphenyl)piperazine, the title compound was prepared following the procedure described to obtain 1₇. Compound 1₂₃ was obtained as white solid (73% yield): mp (methanol) 151-152° C. ¹H NMR, 200 MHz, (CDCl₃) δ 1.73 (m, 4H), 2.21 (s, 3H), 2.25 (s, 3H), 2.47 (m, 2H), 2.62 (m, 4H), 2.93 (m, 4H), 3.52 (m, 2H), 6.87 (m, 2H), 7.05 (m, 2H), 7.35 (m, 3H), 7.64 (m, 1H). ESI-MS m/z 428 [M+Na⁺], 406 [M+H⁺] (100). Anal (C₂₅H₃₁N₃O₂) C, H, N.

### N-(4-(4-(3-Methoxyphenyl)piperazin-1-yl)butyl)benzo[b]furan-2-carboxamide (Compound 1-24)

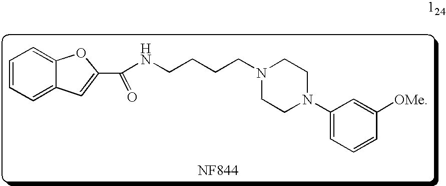

Starting from 7₁ and 4-(3-methoxyphenyl)piperazine, the title compound was prepared following the procedure described to obtain 1₇. Compound 1₂₄ was obtained as white solid (70% yield): mp (methanol) 104-105° C. ¹H NMR, 200 MHz, (CDCl₃) δ1.62 (m, 4H), 2.40 (m, 2H), 2.56 (m, 4H), 3.19 (m, 4H), 3.49 (m, 2H), 3.74 (s, 3H), 6.44 (m, 3H), 7.25 (m, 5H), 7.61 (m, 1H). ESI-MS m/z 430 [M+Na⁺], 408 [M+H⁺] (100). Anal (C₂₄H₂₉N₃O₃) C, H, N.

### Experimental Procedure for Compound 1-25

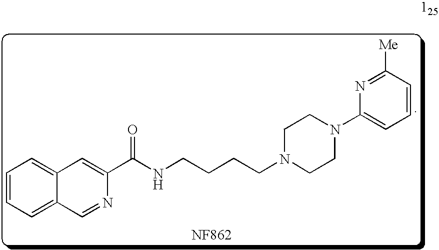

tert-Butyl-4-(6-methylpyridin-2-yl)piperazine-1-carboxylate (3₂). In a sealed tube, 2-bromo-6-methylpyridine (461 mg, 2.68 mmol) Pd₂(dba)₂ (2%), BINAP (4%), and sodium t-butoxide (386.4 mg, 4.02 mmol) were added to N-Boc-piperazine (500 mg, 2.68 mmol) and the solids were dissolved in dry toluene (5 mL). The mixture was stirred at 70° C. for 90 min., filtered over Celite®, washing with etylacetate and the organic layer was evaporated under reduced pressure. The crude was purified by flash chromatography (40% etylacetate in hexane) to give 3₂ as pail yellow solid (95% yield): mp (methanol) 84-85° C.; ¹H NMR, 200 MHz, (CDCl₃) δ 1.41 (s, 9H), 2.31 (s, 3H), 3.42 (m, 8H), 6.37 (m, 2H), 7.28 (m, 1H). ESI-MS m/z 300 [M+Na⁺], 278 [M+H⁺] (100). Anal (C₁₅H₂₃N₃O₂) C, H, N.

1-(6-Methylpyridin-2-yl)piperazine trifluoroacetate (4₁). Trifluoroacetic acid (4 mL) was added to 3₂, cooling in ice bath, and the mixture was stirred for 60 min. at room temperature. The crude was concentrated and washed with diethylether till the solid became colorless.

N-(4-(4-(6-Methylpyridin-2-yl)piperazin-1-yl)butyl)isoquinoline-3-carboxamide (Compound 1-25). Starting from 7₂ and 4₁, the title compound was prepared following the procedure described to obtain 1₃. Compound 1₂₅ was obtained as white solid (70% yield): mp (methanol) 124-125° C. ¹H NMR, 200 MHz, (CDCl₃) δ 1.71 (m, 4H), 2.38 (s, 3H), 2.47 (m, 2H), 2.56 (m, 4H), 3.55 (m, 6H), 6.44 (m, 2H), 7.30 (m, 1H), 7.72 (m, 2H), 8.00 (m, 2H), 8.33 (br s, 1H), 8.60 (m, 1H), 9.14 (m, 1H). ESI-MS m/z 426 [M+Na⁺], 404 [M+H⁺] (100). Anal (C₂₄H₂₉N₅O) C, H, N.

### N-(4-(4-Phenyl piperazin-1-yl)butyl)isoquinoline-3-carboxamide (Compound 1-26)

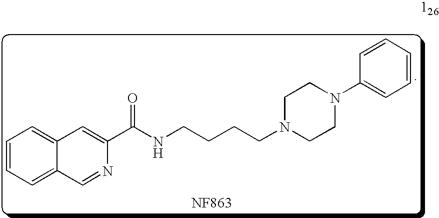

Starting from 7₂ and 1-phenylpiperazine, the title compound was prepared following the procedure described to obtain 1₃. Compound 1₂₆ was obtained as white solid (76% yield): mp (methanol) 153-154° C. ¹H NMR, 300 MHz, (CDCl₃) δ 1.71 (m, 4H), 2.46 (m, 2H), 2.62 (m, 4H), 3.21 (m, 4H), 3.57 (m, 2H), 6.88 (m, 3H), 7.26 (m, 2H), 7.72 (m, 2H), 8.00 (m, 2H), 8.36 (br s, 1H), 8.61 (m, 1H), 9.14 (m, 1H). ESI-MS m/z 411 [M+Na⁺], 389 [M+H⁺] (100). Anal (C₂₄H₂₈N₄O) C, H, N.

### N-(4-(4-(6-Methylpyridin-2-yl)piperazin-1-yl)butyl)benzofuran-2-carboxamide (Compound 1-27)

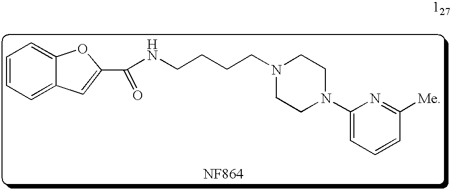

Starting from 7₁ and 4₁, the title compound was prepared following the procedure described to obtain 1₇. Compound 1₂₇ was obtained as white solid (75% yield): mp (methanol) 107-109° C. ¹H NMR, 200 MHz, (CDCl₃) δ 1.70 (m, 4H), 2.39 (s, 3H), 2.45 (m, 2H), 2.58 (m, 4H), 3.54 (m, 6H), 6.45 (m, 2H), 7.02 (br s, 1H), 7.35 (m, 5H), 7.65 (m, 1H). ESI-MS m/z 415 [M+Na⁺], 393 [M+H⁺] (100). Anal (C₂₃H₂₈N₄O₂) C, H, N.

### Experimental Procedure for Compound 1-28

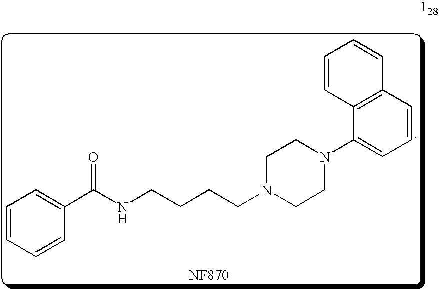

tert-Butyl-4-(aphthalene-1-yl)piperazine-1-carboxylate (3₃). Starting from 1-bromonaphthalene (200 mg, 0.97 mmol) the title compound was prepared following the procedure described to obtain 3₂: ¹H NMR, 200 MHz, (CDCl₃) δ 1.55 (s, 9H), 3.05 (m, 4H), 3.72 (m, 4H), 7.05 (m, 1H), 7.49 (m, 4H), 7.82 (m, 1H), 8.22 (m, 1H). ESI-MS m/z 335 [M+Na⁺], 313 [M+H⁺] (100). Anal (C₁₉H₂₄N₂O₂) C, H, N.

1-(Naphthalen-1-yl)piperazine trifluoroacetate (4₂). Starting from 3₃ the title compound was prepared following the procedure described to obtain 4₁.

N-(4-(4-(Naphthalen-1-yl)piperazin-1-yl)butyl)benzamide (Compound 1-28). Starting from 7₅ and 4₂, the title compound was prepared following the procedure described to obtain 1₃. Compound 1₂₈ was obtained as yellow oil (70% yield): ¹H NMR, 200 MHz, (CDCl₃) δ 1.68 (m, 4H), 2.49 (m, 4H), 2.70 (m, 4H), 3.34 (m, 2H), 7.01 (m, 2H), 7.43 (m, 6H), 7.78 (m, 3H), 8.18 (m, 1H). ESI-MS m/z 410 [M+Na⁺], 388 [M+H⁺] (100). Anal (C₂₅H₂₉N₃O) C, H, N.

### N-(4-(4-Phenylpiperazin-1-yl)butyl)quinoline-2-carboxamide (Compound 1-29)

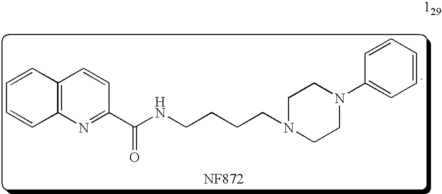

Starting from 7₂ and 1-phenylpiperazine, the title compound was prepared following the procedure described to obtain 1₃. Compound 1₂₉ was obtained as white solid (70% yield): mp (methanol) 120-121° C. ¹H NMR, 200 MHz, (CDCl₃) δ 1.69 (m, 4H), 2.46 (m, 2H), 2.61 (m, 4H), 3.20 (m, 4H), 3.56 (m, 2H), 6.86 (m, 3H), 7.24 (m, 2H), 7.59 (m, 1H), 7.75 (m, 1H), 7.85 (m, 1H), 8.09 (m, 1H), 8.29 (m, 2H). ESI-MS m/z 411 [M+Na⁺], 389 [M+H⁺] (100). Anal (C₂₄H₂₈N₄O) C, H, N.

### N-(4-(4-m-Tolylpiperazin-1-yl)butyl)quinoline-2-carboxamide (Compound 1-30)

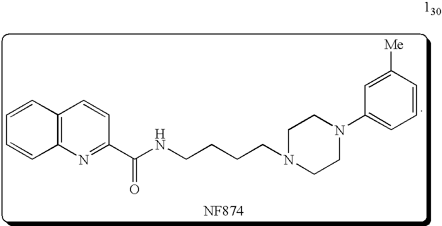

Starting from 7₂ and 4-(3-methylphenyl)piperazine, the title compound was prepared following the procedure described to obtain 1₃. Compound 1₃₀ was obtained as yellow oil: ¹H NMR, 200 MHz, (CDCl₃) δ 1.72 (m, 4H), 2.30 (s, 3H), 2.47 (m, 2H), 2.61 (m, 4H), 3.19 (m, 4H), 3.47 (m, 2H), 6.69 (m, 4H), 7.11 (m, 1H), 7.59 (m, 1H), 7.75 (m, 1H), 7.85 (m, 1H), 8.09 (m, 1H), 8.33 (m, 2H). ESI-MS m/z 403 [M+H⁺] (100). Anal (C₂₅H₃₀N₄O) C, H, N.

### N-(4-(4-(3-Methoxyphenyl)piperazin-1-yl)butyl)-1-methyl-1H-indole-2-carboxamide (Compound 1-31)

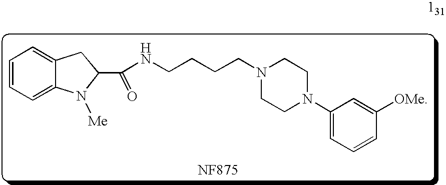

Starting from 7₇ and 4-(3-methoxyphenyl)piperazine, the title compound was prepared following the procedure described to obtain 1₃. Compound 1₃₁ was obtained as white solid (60% yield): mp (methanol) 141-142° C. ¹H NMR, 200 MHz, (CDCl₃) δ 1.69 (m, 4H), 2.48 (m, 2H), 2.65 (m, 4H), 3.08 (m, 4H), 3.47 (m, 2H), 3.84 (s, 3H), 4.03 (s, 3H), 6.90 (m, 5H), 7.16 (m, 1H), 7.32 (m, 2H), 7.60 (m, 1H). ESI-MS m/z 421 [M+H⁺] (100). Anal (C₂₅H₃₂N₄O₂) C, H, N.

### Experimental Procedure for Compound 1-32

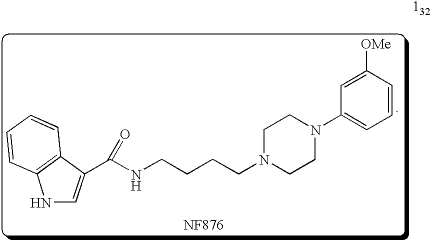

4-(4-(3-Methoxyphenyl)piperazin-1-yl)butanenitrile. (37). To a stirred solution of 1-(3-methoxyphenyl)piperazine (36) (100.0 mg, 0.52 mmol) in acetonitrile (10.0 mL), 4-bromobutanenitrile (84.7 mg, 0.57 mmol) and potassium carbonate (107.6 mg, 0.78 mmol) were added at room temperature. The mixture was heated to reflux over night then filtrated and evaporated. The crude product was purified by means of flash chromatography (10% methanol in chloroform) to give 98.0 mg (73% yield) of 37 as a yellow oil: ¹H NMR, 200 MHz, (CDCl₃) δ 1.83 (m, 2H), 2.49 (m, 8H), 3.17 (m, 4H), 3.77 (s, 3H), 6.48 (m, 3H), 7.16 (m, 1H). ESI-MS m/z 282 [M+H⁺] 260 [M+H⁺] (100). Anal (C₁₅H₂₁N₃O) C, H, N.

4-(4-(3-Methoxyphenyl)piperazin-1-yl)butan-1-amine. (38). To a stirred solution of 37 (300.0 mg, 1.16 mmol) in dry methanol (15.0 mL), at 0° C., nickel(II) chloride hexahydrate (28.0 mg, 0.12 mmol) and sodium borohydride (307.2 mg, 8.12 mmol) were added. The mixture was stirred at room temperature for 90 min, then filtered over Celite®, washing with methanol, and the filtrate was evaporated under reduced pressure. The residue was extracted with EtOAc (3×30 mL), the organic layers were dried and concentrated and the crude product was chromatographed (15% n-hexane in EtOAc) to give 38 (60% yield): ¹H NMR, 200 MHz, (CDCl₃) δ 1.54 (m, 4H), 2.40 (m, 4H), 2.58 (m, 4H), 3.19 (m, 4H), 3.76 (s, 3H), 6.46 (m, 3H), 7.16 (m, 1H). ESI-MS m/z 286 [M+H⁺] 264 [M+H⁺] (100). Anal (C₁₅H₂₅N₃O) C, H, N.

1H-Indole-3-carbonylchloride (40). To a solution of 1H-indole-3-carboxylic acid (39) (100.0 mg, 0.6 mmol) in benzene dry (2.0 mL), thionylchloride (130.0 μL, 1.80 mmol) was added and the mixture was heated to reflux for 120 min. The crude was washed with benzene (2×10 mL) and evaporated to give 40 in quantitative yield. ¹H NMR, 200 MHz, (CDCl₃) δ 10.85 (br s, 1H), 8.20 (m, 1H), 7.48 (m, 1H), 7.30 (m, 1H), 7.19 (m, 1H), 7.03 (m, 1H). Anal (C₉H₆ClNO) C, H, N.

N-(4-(4-(3-Methoxyphenyl)piperazin-1-yl)butyl)-1H-indole-3-carboxamide (Compound 1-32). To a stirred solution of 40 (100.0 mg, 0.6 mmol) and 37 (158.0 mg, 0.6 mmol) in dry dichloromethane (15.0 mL), pyridine (145 μL, 1.8 mmol) was added. The mixture was stirred at room temperature overnight. Sodium bicarbonate saturated solution was added and the mixture, extracted with EtOAc (3×15 mL), was dried and evaporated. The crude product was purified by means of flash chromatography (10% methanol in chloroform) to give 1₃₂ (50% yield) as a white solid: mp (methanol) 154-155° C. ¹H NMR, 200 MHz, (CDCl₃) δ 1.65 (m, 4H), 2.41 (m, 2H), 2.56 (m, 4H), 3.13 (m, 4H), 3.50 (m, 2H), 3.77 (s, 3H), 6.45 (m, 2H), 7.19 (m, 3H), 7.38 (m, 1H), 7.66 (m, 1H), 7.95 (m, 1H), 9.79 (br s, 1H). ESI-MS m/z 429 [M+Na⁺], 407 [M+H⁺] (100). Anal (C₂₄H₃₀N₄O₂) C, H, N.

### N-(4-(4-Benzylpiperazin-1-yl)butyl)benzofuran-2-carboxamide (Compound 1-33)

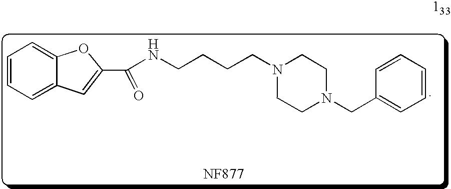

Starting from 7₁ and 1-benzylpiperazine, the title compound was prepared following the procedure described to obtain 1₇. Compound 1₃₃ was obtained as yellow oil (70% yield): ¹H NMR, 200 MHz, (CDCl₃) δ 1.64 (m, 4H), 2.39 (m, 2H), 2.50 (m, 8H), 3.49 (m, 4H), 7.04 (br s, 1H), 7.36 (m, 10H), 7.65 (m, 1H). ESI-MS m/z 414 [M+Na⁺], 392 [M+H⁺] (100). Anal (C₂₄H₂₉N₃O₂) C, H, N.

### 7-(5-(4-Phenylpiperazin-1-yl)pentyloxy)pyrrolo[1,2-a]quinoxalin-4(5H)-one (Compound 1-34)

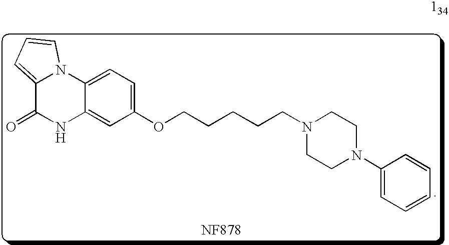

Starting from 26₂ and 1-phenylpiperazine, the title compound was prepared following the procedure described to obtain 1₁₄. Compound 1₃₄ was obtained as amorphous solid (60% yield). ¹H NMR (CDCl₃) δ 1.56 (m, 4H), 1.86 (m, 2H), 2.45 (m, 2H), 2.64 (m, 4H), 3.22 (m, 4H), 4.03 (m, 2H), 6.66 (m, 1H), 6.79 (m, 3H), 6.91 (m, 2H), 7.25 (m, 3H), 7.56 (m, 2H), 10.19 (br s, 1H). ESI-MS m/z 453 [M+Na⁺], 431 [M+H⁺] (100). Anal. (C₂₆H₃₀N₄O₂) C, H, N.

### (S)-1,2,3,4-Tetrahydro-N-(4-(4-phenylpiperazin-1-yl)butyl)quinoline-2-carboxamide (Compound 1-35)

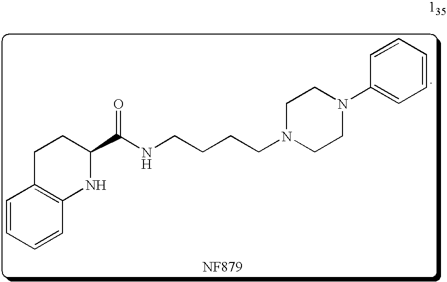

Starting from (S)-34 and 1-phenylpiperazine, the title compound was prepared following the procedure described to obtain 1₂₁. Compound 1₃₅ was obtained as an oil (70% yield): ¹H NMR (CDCl₃) δ 1.25-1.36 (m, 4H), 2.20-2.45 (m, 4H), 2.48-2.59 (m, 4H), 2.64-2.80 (m, 2H), 3.12-3.21 (m, 6H), 4.98 (t, 1H, J=6.65 Hz), 5.16-5.31 (m, 2H), 6.02 (br s, 1H), 6.74-6.85 (m, 3H), 7.04-7.21 (m, 3H), 7.25 (s, 1H), 7.34 (m, 5H), 7.63 (d, 1H, J=7.97 Hz); ESI-MS m/z 584 [M+Na⁺], 561 [M+H⁺] (100). [α]²⁰_{D}−31° (c=0.18, CHCl₃). Anal. (C₂₃H₃₂N₄O) C, H, N.

### Experimental Procedure for Compound 1-36

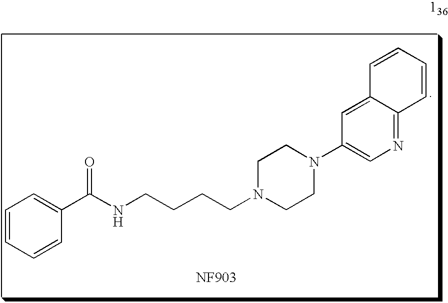

tert-Butyl-4-(quinolin-3-yl)piperazine-1-carboxylate (3₄). Starting from 3-bromoquinoline, the title compound was prepared following the procedure described to obtain 4₁. Compound 3₄ was obtained as white solid: mp (methanol) 114-115° C. ¹H NMR, 200 MHz, (CDCl₃) δ 1.46 (s, 9H), 3.18 (m, 4H), 7.28 (m, 1H), 7.43 (m, 2H), 7.63 (m, 1H), 7.96 (m, 1H), 8.74 (m, 1H). ESI-MS m/z 336 [M+Na⁺] (100). Anal. (C₁₈H₂₃N₃O₂) C, H, N.

3-(4-Methylpiperazin-1-yl)quinoline trifluoroacetate (4₃). Starting from 3₄, the title compound was prepared following the procedure described to obtain (4₁)

N-(4-(4-(Quinolin-3-yl)piperazin-1-yl)butyl)benzamide (Compound 1-36). Starting from 7₅ and 4₃, the title compound was prepared following the procedure described to obtain 1₃. Compound 1₃₆ was obtained as white solid (70% yield): mp (methanol) 124-125° C. ¹H NMR, 200 MHz, (CDCl₃) δ 1.68 (m, 4H), 2.45 (m, 2H), 2.65 (m, 4H), 3.27 (m, 4H), 3.53 (m, 2H), 6.63 (br s, 1H), 7.38 (m, 6H), 7.65 (m, 1H), 7.75 (m, 2H), 7.97 (m, 1H), 8.76 (m, 1H). ESI-MS m/z 389 [M+H⁺] (100). Anal (C₂₄H₂₈N₄O) C, H, N.

### N-(4-(4-m-Tolylpiperazin-1-yl)butyl)picolinamide (Compound 1-37)

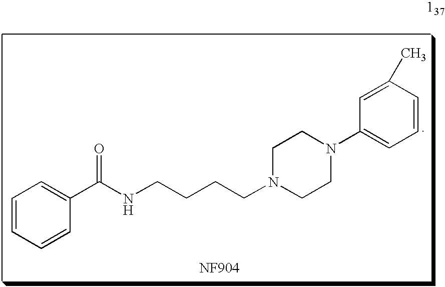

Starting from 7₈ and 4-(3-methylphenyl)piperazine, the title compound was prepared following the procedure described to obtain 1₃. Compound 1₃₇ was obtained as yellow oil (70% yield): ¹H NMR, 200 MHz, (CDCl₃) δ 1.67 (m, 4H), 2.30 (s, 3H), 2.45 (m, 2H), 2.61 (m, 4H), 3.20 (m, 4H), 3.51 (m, 2H), 6.70 (m, 3H), 7.14 (m, 1H), 7.40 (m, 1H), 7.84 (m, 1H), 8.19 (m, 2H), 8.52 (m, 1H). ESI-MS m/z 375 [M+Na⁺], 353 [M+H⁺] (100). Anal (C₂₁H₂₈N₄O) C, H, N.

### 7-(4-(4-Phenylpiperazin-1-yl)butoxy)pyrrolo[1,2-a]quinoxalin-4(5H)-one (Compound 1-38)

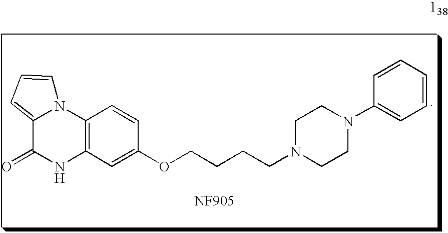

Starting from 26₁, and 1-phenylpiperazine, the title compound was prepared following the procedure described to obtain 1₁₄. Compound 1₃₈ was obtained as amorphous solid (60% yield). ¹H NMR (CDCl₃) δ 1.81 (m, 4H), 2.49 (m, 2H), 2.65 (m, 4H), 3.21 (m, 4H), 4.06 (m, 2H), 6.66 (m, 1H), 6.74 (m, 3H), 6.89 (m, 2H), 7.23 (m, 3H), 7.56 (m, 2H), 10.27 (br s, 1H). ESI-MS m/z 440 [M+Na⁺], 417 [M+H⁺] (100). Anal. (C₂₅H₂₈N₄O₂) C, H, N.

### N-(4-(4-(Quinolin-3-yl)piperazin-1-yl)butyl)isoquinoline-3-carboxamide (Compound 1-39)

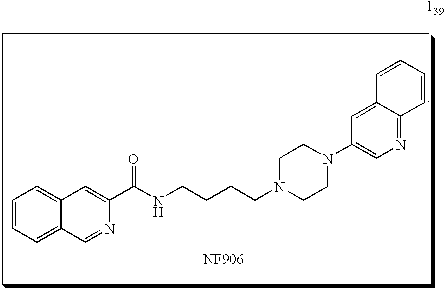

Starting from 7₂ and 4₃, the title compound was prepared following the procedure described to obtain 1₃. Compound 1₃₉ was obtained as white solid (70% yield): mp (methanol) 153-154° C. ¹H NMR, 200 MHz, (CDCl₃) δ 1.80 (m, 4H), 2.37 (m, 2H), 2.67 (m, 4H), 3.32 (m, 4H), 3.75 (m, 2H), 7.30 (m, 1H), 7.45 (m, 2H), 7.67 (m, 4H), 7.93 (m, 2H), 8.33 (br s, 1H), 8.60 (m, 1H), 8.78 (m, 1H), 9.12 (m, 1H). ESI-MS m/z 462 [M+Na⁺], 440 [M+H⁺] (100). Anal (C₂₇H₂₉N₅O) C, H, N.

### N-(4-(4-(3-Methoxyphenyl)piperazin-1-yl)butyl)-6-methylpyridine-2-carboxamide (Compound 1-40)

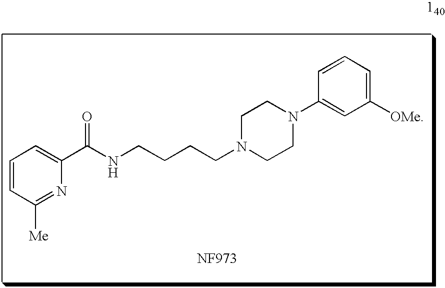

Starting from 7₉ and 4-(3-methoxyphenyl)piperazine, the title compound was prepared following the procedure described to obtain 1₃. Compound 1₄₀ was obtained as yellow oil (78% yield): ¹H NMR, 200 MHz, (CDCl₃) δ 1.64 (m, 4H), 2.41 (m, 2H), 2.53 (s, 3H), 2.59 (m, 4H), 3.17 (m, 4H), 3.49 (m, 2H), 3.75 (s, 3H), 6.45 (m, 3H), 7.17 (m, 2H), 7.67 (m, 1H), 7.97 (m, 1H), 8.14 (br s, 1H). ESI-MS m/z 405 [M+Na⁺], 383 [M+H⁺] (100). Anal (C₂₂H₃₀N₄O₂) C, H, N.

### N-(4-(4-(3-Methoxyphenyl)piperazin-1-yl)butyl)quinoline-3-carboxamide (Compound 1-41)

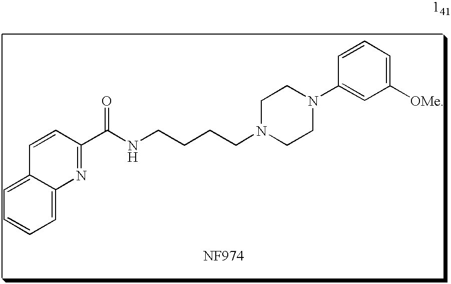

Starting from 7₆ and 4-(3-methoxyphenyl)piperazine, the title compound was prepared following the procedure described to obtain 1₃. Compound 1₄₁ was obtained as yellow oil (78% yield): ¹H NMR, 200 MHz, (CDCl₃) δ 1.63 (m, 4H), 2.41 (m, 2H), 2.56 (m, 4H), 3.15 (m, 4H), 3.54 (m, 2H), 3.73 (s, 3H), 6.43 (m, 4H), 7.13 (m, 1H), 7.55 (m, 1H), 7.70 (m, 1H), 7.81 (m, 1H), 8.06 (m, 1H), 8.26 (m, 1H), 8.31 (br s, 1H). ESI-MS m/z 441 [M+Na⁺], 419 [M+H⁺] (100). Anal (C₂₅H₃₀N₄O₂) C, H, N.

### N-(4-(4-(Pyridin-2-yl)piperazin-1-yl)butyl)quinoline-3-carboxamide (Compound 1-42)

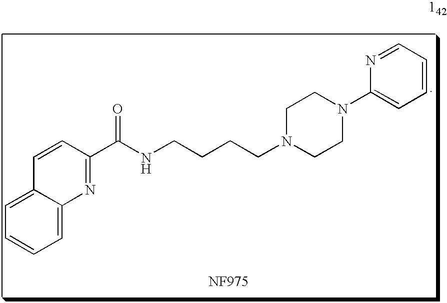

Starting from 7₆ and 1-(pyridin-2-yl)piperazine, the title compound was prepared following the procedure described to obtain 1₃. Compound 1₄₂ was obtained as yellow oil (70% yield): ¹H NMR, 200 MHz, (CDCl₃) δ 1.82 (m, 4H), 2.39 (m, 2H), 2.50 (m, 4H), 3.52 (m, 6H), 6.55 (m, 2H), 7.40 (m, 1H), 7.53 (m, 1H), 7.69 (m, 1H), 7.80 (m, 1H), 8.17 (m, 5H). ESI-MS m/z 412 [M+Na⁺], 390 [M+H⁺] (100). Anal (C₂₃H₂₇N₅O) C, H, N.

### N-(4-(4-Phenylpiperazin-1-yl)butyl)picolinamide (Compound 1-43)

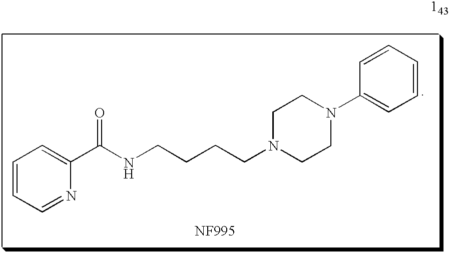

Starting from 7₈ and 1-phenylpiperazine, the title compound was prepared following the procedure described to obtain 1₃. Compound 1₄₃ was obtained as yellow oil (82% yield): ¹H NMR, 400 MHz, (CDCl₃) δ 1.63 (m, 4H), 2.39 (m, 2H), 2.56 (m, 4H), 3.17 (m, 4H), 3.47 (m, 2H), 6.80 (m, 1H), 6.88 (m, 2H), 7.21 (m, 2H), 7.35 (m, 1H), 7.78 (m, 1H), 8.16 (m, 1H), 8.48 (m, 1H). ESI-MS m/z 361 [M+Na⁺], 339 [M+H⁺] (100). Anal (C₂₀H₂₆N₄O) C, H, N.

### N-(4-(4-(3-Methoxyphenyl)piperazin-1-yl)butyl)picolinamide (Compound 1-44)

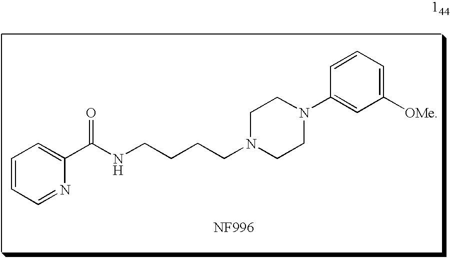

Starting from 7₈ and 4-(3-methoxyphenyl)piperazine, the title compound was prepared following the procedure described to obtain 1₃. Compound 1₄₄ was obtained as yellow oil (72% yield): ¹H NMR, 400 MHz, (CDCl₃) δ 1.57 (m, 4H), 2.33 (m, 2H), 2.49 (m, 4H), 3.11 (m, 4H), 3.43 (m, 2H), 3.68 (s, 3H), 6.32 (m, 2H), 6.38 (m, 1H), 6.45 (m, 1H), 7.07 (m, 1H), 7.30 (m, 1H), 7.72 (m, 1H), 8.11 (m, 1H), 8.43 (m, 1H). ESI-MS m/z 369 [M+H⁺] (100). Anal (C₂₁H₂₈N₄O₂) C, H, N.

### N-(4-(4-(3-Methoxyphenyl)piperazin-1-yl)butyl)benzamide (Compound 1-45)

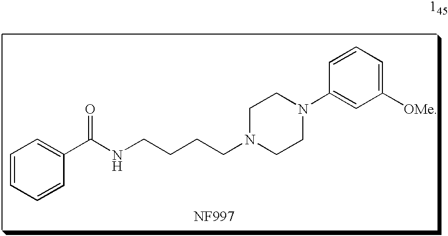

Starting from 7₅ and 4-(3-methoxyphenyl)piperazine, the title compound was prepared following the procedure described to obtain 1₃. Compound 1₄₅ was obtained as white solid (79% yield): mp (methanol) 111-112° C.: ¹H NMR, 400 MHz, (CDCl₃) δ1.62 (m, 4H), 2.39 (m, 2H), 2.54 (m, 4H), 3.13 (m, 4H), 3.42 (m, 2H), 3.75 (s, 3H), 6.40 (m, 2H), 6.49 (m, 1H), 6.83 (br s, 1H), 7.14 (m, 1H), 7.36 (m, 1H), 7.44 (m, 1H), 7.74 (m, 2H). ESI-MS m/z 390 [M+Na⁺], 368 [M+H⁺] (100). Anal (C₂₂H₂₉N₃O₂) C, H, N.

### N-(4-(4-m-Tolylpiperazin-1-yl)butyl)benzamide (Compound 1-46)

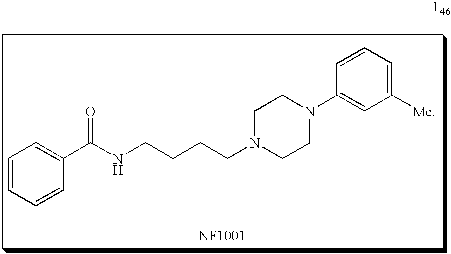

Starting from 7₅ and 4-(3-methylphenyl)piperazine, the title compound was prepared following the procedure described to obtain 1₃. Compound 1₄₆ was obtained as white solid (77% yield): mp (methanol) 126-127° C. ¹H NMR, 400 MHz, (CDCl₃) δ 1.64 (m, 4H), 2.30 (s, 3H), 2.40 (m, 2H), 2.55 (m, 4H), 3.13 (m, 4H), 3.45 (m, 2H), 6.68 (m, 3H), 6.90 (br s, 1H), 7.13 (m, 1H), 7.37 (m, 2H), 7.43 (m, 1H), 7.75 (m, 2H). ESI-MS m/z 374 [M+Na⁺], 352 [M+H⁺] (100). Anal (C₂₂H₂₉N₃O) C, H, N.

### N-(4-(4-Phenylpiperazin-1-yl)butyl)nicotinamide (Compound 1-47)

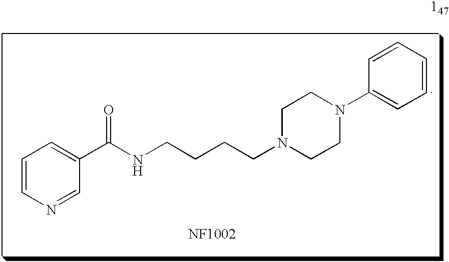

Starting from 7₁₀ and 1-phenylpiperazine, the title compound was prepared following the procedure described to obtain 1₃. Compound 1₄₇ was obtained as white solid (80% yield): mp (methanol) 119-120° C. ¹H NMR, 400 MHz, (CDCl₃) δ 1.65 (m, 4H), 2.42 (m, 2H), 2.56 (m, 4H), 3.12 (m, 4H), 3.46 (m, 2H), 6.84 (m, 3H), 7.11 (br s, 1H), 7.23 (m, 2H), 7.31 (m, 1H), 8.07 (m, 1H), 8.65 (m, 1H), 8.94 (m, 1H). ESI-MS m/z 339 [M+H⁺] (100). Anal (C₂₀H₂₆N₄O) C, H, N.

### N-(4-(4-(6-Methylpyridin-2-yl)piperazin-1-yl)butyl)benzamide (Compound 1-48)

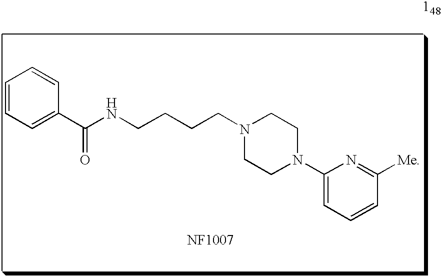

Starting from 7₅ and 4₁, the title compound was prepared following the procedure described to obtain 1₃. Compound 1₄₈ was obtained as white solid (82% yield): mp (methanol) 102-103° C. ¹H NMR, 200 MHz, (CDCl₃) δ 1.59 (m, 4H), 2.34 (m, 5H), 2.46 (m, 4H), 3.46 (m, 6H), 6.39 (m, 2H), 7.07 (br s, 1H), 7.33 (m, 4H), 7.73 (m, 2H). ESI-MS m/z 375 [M+Na⁺], 353 [M+H⁺] (100). Anal (C₂₁H₂₈N₄O) C, H, N.

### Experimental Procedure for Compound 1-49

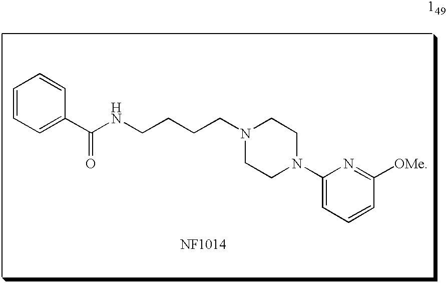

tert-Butyl-4-(6-methoxypyridin-2-yl)piperazine-1-carboxylate (3₅). Starting from 2-bromo-6-methoxypyridine the title compound was prepared following the procedure described to obtain 3₂: ¹H NMR, 200 MHz, (CDCl₃) δ 1.42 (s, 9H), 3.19 (m, 4H), 3.73 (s, 3H), 3.79 (m, 4H), 5.70 (m, 1H), 5.90 (m, 1H), 7.44 (m, 1H), ESI-MS m/z 316 [M+Na⁺], 294 [M+H⁺] (100). Anal (C₁₅H₂₃N₃O₃) C, H, N.

1-(6-Methoxypyridin-2-yl)piperazine (44). Starting from 3₅, the title compound was prepared following the procedure described to obtain 4₁

N-(4-(4-(6-Methoxypyridin-2-yl)piperazin-1-yl)butyl)benzamide (Compound 1-49). Starting from 7₅ and 4₄, the title compound was prepared following the procedure described to obtain 1₃. Compound 1₄₉ was obtained as white solid (85% yield): mp (methanol) 120-121° C. ¹H NMR, 200 MHz, (CDCl₃) δ 1.61 (m, 4H), 2.37 (m, 2H), 2.47 (m, 4H), 3.43 (m, 6H), 3.81 (s, 3H), 6.06 (m, 2H), 6.85 (br s, 1H), 7.38 (m, 4H), 7.72 (m, 2H). ESI-MS m/z 391 [M+Na⁺], 369 [M+H⁺] (100). Anal (C₂₁H₂₈N₄O₂) C, H, N.

### N-(4-(4-(6-Methoxypyridin-2-yl)piperazin-1-yl)butyl)picolinamide (Compound 1-50)

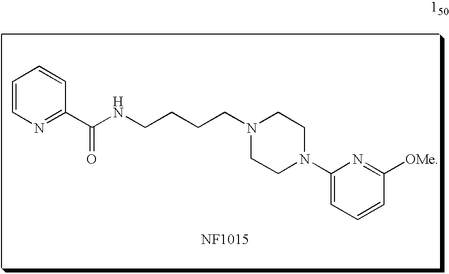

Starting from 7₈ and 4₄, the title compound was prepared following the procedure described to obtain 1₃. Compound 1₅₀ was obtained as yellow oil (80% yield): ¹H NMR, 300 MHz, (CDCl₃) δ 1.63 (m, 4H), 2.37 (m, 2H), 2.48 (m, 4H), 3.47 (m, 4H), 3.80 (m, 3H), 6.05 (m, 2H), 7.34 (m, 2H), 7.77 (m, 1H), 8.14 (m, 2H), 8.47 (m, 1H). ESI-MS m/z 429 [M+Na⁺], 407 [M+H⁺] (100). ESI-MS m/z 376 [M+Na⁺], 354 [M+H⁺] (100). Anal (C₂₀H₂₇N₅O₂) C, H, N.

### N-(4-(4-(6-Methylpyridin-2-yl)piperazin-1-yl)butyl)picolinamide (Compound 1-51)

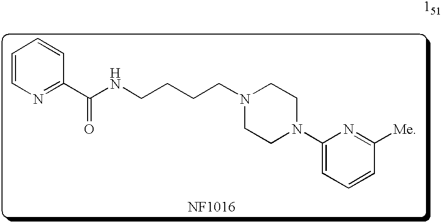

Starting from 7₈ and 4₁, the title compound was prepared following the procedure described to obtain 1₃. Compound 1₅₁ was obtained as yellow (90% yield): ¹H NMR, 300 MHz, (CDCl₃) δ 1.58 (m, 4H), 2.31 (m, 5H), 2.45 (m, 4H), 3.43 (m, 6H), 6.36 (m, 2H), 7.29 (m, 2H), 7.72 (m, 1H), 8.11 (m, 2H), 8.43 (m, 1H). ESI-MS m/z 376 [M+Na⁺], 354 [M+H⁺] (100). Anal (C₂₀H₂₇N₅O) C, H, N.

### Experimental Procedure for Compound 1-52

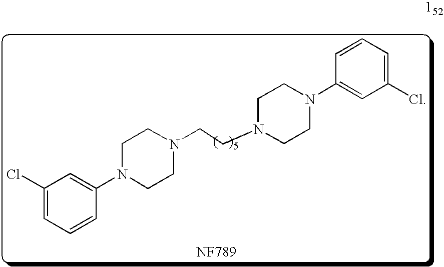

1,6-Bis(4-(3-chlorophenyl)piperazin-1-yl)hexane (Compound 1-52). To a solution of 3-chlorophenylpiperazine (406) (100.0 mg, 0.51 mmol) in acetonitrile dry (15.0 mL), 1,6-dibromohexane (34.72 μL, 0.25 mmol) and TEA (71.1 μL, 0.51 mmol) was added and the mixture was stirred at room temperature overnight. The crude was extracted with dichloromethane (3×10 mL), dried and evaporated. The residue was chromatographed (10% methanol in chloroform) to give 1₅₂ (90% yield) as yellow: ¹H NMR, 200 MHz, (CDCl₃) δ 1.37 (m, 4H), 1.52 (m, 4H), 2.37 (m, 4H), 2.57 (m, 8H), 3.20 (m, 8H), 6.77 (m, 4H), 6.86 (m, 2H), 7.14 (m, 2H). ESI-MS m/z 475 [M+H⁺] (100). Anal (C₂₆H₃₆Cl₂N₄) C, H, N.

### 1,6-Bis(4-(3-methoxyphenyl)piperazin-1-yl)hexane (Compound 1-53)

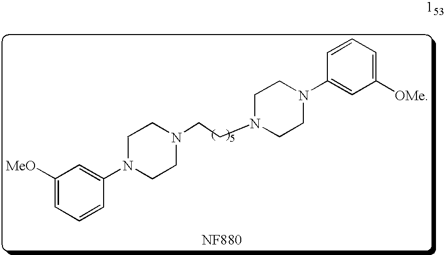

Starting from 4-(3-methoxyphenyl)piperazine, the title compound was prepared following the procedure described to obtain 1₅₂. Compound 1₅₃ was obtained as white solid (85% yield): mp (methanol) 109-110° C. ¹H NMR, 200 MHz, (CDCl₃) δ 1.36 (m, 4H), 1.54 (m, 4H), 2.38 (m, 4H), 2.58 (m, 8H), 3.20 (m, 8H), 3.77 (m, 8H), 6.44 (m, 6H), 7.15 (m, 2H), ESI-MS m/z 467 [M+H⁺] (100). Anal (C₂₈H₄₂N₄O₂) C, H, N.

### 1,6-Bis(4-phenylpiperazin-1-yl)hexane (Compound 1-54)

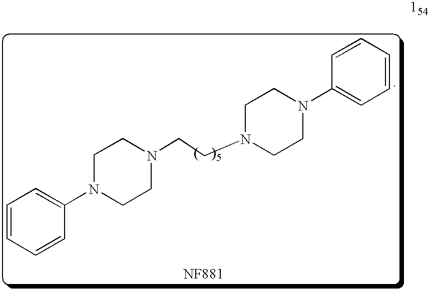

Starting from 1-phenylpiperazine the title compound was prepared following the procedure described to obtain 1₅₂. Compound 1₅₄ was obtained as yellow (95% yield): ¹H NMR, 200 MHz, (CDCl₃) δ 1.39 (m, 4H), 1.57 (m, 4H), 2.41 (m, 4H), 2.62 (m, 8H), 3.22 (m, 8H), 6.89 (m, 6H), 7.27 (m, 6H). ESI-MS m/z 429 [M+Na⁺], 407 [M+H⁺] (100). Anal (C₂₆H₃₈N₄) C, H, N.

### 1,6-Bis(4-(pyridin-2-yl)piperazin-1-yl)hexane (Compound 1-55)

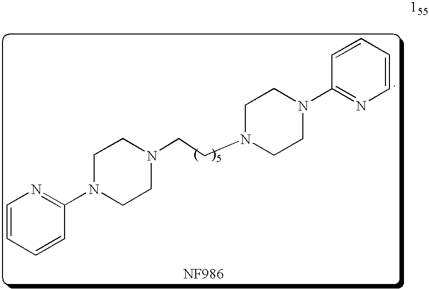

1 Starting from 1-(pyridin-2-yl)piperazine, the title compound was prepared following the procedure described to obtain 1₅₂. Compound 1₅₅ was obtained as yellow oil: ¹H NMR, 400 MHz, (CDCl₃) δ 1.32 (m, 4H), 1.50 (m, 4H), 2.32 (m, 4H), 2.49 (m, 8H), 3.50 (m, 8H), 6.56 (m, 4H), 7.40 (m, 2H), 8.14 (m, 2H). ESI-MS m/z 431 [M+Na⁺], 409 [M+H⁺] (100). Anal (C₂₄H₃₆N₆) C, H, N.

### 1,6-Bis(4-m-tolylpiperazin-1-yl)hexane (Compound 1-56)

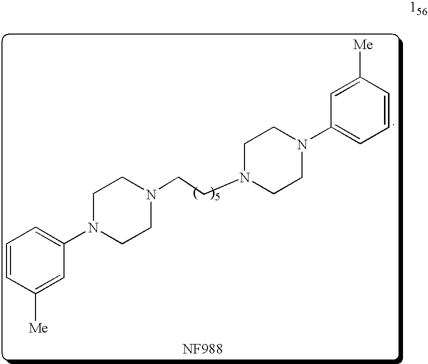

Starting from 4-(3-methylphenyl)piperazine, the title compound was prepared following the procedure described to obtain 1₅₂. Compound 1₅₆ was obtained as yellow oil (95% yield): ¹H NMR, 400 MHz, (CDCl₃) δ 1.37 (m, 4H), 1.55 (m, 4H), 2.31 (s, 6H), 2.39 (m, 4H), 2.60 (m, 4H), 3.20 (m, 4H), 6.71 (m, 6H), 7.14 (m, 2H). ESI-MS m/z 435 [M+H⁺] (100). Anal (C₂₈H₄₂N₄) C, H, N.

### Experimental Procedure for Compound 1-57

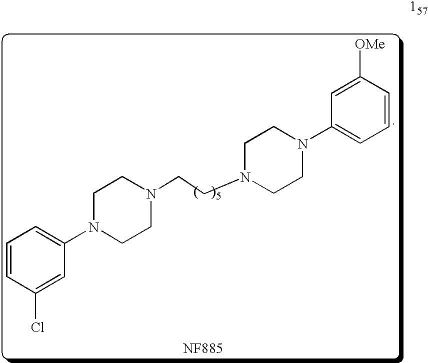

1-(6-Bromohexyl)-4-(3-methoxyphenyl)piperazine (42₁). To a solution of 3-methoxyphenylpiperazine (41₁) (200.0 mg, 1.04 mmol) in acetonitrile dry (15.0 mL), 1,6-dibromohexane (212.4 μL, 1.56 mmol) and TEA (145.0 μL, 1.04 mmol) was added and the mixture was stirred at room temperature overnight. The crude was extracted with dichloromethane (3×10 mL), dried and evaporated. The residue was chromatographed (15% n-hexane in EtOAc) to give 42₁, (65% yield): ¹H NMR, 300 MHz, (CDCl₃) δ 7.12 (m, 1H), 7.02 (m, 1H), 6.76 (m, 1H), 6.60 (m, 1H), 6.47 (m, 1H), 6.39 (m, 1H), 6.27 (m, 1H), 6.15 (m, 1H), 3.73 (m, 3H), 3.44 (m, 4H), 2.59 (m, 4H), 2.36 (m, 4H), 1.39 (m, 4H), 1.29 (m, 4H). ESI-MS m/z 493 [M+Na⁺], 471 [M+H⁺] (100). Anal (C₂₇H₃₉ClN₄O) C, H, N.

1-(3-Chlorophenyl)-4-(6-(4-(3-methoxyphenyl)piperazin-1-yl)hexyl)piperazine (Compound 1-57). To a solution of 42₁, (100.0 mg, 0.52 mmol) in acetonitrile dry (10.0 mL), 3-chlorophenylpiperazine (101.9 mg, 0.52 mmol) and TEA (72.5 μL, 0.52 mmol) was added and the mixture was stirred at room temperature overnight. The crude was extracted with dichloromethane (3×10 mL), dried and evaporated. The residue was chromatographed (10% methanol in chloroform) to give 1₅₇ (80% yield) as an yellow oil: ¹H NMR, 300 MHz, (CDCl₃) δ 1.35 (m, 4H), 1.54 (m, 4H), 2.39 (m, 4H), 2.59 (m, 8H), 3.20 (m, 8H), 3.78 (s, 3H), 6.42 (m, 2H), 6.53 (m, 1H), 6.78 (m, 2H), 6.86 (m, 1H), 7.15 (m, 2H). ESI-MS m/z 493 [M+Na⁺], 471 [M+H⁺] (100). Anal (C₂₇H₃₉ClN₄O) C, H, N.

### 1-Phenyl-4-(6-(4-(pyridin-2-yl)piperazin-1-yl)hexyl)piperazine (Compound 1-58)

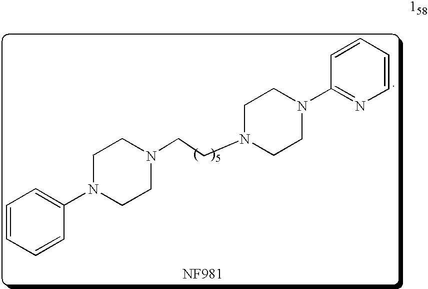

Starting from 42₂ and 1-(pyridin-2-yl)piperazine, the title compound was prepared following the procedure described to obtain 1₅₇. Compound 1₅₈ was obtained as yellow oil (60% yield): ¹H NMR, 400 MHz, (CDCl₃) δ 1.39 (m, 4H), 1.58 (m, 4H), 2.43 (m, 4H), 2.64 (m, 8H), 3.24 (m, 8H), 6.88 (m, 1H), 6.96 (m, 4H), 7.28 (m, 4H). ESI-MS m/z 430 [M+Na⁺], 408 [M+H⁺] (100). Anal (C₂₅H₃₇N₅) C, H, N.

### 1-(6-Methylpyridin-2-yl)-4-(6-(4-m-tolylpiperazin-1-yl)hexyl)piperazine (Compound 1-59)

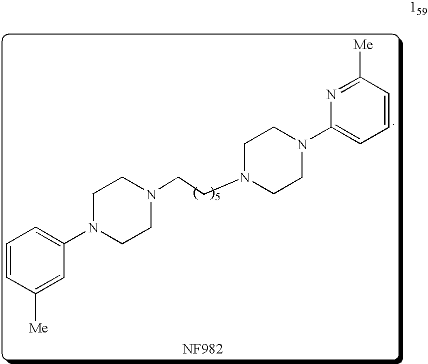

Starting from 42₃ and 1-(6-methylpyridin-2-yl)piperazine, the title compound was prepared following the procedure described to obtain 1₅₇. Compound 1₅₉ was obtained as yellow oil (63% yield): ¹H NMR, 400 MHz, (CDCl₃) δ 1.39 (m, 4H), 1.58 (m, 4H), 2.33 (s, 3H), 2.40 (m, 7H), 2.59 (m, 8H), 3.22 (m, 4H), 3.56 (m, 4H), 6.47 (m, 2H), 6.69 (m, 1H), 6.76 (m, 2H), 7.16 (m, 1H), 7.38 (m, 1H). ESI-MS m/z 458 [M+Na⁺], 436 [M+H⁺] (100). Anal (C₂₇H₄₁N₅) C, H, N.

### 1-(6-Methylpyridin-2-yl)-4-(6-(4-phenylpiperazin-1-yl)hexyl)piperazine (Compound 1-60)

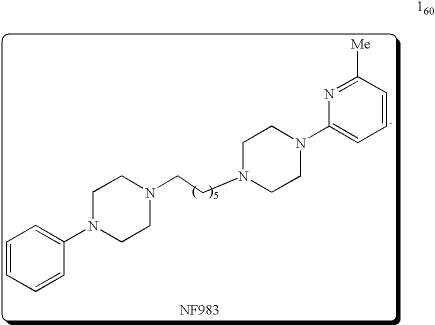

Starting from 42₂ and 1-(6-methylpyridin-2-yl)piperazine, the title compound was prepared following the procedure described to obtain 1₅₇. Compound 1₆₀ was obtained as yellow oil (60% yield): ¹H NMR, 400 MHz, (CDCl₃) δ1.39 (m, 4H), 1.58 (m, 4H), 2.41 (m, 7H), 3.23 (m, 4H), 3.58 (m, 4H), 6.47 (m, 2H), 6.87 (m, 1H), 6.95 (m, 2H), 7.28 (m, 2H), 7.38 (m, 1H). ESI-MS m/z 422 [M+H⁺] (100). Anal (C₂₆H₃₉N₅) C, H, N.

### 1-Phenyl-4-(6-(4-m-tolylpiperazin-1-yl)hexyl)piperazine (Compound 1-61)

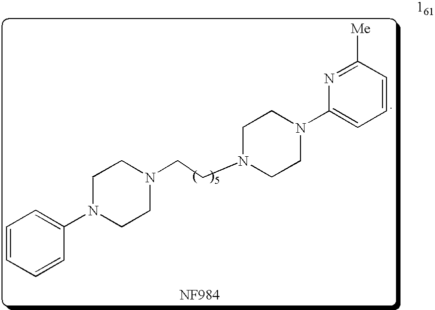

Starting from 42₂ and 4-(3-methylphenyl)piperazine, the title compound was prepared following the procedure described to obtain 1₅₇. Compound 1₆₁ was obtained as yellow oil (70% yield): ¹H NMR, 400 MHz, (CDCl₃) δ 1.39 (m, 4H), 1.57 (m, 4H), 2.34 (s, 3H), 2.42 (m, 4H), 2.63 (m, 8H), 3.23 (m, 8H), 6.73 (m, 1H), 6.78 (m, 2H), 6.88 (m, 1H), 6.96 (m, 2H), 7.18 (m, 1H), 7.29 (m, 2H). ESI-MS m/z 435 [M+Na⁺], 413 [M+H⁺] (100). Anal (C₂₇H₃₂N₄) C, H, N.

### 4-(4-(6-(4-Phenylpiperazin-1-yl)hexyl)piperazin-1-yl)quinoline (Compound 1-62)

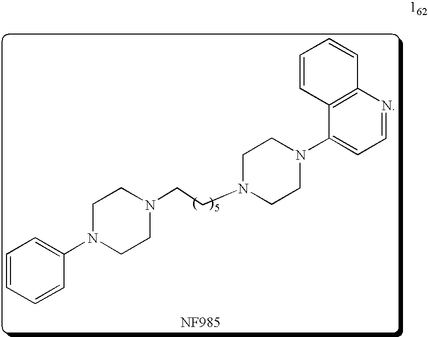

tert-Butyl-4-(quinolin-4-yl)piperazine-1-carboxylate (3₆). Starting from 4-bromoquinoline, the title compound was prepared following the procedure described to obtain 3₂. Compound 3₆ was obtained as yellow oil: ¹H NMR, 200 MHz, (CDCl₃) δ 1.38 (s, 9H), 2.98 (m, 4H), 3.56 (m, 4H), 6.62 (m, 1H), 7.54 (m, 1H), 7.72 (m, 1H), 7.85 (m, 1H), 7.93 (m, 1H), 8.57 (m, 1H). ESI-MS m/z 336 [M+Na⁺], 314 [M+H⁺] (100). Anal (C₁₈H₂₃N₃O₂) C, H, N.

4-(piperazin-1-yl)quinoline (4₅). Starting from 3₆, the title compound was prepared following the procedure described to obtain 4₁.

4-(4-(6-(4-Phenylpiperazin-1-yl)hexyl)piperazin-1-yl)quinoline (Compound 1-62). Starting from 42₂ and 4₅, the title compound was prepared following the procedure described to obtain 1₅₇. Compound 1₆₂ was obtained as yellow oil (65% yield): ¹H NMR, 400 MHz, (CDCl₃) δ 1.40 (m, 4H), 1.65 (m, 8H), 2.46 (m, 4H), 2.68 (m, 8H), 3.26 (m, 8H), 6.90 (m, 4H), 7.27 (m, 2H), 7.49 (m, 1H), 7.66 (m, 1H), 8.28 (m, 2H), 8.74 (m, 1H). ESI-MS m/z 480 [M+Na⁺], 458 [M+H⁺] (100). Anal (C₂₉H₃₉N₅) C, H, N.

### 4-(4-(6-(4-m-Tolylpiperazin-1-yl)hexyl)piperazin-1-yl)quinoline (Compound 1-63)

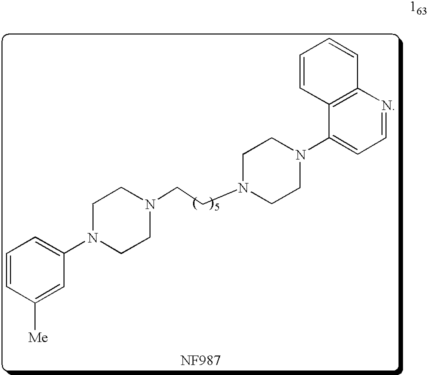

Starting from 42₃ and 4₅, the title compound was prepared following the procedure described to obtain 1₅₇. Compound 1₆₃ was obtained as yellow oil (70% yield): ¹H NMR, 400 MHz, (CDCl₃) δ 1.33 (m, 4H), 1.59 (m, 4H), 2.32 (s, 3H), 2.47 (m, 4H), 2.63 (m, 4H), 2.72 (m, 4H), 3.23 (m, 8H), 6.72 (m, 3H), 6.85 (m, 1H), 7.15 (m, 1H), 7.49 (m, 1H), 7.66 (m, 1H), 8.04 (m, 2H), 8.73 (m, 1H). ESI-MS m/z 494 [M+Na⁺], 472 [M+H⁺] (100). Anal (C₃₀H₄₁N₅) C, H, N.

### 1-(Pyridin-2-yl)-4-(6-(4-m-tolylpiperazin-1-yl)hexyl)piperazine (Compound 1-64)

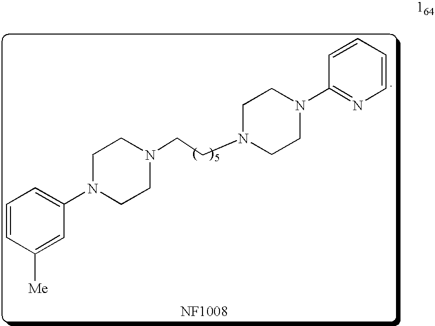

Starting from 42₃ and 1-(pyridin-2-yl)piperazine the title compound was prepared following the procedure described to obtain 1₅₇. Compound 1₆₄ was obtained as yellow (68% yield): ¹H NMR, 200 MHz, (CDCl₃) δ 1.36 (m, 4H), 1.55 (m, 4H), 2.30 (s, 3H), 2.38 (m, 4H), 2.57 (m, 8H), 3.19 (m, 4H), 3.55 (m, 4H), 6.66 (m, 5H), 7.14 (m, 1H), 7.43 (m, 1H), 8.18 (m, 1H). ESI-MS m/z 422 [M+H⁺] (100). Anal (C₂₆H₃₉N₅) C, H, N.

### 1-(3-Methoxyphenyl)-4-(6-(4-m-tolylpiperazin-1-yl)hexyl)piperazine (Compound 1-65)

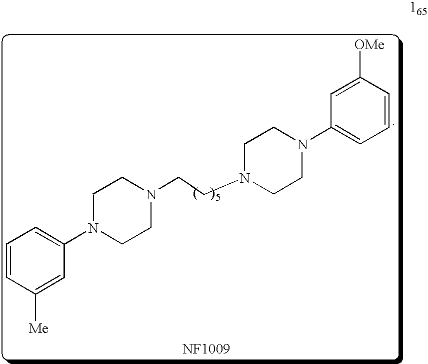

Starting from 42₃ and 4-(3-methoxyphenyl)piperazine, the title compound was prepared following the procedure described to obtain 1₅₇. Compound 1₆₅ was obtained as yellow (80% yield): ¹H NMR, 200 MHz, (CDCl₃) δ 1.37 (m, 4H), 1.55 (m, 4H), 2.31 (s, 3H), 2.39 (m, 4H), 2.60 (m, 8H), 3.20 (m, 8H), 3.78 (s, 3H), 6.43 (m, 2H), 6.53 (m, 2H), 6.71 (m, 2H), 7.15 (m, 2H), ESI-MS m/z 451 [M+H⁺) (100). Anal (C₂₈H₄₂N₄O) C, H, N.

### Biological Activity

### In Vitro Binding Studies

This example demonstrates the affinity of the compounds of the invention for the dopamine and serotonin receptor subtypes. These binding assays were carried out according to the procedure of Campiani et al.; J. Med. Chem. 2003 46 3822-3839.

Male CRL:CD(SD)BR-COBS rats (Charles River, Italy) were killed by decapitation; their brains were rapidly dissected into the various areas (striatum for D₁ and D₂ receptors, olfactory tubercle for D₃ receptors and cortex for 5-HT₂ receptors) and stored at −80° C. until assay. Tissues were homogenized in about 50 volumes of ice-cold Tris HCl, 50 mM, pH 7.4 (for D₁, D₂ and 5-HT₂ receptors), or 50 mM Hepes Na, pH 7.5 (for D₃ receptors) using an Ultra-Turrax TP-1810 homogenizer (2×20 s), and centrifuged at 48000 g for 10 minutes (Beckman Avanti J-25 centrifuge). Each pellet was resuspended in the same volume of fresh buffer, incubated at 37° C. for 10 minutes, and centrifuged again at 48000 g for 10 minutes. The pellet was then washed once by resuspension in fresh buffer and centrifuged as before. The resulting pellets were resuspended just before the binding assay in the appropriate incubation buffer (50 mM Tris HCl, pH 7.4, containing 10 μM pargyline, 0.1% ascorbic acid, 120 mM NaCl, 5 mM KCl, 2 mM CaCl₂, 1 mM MgCl₂ for D₁ and D₂ receptors; 50 mM Hepes Na, pH 7.5, containing 1 mM EDTA, 0.005% ascorbic acid, 0.1% albumin, 200 nM eliprodil for D₃ receptors and 50 mM Tris HCl, pH 7.7 for 5-HT₂ receptors).

[³H]-SCH 23390 (specific activity, 71.1 Ci/mmol; NEN), a reference substance for determination of binding to D₁ receptors, was assayed in a final incubation volume of 0.5 mL, consisting of 0.25 mL of membrane suspension (2 mg of tissue/sample), 0.25 mL of [³H]ligand (0.4 nM), and 10 μL of displacing agent or solvent. Non-specific binding was obtained in the presence of 10 μM (−)cis-flupentixol.

[³H]-Spiperone (specific activity, 16.5 Ci/mmol; NEN), a reference substance for determination of binding to D₂ receptors, was assayed in a final incubation volume of 1 mL, consisting of 0.5 mL of membrane suspension (1 mg of tissue/sample), 0.5 mL of [³H]-ligand (0.2 nM), and 20 μL of displacing agent or solvent. Non-specific binding was obtained in the presence of 100 μM (−)sulpiride.

[³H]-7-OH-DPAT (specific activity, 159 Ci/mmol; Amersham), a reference substance for determination of binding to D₃ receptors, was assayed in a final incubation volume of 1 mL, consisting of 0.5 mL of membrane suspension (10 g of prot./sample of rat cloned dopamine receptor D3 in Sf9 cells (Signal Screen)), 0.5 mL of [³H]-ligand (0.7 nM), and 20 μL of displacing agent or solvent. Non-specific binding was obtained in the presence of 1 μM dopamine.

[³H]-Ketanserin (specific activity, 63.3 Ci/mmol; Amersham), a reference substance for determination of binding to 5-HT₂ receptors, was assayed in a final incubation volume of 1 mL, consisting of 0.5 mL of membrane suspension (5 mg of tissue/sample), 0.5 mL of [³H]-ligand (0.7 nM), and 20 μL of displacing agent or solvent. Non-specific binding was obtained in the presence of 1 μM methysergide.

Incubations (15 minutes at 37° C. for D₁, D₂ and 5-HT₂ receptors; 60 minutes at 25° C. for D₃ receptors) were stopped by rapid filtration under vacuum through GF/B (for D₁, D₂ and 5-HT₂ receptors) or GF/C (for D₃ receptors) filters, which were then washed with 12 mL (4×3 times) of ice-cold buffer (50 mM Tris HCl, pH 7.7) using a Brandel M-48R cell harvester. The radioactivity trapped on the filters was counted in 4 mL of Ultima Gold MV (Packard) in a LKB 1214 rack beta liquid scintillation spectrometer with a counting efficiency of 50%.

1-45. (canceled)

46. An aryl piperazine derivative represented by Formula I 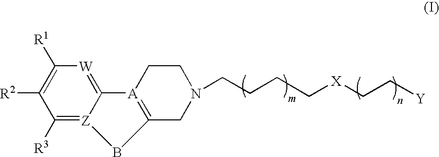
an enantiomer thereof or a mixture of its enantiomers, or a pharmaceutically acceptable salt thereof, or an N-oxide thereof, wherein,
R¹, R² and R³, independently of one another, represent hydrogen, alkyl, cycloalkyl, cycloalkyl-alkyl, alkenyl, hydroxy, alkoxy, cycloalkoxy, halo, haloalkyl, haloalkoxy, amino, nitro cyano and/or carboxy; [symbol]
represents an optional double bond;
if [symbol] represents a single bond, then
A represents CH or N;
if [symbol] represents a double bond, then
A represents C;
—B— may be absent or present:
—B— is absent; and
Z represents CH or N; or
—B— is present and represents a methylene bridge (—CH₂—), an ethylene bridge (—CH═CH—), or a bridge —NH—, attached as indicated in the figure; and
Z represents C (carbon);
W represents CH, N or CR⁴, wherein R⁴ represents represent hydrogen, alkyl, cycloalkyl, cycloalkyl-alkyl, alkenyl, hydroxy, alkoxy, cycloalkoxy, halo, haloalkyl, haloalkoxy, amino, nitro or cyano;
m and n, independently of each other, is 0, 1 or 2; and
X may be absent or present:
X is present and represents O, S, NR′, CO, SO₂, CH₂, CH₂—O, O—CH₂, CH₂—S, S—CH₂, CH₂—NR′, CH₂—CO, CH₂—SO₂, NR′—CO, CO—NR′, NR′—SO₂, SO₂—NR′, CH₂—CH₂, O—CO, CO—O, O—CH═CH, S—CH═CH, NR′—CH═CH, CO—CH═CH, SO₂—CH═CH, CH₂—O—CH═CH, CH₂—S—CH═CH, CH₂—NR′—CH═CH, CH₂—CO—CH═CH, CONHCH₂CH₂ or CH₂—SO₂—CH═CH, wherein R′ represents hydrogen or alkyl; and
Y represents phenyl or an aromatic monocyclic or polycyclic heterocyclic group, which phenyl or heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano; or
Y represents a hydrogenated heterocyclic group, which hydrogenated heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano; or
Y represents a group of formula III 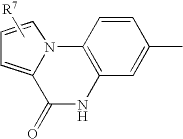
wherein
R⁷ represents hydrogen, alkyl, alkoxy, halo or haloalkyl; or
X is absent; and
Y represents a diazacyclic group of Formula II, 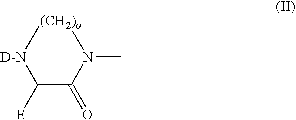
wherein,
o is 1, 2 or 3;
D represents alkyl, cycloalkyl, cycloalkyl-alkyl, hydroxy, alkoxy, cycloalkoxy, halo, haloalkyl, haloalkoxy, amino, nitro and cyano; and
E represents alkyl, cycloalkyl, cycloalkyl-alkyl, hydroxy, alkoxy, cycloalkoxy, halo, haloalkyl, haloalkoxy, amino, nitro and cyano; or
D and E together with the diazacyclic group form a fused ring system, which fused ring system may optionally be substituted one or more times with substituents selected from the group consisting of alkyl, cycloalkyl, cycloalkyl-alkyl, hydroxy, alkoxy, cycloalkoxy, halo, haloalkyl, haloalkoxy, amino, nitro and cyano;
or Y represents a group of formula IV 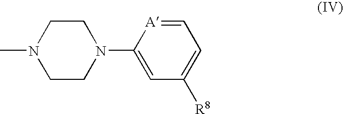
wherein A′ represents CH or N; and
R⁸ represents hydrogen, alkyl, alkoxy, halo or haloalkyl.

47. The aryl piperazine derivative of claim 46, wherein [symbol]
represents a single bond, and
A represents CH or N.

48. The aryl piperazine derivative of claim 46, wherein [symbol]
represents a double bond, and
A represents C (carbon).

49. The aryl piperazine derivative of claim 46, wherein
W represents CH, N or CR⁴, wherein R⁴ represents represent hydrogen, alkyl, cycloalkyl, cycloalkyl-alkyl, alkenyl, hydroxy, alkoxy, cycloalkoxy, halo, haloalkyl, haloalkoxy, amino, nitro or cyano.

50. The aryl piperazine derivative of claim 46, wherein
—B— is absent, and
Z represents CH or N.

51. The aryl piperazine derivative of claim 46, wherein
—B— is present and represents a methylene bridge (—CH₂—), an ethylene bridge (—CH═CH—), or a bridge —NH—, attached as indicated in the figure; and
Z represents C (carbon).

52. The aryl piperazine derivative of claim 51, wherein
—B— is present and represents a methylene bridge (—CH₂—), an ethylene bridge (—CH═CH—), or a bridge —NH—, attached as indicated in the figure; and
Z represents C (carbon); and
W represents CR⁴, wherein R⁴ represents represent hydrogen, alkyl, alkoxy, halo, haloalkyl, haloalkoxy, amino, nitro or cyano.

53. The aryl piperazine derivative of claim 46, wherein
m and n, independently of each other, is 0, 1 or 2.

54. The aryl piperazine derivative of claim 53, wherein
m is 1 or 2; and
n is 0 or 2.

55. The aryl piperazine derivative of claim 46, wherein
R¹, R² and R³, independently of one another, represent hydrogen, alkyl, cycloalkyl, cycloalkyl-alkyl, alkenyl, hydroxy, alkoxy, cycloalkoxy, halo, haloalkyl, haloalkoxy, amino, nitro cyano and/or carboxy;

56. The aryl piperazine derivative of claim 55, wherein
R¹ represents alkyl, cycloalkyl, cycloalkyl-alkyl, hydroxy, alkoxy, cycloalkoxy, halo, haloalkyl, haloalkoxy, amino, nitro, cyano or carboxy; and
R² and R³ represent hydrogen.

57. The aryl piperazine derivative of claim 55, wherein
R² represents alkyl, cycloalkyl, cycloalkyl-alkyl, hydroxy, alkoxy, cycloalkoxy, halo, haloalkyl, haloalkoxy, amino, nitro or cyano; and
R¹ and R³ represent hydrogen.

58. The aryl piperazine derivative of claim 46, wherein
X is present and represents O, S, NR′, CO, SO₂, CH₂, CH₂—O, O—CH₂, CH₂—S, S—CH₂, CH₂—NR′, CH₂—CO, CH₂—SO₂, NR′—CO, CO—NR′, CH₂—CH₂, O—CO, CO—O, O—CH═CH, S—CH═CH, NR′—CH═CH, CO—CH═CH, SO₂—CH═CH, CH₂—O—CH═CH, CH₂—S—CH═CH, CH₂—NR′—CH═CH, CH₂—CO—CH═CH, CONHCH₂CH₂ or CH₂—SO₂—CH═CH, wherein R′ represents hydrogen or alkyl.

59. The aryl piperazine derivative of claim 58, wherein X represents O, CH₂—O, O—CH₂, CH₂—S, S—CH₂, CH₂—NR′, CH₂—CO, CH₂—SO₂, NR′—CO, CO—NR′, NR′—SO₂, SO₂—NR′, O—CO, or CH₂—O—CH═CH; wherein R′ represents hydrogen or alkyl.

60. The aryl piperazine derivative of claim 59, wherein X represents O, CH₂—O, NR′—CO, CO—NR′, NR′—SO₂ or O—CO; wherein R′ represents hydrogen or alkyl.

61. The aryl piperazine derivative of claim 58, wherein
Y represents phenyl or an aromatic monocyclic or polycyclic heterocyclic group, which phenyl or heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano; or
Y represents a hydrogenated heterocyclic group, which hydrogenated heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano.

62. The aryl piperazine derivative of claim 61, wherein Y represents phenyl, which phenyl group may optionally be substituted one or more times with substituents selected from the group consisting of alkyl, cycloalkyl, cycloalkyl-alkyl, hydroxy, alkoxy, cycloalkoxy, halo, haloalkyl, haloalkoxy, amino, nitro and cyano.

63. The aryl piperazine derivative of claim 58, wherein Y represents an aromatic monocyclic heterocyclic group selected from furanyl, thienyl, pyrrolyl, oxazolyl, imidazolyl, pyridyl, pyridazinyl and pyrimidinyl, which aromatic monocyclic heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano.

64. The aryl piperazine derivative of claim 63, wherein Y represents furanyl, thienyl or pyridyl, which aromatic monocyclic heterocyclic group may optionally be substituted one or two times with substituents selected from the group consisting of alkyl, alkoxy, chloro, trifluoromethyl and trifluoromethoxy.

65. The aryl piperazine derivative of claim 58, wherein Y represents an aromatic bicyclic heterocyclic group selected from indolyl, isoindolyl, benzo[b]furanyl, benzo[b]thienyl, benzimidazolyl, benzthiazolyl, quinolinyl and isoquinolinyl, which aromatic bicyclic heterocyclic group may optionally be substituted one or two times with substituents selected from the group consisting of alkyl, cycloalkyl, cycloalkyl-alkyl, hydroxy, alkoxy, cycloalkoxy, halo, haloalkyl, haloalkoxy, amino, nitro and cyano.

66. The aryl piperazine derivative of claim 65, wherein Y represents indolyl, in particular indol-2-yl or indol-3-yl; benzo[b]furanyl, in particular benzo[b]furan-2-yl or benzo[b]furan-3-yl; benzo[b]thienyl, in particular benzo[b]thien-2-yl or benzo[b]thien-3-yl; quinolinyl in particular quinolin-2-yl, quinolin-3-yl or quinolin-4-yl; or isoquinolinyl, in particular isoquinolin-1-yl, isoquinolin-3-yl, or isoquinolin-4-yl; which aromatic bicyclic heterocyclic group may optionally be substituted one or two times with substituents selected from alkyl, cycloalkyl, cycloalkyl-alkyl, hydroxy, alkoxy, cycloalkoxy, halo, haloalkyl, haloalkoxy, amino, nitro and cyano.

67. The aryl piperazine derivative of claim 66, wherein Y represents indolyl, in particular indol-2-yl or indol-3-yl; benzo[b]furanyl, in particular benzo[b]furan-2-yl or benzo[b]furan-3-yl; quinolinyl, in particular quinolin-2-yl, quinolin-3-yl or quinolin-4-yl; or isoquinolinyl, in particular isoquinolin-1-yl, isoquinolin-3-yl, or isoquinolin-4-yl; which benzo[b]furanyl or isoquinolinyl may optionally be substituted one or two times with substituents selected from alkyl, hydroxy, alkoxy, chloro, trifluoromethyl, trifluoromethoxy, amino, nitro and cyano.

68. The aryl piperazine derivative of claim 67, wherein Y represents indol-2-yl, benzo[b]furan-2-yl or isoquinolin-3-yl; which benzo[b]furanyl or isoquinolinyl may optionally be substituted one or two times with substituents selected from alkyl, hydroxy, alkoxy, chloro, trifluoromethyl, trifluoromethoxy, amino, nitro and cyano.

69. The aryl piperazine derivative of claim 68, wherein Y represents indolyl, benzo[b]furanyl, or isoquinolinyl.

70. The aryl piperazine derivative of claim 58, wherein Y represents a hydrogenated heterocyclic group, which hydrogenated heterocyclic group may optionally be substituted one or more times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano.

71. The aryl piperazine derivative of claim 70, wherein Y represents tetrahydroquinolinyl or tetrahydroisoquinolinyl, which heterocyclic group may optionally be substituted one or two times with substituents selected from the group consisting of alkyl, cycloalkyl, cycloalkyl-alkyl, alkenyl, hydroxy, alkoxy, cycloalkoxy, halo, haloalkyl, haloalkoxy, amino, nitro and cyano.

72. The aryl piperazine derivative of claim 71, wherein Y represents tetrahydroquinolinyl or tetrahydroisoquinolinyl.

73. The aryl piperazine derivative of claim 58, wherein
X represents O, CH₂—O, NH—CO, CO—NH, NR′—SO₂ or CO—O; and
Y represents phenyl, methyl-phenyl, pyridyl, indolyl, methyl-indolyl, benzo[b]furanyl, tetrahydroquinolinyl, isoquinolinyl, or tetrahydroisoquinolinyl.

74. The aryl piperazine derivative of claim 73, wherein
X represents O, CH₂—O, NH—CO, CO—NH, NR′—SO₂ or CO—O;
Y represents phenyl, methyl-phenyl, pyridyl, methyl-pyridyl, indolyl, methyl-indolyl, benzo[b]furanyl, tetrahydroquinolinyl, isoquinolinyl, or tetrahydroisoquinolinyl;
R¹ represents alkyl, cycloalkyl, cycloalkyl-alkyl, hydroxy, alkoxy, cycloalkoxy, halo, haloalkyl, haloalkoxy, amino, nitro or cyano; and
R² and R³ represent hydrogen.

75. The aryl piperazine derivative of claim 73, which is N-[4-[4-(3-Trifluoromethylphenyl)piperazin-1-yl]butyl]indole-2-carboxamide;
N-[2-(1H-Indol-3-yl)ethyl]-3-(4-m-tolylpiperazin-1-yl)propanamide;
N-[2-(1H-Indol-3-yl)ethyl]-3-[4-(3-methoxyphenyl)piperazin-1-yl]propanamide;
Benzo[b]furan-2-carboxylic acid {4-[4-(3-methoxy-phenyl)-piperazin-1-yl]-butyl}-amide;
N-[4-[4-(3-Cyanophenyl)piperazin-1-yl]butyl]benzo[b]furan-2-carboxamide;
Benzo[b]furan-2-carboxylic acid {4-[4-(3-chloro-phenyl)-piperazin-1-yl]-butyl}-amide;
Benzo[b]furan-2-carboxylic acid {4-[4-(3-carboxy-phenyl)-piperazin-1-yl]-butyl}-amide;
N-[4-[4-(m-Tolyl)piperazin-1-yl]butyl]benzo[b]furan-2-carboxamide;
Isoquinoline-3-carboxylic acid {4-[4-(3-cyano-phenyl)-piperazin-1-yl]-butyl}-amide;
N-[4-[4-(3-Chlorophenyl)piperazin-1-yl]butyl]isoquinoline-3-carboxamide;
N-[4-[4-(m-Tolyl)piperazin-1-yl]butyl]isoquinoline-3-carboxamide;
N-[4-[4-(3-Methoxyphenyl)piperazin-1-yl]butyl]isoquinoline-3-carboxamide;
3-[5-[4-(3-Chlorophenyl)piperazin-1-yl]pentyloxy]isoquinoline;
3-{5-[4-(3-Methoxy-phenyl)-piperazin-1-yl]-pentyloxy}-isoquinoline;
3-[5-(4-m-Tolylpiperazin-1-yl)pentyloxy]isoquinoline;
3-{5-[4-(3-Cyano-phenyl)-piperazin-1-yl]-pentyloxy}-isoquinoline;
N-[4-(1,2,3,4-Tetrahydro-5-methoxy-β-carbolin-2-yl)butyl]isoquinoline-3-carboxamide;
N-[4-(3,4-Dihydro-6-methoxypyrazino[1,2-a]indol-2(1H)-yl)butyl]isoquinoline-3-carboxamide;
N-[4-[4-(Pyridin-2-yl)piperazin-1-yl]butyl]isoquinoline-3-carboxamide;
1,2,3,4-Tetrahydro-quinoline-2-carboxylic acid[4-(4-phenyl-piperazin-1-yl)-butyl]-amide;
(S)-(−)-N-[4-[4-(m-Tolyl)piperazin-1-yl]butyl]-1,2,3,4-tetrahydroisoquinoline-2-carboxamide;
(R)-(+)-N-[4-[4-(m-Tolyl)piperazin-1-yl]butyl]-1,2,3,4-tetrahydroisoquinoline-2-carboxamide;
1H-Indole-2-carboxylic acid {4-[4-(2,4-dichloro-phenyl)-piperazin-1-yl]-butyl}-amide;
5-Chloro-1H-indole-2-carboxylic acid {4-[4-(2,4-dichloro-phenyl)-piperazin-1-yl]-butyl}-amide;
Isoquinoline-3-carboxylic acid {4-[4-(2,3-dichloro-phenyl)-piperazin-1-yl]-butyl}-amide;
3-{4-[4-(2,3-Dichloro-phenyl)-piperazin-1-yl]-butoxy}-isoquinoline;
3-{5-[4-(2,3-Dichloro-phenyl)-piperazin-1-yl]-pentyloxy}-isoquinoline;
4-[4-(2,3-Dichlorophenyl)piperazin-1-yl]butyl 1H-indole-2-carboxylate;
N-(4-(4-(Phenylpiperazin-1-yl)butyl)benzo[b]furan-2-carboxamide;
Benzo[b]furan-2-carboxylic acid {4-[4-(2,3-dimethyl-phenyl)-piperazin-1-yl]-butyl}-amide;
N-(4-(4-(3-Methoxyphenyl)piperazin-1-yl)butyl)benzo[b]furan-2-carboxamide;
N-(4-(4-(6-Methylpyridin-2-yl)piperazin-1-yl)butyl)isoquinoline-3-carboxamide;
N-(4-(4-Phenylpiperazin-1-yl)butyl)isoquinoline-3-carboxamide;
N-(4-(4-(6-Methylpyridin-2-yl)piperazin-1-yl)butyl)benzo[b]furan-2-carboxamide;
N-(4-(4-Phenylpiperazin-1-yl)butyl)quinoline-2-carboxamide;
N-(4-(4-m-Tolylpiperazin-1-yl)butyl)quinoline-2-carboxamide;
N-(4-(4-(3-Methoxyphenyl)piperazin-1-yl)butyl)-1-methyl-1H-indole-2-carboxamide;
N-(4-(4-(3-Methoxyphenyl)piperazin-1-yl)butyl)-1H-indole-3-carboxamide;
(S)-1,2,3,4-Tetrahydro-N-(4-(4-phenylpiperazin-1-yl)butyl)quinoline-2-carboxamide;
N-(4-(4-m-Tolylpiperazin-1-yl)butyl)picolinamide;
N-(4-(4-(Quinolin-3-yl)piperazin-1-yl)butyl)isoquinoline-3-carboxamide;
N-(4-(4-(3-Methoxyphenyl)piperazin-1-yl)butyl)-6-methylpyridine-2-carboxamide;
N-(4-(4-(3-Methoxyphenyl)piperazin-1-yl)butyl)quinoline-3-carboxamide;
N-(4-(4-(Pyridin-2-yl)piperazin-1-yl)butyl)quinoline-3-carboxamide;
N-(4-(4-Phenylpiperazin-1-yl)butyl)picolinamide;
N-(4-(4-(3-Methoxyphenyl)piperazin-1-yl)butyl)picolinamide;
N-(4-(4-(3-Methoxyphenyl)piperazin-1-yl)butyl)benzamide;
N-(4-(4-m-Tolylpiperazin-1-yl)butyl)benzamide;
N-(4-(4-Phenylpiperazin-1-yl)butyl)nicotinamide;
N-(4-(4-(6-Methylpyridin-2-yl)piperazin-1-yl)butyl)benzamide;
N-(4-(4-(6-Methoxypyridin-2-yl)piperazin-1-yl)butyl)benzamide;
N-(4-(4-(6-Methoxypyridin-2-yl)piperazin-1-yl)butyl)picolinamide; or
N-(4-(4-(6-Methylpyridin-2-yl)piperazin-1-yl)butyl)picolinamide;
or a pharmaceutically acceptable salt thereof.

76. The aryl piperazine derivative of claim 58, wherein
Y represents a group of formula III 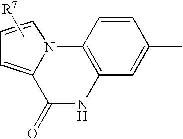
wherein
R⁷ represents hydrogen, alkyl, alkoxy, halo or haloalkyl.

77. The aryl piperazine derivative of claim 76, which is
7-[4-[4-(2,3-Dichloro-phenyl)-piperazin-1-yl]-butoxy]-pyrrolo[1,2-a]quinoxalin-4(5H)-one;
7-(5-(4-Phenylpiperazin-1-yl)pentyloxy)pyrrolo[1,2-a]quinoxalin-4(5H)-one; or
7-(4-(4-Phenylpiperazin-1-yl)butoxy)pyrrolo[1,2-a]quinoxalin-4(5H)-one;
or a pharmaceutically acceptable salt thereof.

78. The aryl piperazine derivative of claim 46, wherein
X is absent; and
Y represents a diazacyclic group of Formula II, 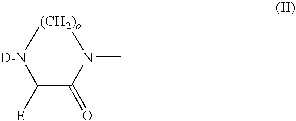
wherein,
o is 1, 2 or 3;
D represents alkyl, cycloalkyl, cycloalkyl-alkyl, hydroxy, alkoxy, cycloalkoxy, halo, haloalkyl, haloalkoxy, amino, nitro and cyano; and
E represents alkyl, cycloalkyl, cycloalkyl-alkyl, hydroxy, alkoxy, cycloalkoxy, halo, haloalkyl, haloalkoxy, amino, nitro and cyano; or
D and E together with the diazacyclic group form a fused ring system, which fused ring system may optionally be substituted one or more times with substituents selected from the group consisting of alkyl, cycloalkyl, cycloalkyl-alkyl, hydroxy, alkoxy, cycloalkoxy, halo, haloalkyl, haloalkoxy, amino, nitro and cyano.

79. The aryl piperazine derivative of claim 78, wherein
Y represents a bicyclic heterocyclic group (i.e. fused ring system) selected from the following group: 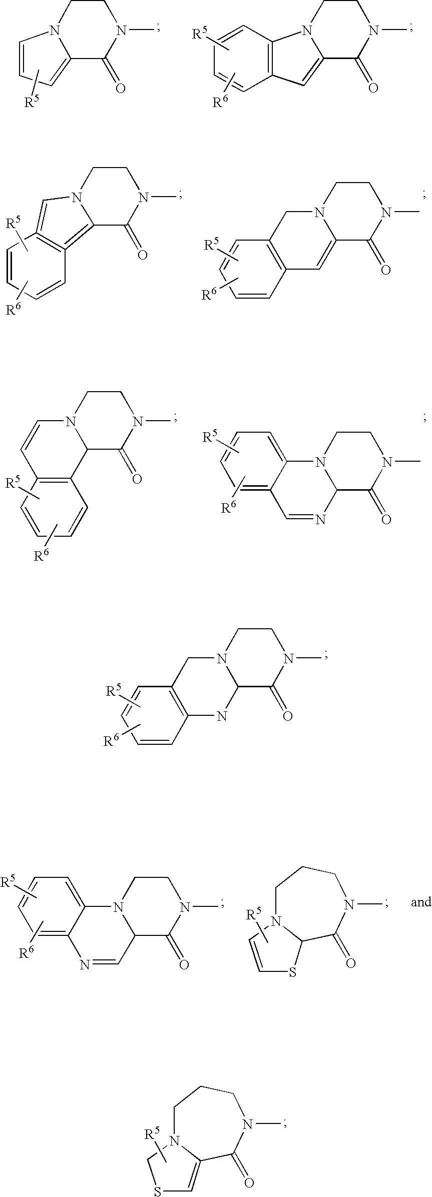
wherein R⁵ and R⁶, independently of each other, represent hydrogen, alkyl, cycloalkyl, cycloalkyl-alkyl, hydroxy, alkoxy, cycloalkoxy, halo, haloalkyl, haloalkoxy, amino, nitro and/or cyano.

80. The aryl piperazine derivative of claim 79, wherein
Y represents a bicyclic heterocyclic group selected from 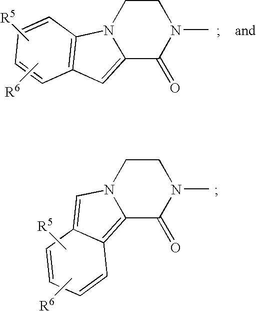
wherein R⁵ and R⁶, independently of each other, represent hydrogen, alkyl, cycloalkyl, cycloalkyl-alkyl, hydroxy, alkoxy, cycloalkoxy, halo, haloalkyl, haloalkoxy, amino, nitro and/or cyano.

81. The aryl piperazine derivative of claim 80, wherein
Y represents 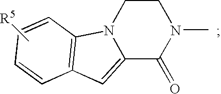
wherein
R⁵ represents hydrogen, alkyl, halo, trifluoromethyl or trifluoromethoxy.

82. The aryl piperazine derivative of claim 81, which is
2-{4-[4-(3-Cyano-phenyl)-piperazin-1-yl]-butyl}-3,4-dihydro-2H-pyrazino[1,2-a]indol-1-one;
2-[4-[4-(3-Chlorophenyl)piperazin-1-yl]butyl]-3,4-dihydropyrazino[1,2-a]indol-1(2H)-one;
2-{4-[4-(3-Methoxy-phenyl)-piperazin-1-yl]-butyl}-3,4-dihydro-2H-pyrazino[1,2-a]indol-1-one;
2-[4-(4-m-Tolyl)piperazin-1-yl]butyl]-3,4-dihydropyrazino[1,2-a]indol-1(2H)-one;
3,4-Dihydro-2-[4-(3,4-dihydro-6-methoxypyrazino[1,2-a]indol-2(1H)-yl)butyl]pyrazino[1,2-a]indol-1(2H)-one;
2-{4-[4-(2-Methoxy-phenyl)-piperazin-1-yl]-butyl}-3,4-dihydro-2H-pyrazino[1,2-a]indol-1-one; or
2-{4-[4-(2,3-Dichloro-phenyl)-piperazin-1-yl]-butyl}-3,4-dihydro-2H-pyrazino[1,2-a]indol-1-one;
or a pharmaceutically acceptable salt thereof.

83. The aryl piperazine derivative of claim 46, wherein
X is absent; and
Y represents a group of formula IV 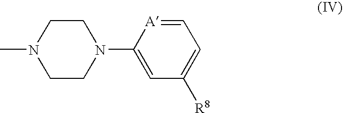
wherein A′ represents CH or N; and
R⁸ represents hydrogen, alkyl, alkoxy, halo or haloalkyl.

84. The aryl piperazine derivative of claim 83, which is
1,6-Bis(4-(3-chlorophenyl)piperazin-1-yl)hexane;
1,6-Bis(4-(3-methoxyphenyl)piperazin-1-yl)hexane;
1,6-Bis(4-phenylpiperazin-1-yl)hexane;
1-(3-Chlorophenyl)-4-(6-(4-(3-methoxyphenyl)piperazin-1-yl)hexyl)piperazine;
1-Phenyl-4-(6-(4-(pyridin-2-yl)piperazin-1-yl)hexyl)piperazine;
1-(6-Methylpyridin-2-yl)-4-(6-(4-m-tolylpiperazin-1-yl)hexyl)piperazine;
1-(6-Methylpyridin-2-yl)-4-(6-(4-phenylpiperazin-1-yl)hexyl)piperazine;
1-Phenyl-4-(6-(4-m-tolylpiperazin-1-yl)hexyl)piperazine;
4-(4-(6-(4-Phenylpiperazin-1-yl)hexyl)piperazin-1-yl)quinoline;
1,6-Bis(4-(pyridin-2-yl)piperazin-1-yl)hexane;
4-(4-(6-(4-m-Tolylpiperazin-1-yl)hexyl)piperazin-1-yl)quinoline;
1,6-Bis(4-m-tolylpiperazin-1-yl)hexane;
1-(Pyridin-2-yl)-4-(6-(4-m-tolylpiperazin-1-yl)hexyl)piperazine; or
1-(3-Methoxyphenyl)-4-(6-(4-m-tolylpiperazin-1-yl)hexyl)piperazine;
or a pharmaceutically acceptable salt thereof.

85. A pharmaceutical composition comprising a therapeutically effective amount of an aryl piperazine derivative of claim 46, or a pharmaceutically-acceptable addition salt thereof, or a prodrug thereof, together with at least one pharmaceutically acceptable carrier or diluent.

86. A method of diagnosis, treatment, prevention or alleviation of a disease or a disorder or a condition of a living animal body, including a human, which disorder, disease or condition is responsive to modulation of the dopamine and serotonin receptors, in particular the D₃, D₂-like and 5-HT₂ receptor subtypes, preferably the dopamine D₃ receptor subtype and/or the D₃/5-HT_{1A} or D₃/5-HT_{2A} receptor subtypes, which method comprises the step of administering to such a living animal body in need thereof, a therapeutically effective amount of an aryl piperazine derivative according to claim 1, a pharmaceutically acceptable salt thereof, or a prodrug thereof.

87. The method according to claim 86, wherein the disease or a disorder or a condition is a neurological or psychiatric disorders, in particular psychotic disorders, schizophrenia, depression, Parkinson's disease, Huntington's disease, movement disorders, dystonia, anxiety, restlessness, obsessive-compulsive disorders, mania, geriatric disorders, dementia, sexual dysfunction, musculo-skeletal pain symptoms, pain associated with fibromyalgia, sleep disorders, substance abuse or addiction and withdrawal symptoms in drug addicts, cocaine abuse or addiction.

88. The method according to claim 87, wherein the disease or a disorder or a condition is a neurological or psychiatric disorder, in particular a psychotic disorder, preferably schizophrenia.
